(19) United States
(12) Patent Application Publication
(10) Pub. No.: US 2025/0206763 A1
(43) Pub. Date: Jun. 26, 2025

(54) ENPP1 INHIBITOR

(71) Applicant: SHANGHAI QILU PHARMACEUTICAL RESEARCH AND DEVELOPMENT CENTRE LTD., Pudong New Area, Shanghai (CN)

(72) Inventors: Qi YAN, Shanghai (CN); Jianfeng PAN, Shanghai (CN); Daqing SUN, Shanghai (CN)

(73) Assignee: SHANGHAI QILU PHARMACEUTICAL RESEARCH AND DEVELOPMENT CENTRE LTD., Pudong New Area, Shanghai (CN)

(21) Appl. No.: 18/848,394

(22) PCT Filed: Apr. 10, 2023
(86) PCT No.: PCT/CN2023/087272

§ 371 Date: Sep. 18, 2024

(30) Foreign Application Priority Data
Apr. 11, 2022 (CN) 202210373013.8
Jul. 26, 2022 (CN) 202210883937.2
Sep. 26, 2022 (CN) 202211173156.0

(51) Int. Cl. C07F 5/02; A61K 31/4745; A61K 31/675; A61K 31/69; A61P 35/00; C07D 491/147; C07F 9/6561

(52) U.S. Cl. CPC C07F 5/025; A61K 31/4745; A61K 31/675; A61K 31/69; A61P 35/00; C07D 491/147; C07F 9/6561

(57) ABSTRACT

Disclosed are a novel compound as represented by formula (I-b) having an ENPP1 inhibitory activity, a pharmaceutical composition comprising the compound, an intermediate useful for preparing the compound, and a method for treating cell proliferative diseases, such as tumors, by using the above-mentioned compound.

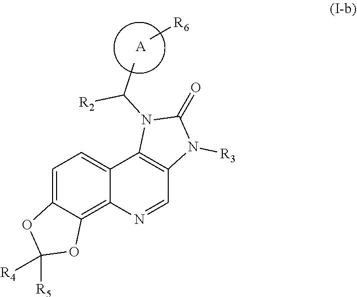

### TECHNICAL FIELD

The present application belongs to the field of medicinal chemistry and specifically relates to a novel compound having an ENPP1 inhibitory activity, a pharmaceutical composition comprising the compound, an useful intermediate for preparing the compound, and a method for treating cell proliferative diseases, such as solid tumors, by using the compound of the present invention.

### BACKGROUND

Tumor is one of major diseases that seriously endanger human life and health, and has the characteristics of hyperproliferation and abnormal differentiation of cells. Lung cancer, colon/rectal cancer, gastric cancer, liver cancer and the like have the highest incidence and mortality rates among all types of malignant tumors. As the incidence and mortality rates of malignant tumors increase every year, there is a growing demand for malignant tumor therapy.

Microbial and viral DNA in infected mammalian cells can induce endogenous potent immune responses by stimulating interferon secretion. Endoplasmic reticulum (ER) receptor protein (STING) is a necessary factor for the immune response to cytoplasmic DNA. Recent studies have shown that cyclic GMP-AMP dinucleotide synthetase (cGAS) endogenously catalyzes the synthesis of cGAMP under activation conditions after binding to DNA. cGAMP is a cytoplasmic DNA sensor, which acts as a second messenger to stimulate INF-β sensing via STING, mediates the activation of TBK1 and IRF-3, and then initiates INF-β gene transcription. Recently, it was reported that recombinant cGAS catalyzes the synthesis of cyclic cGMP-AMP dinucleotide, i.e., cGAMP, after binding to DNA. The crystal structure of cGAS-DNA complex has also been reported. cGAMP has an important role in antiviral immunity, and cGAMP binds to STING to lead to an activation of transcription factor IRF3 and a production of β-interferon.

Cyclic guanosine monophosphate-adenosine monophosphate (cGAMP) activates the pathway of stimulating factor of interferon genes (STING), which an important innate anticancer immune pathway. Cyclic dinucleotide synthetase (cGAS) is an important cytoplasmic DNA receptor in innate immune pathway. cGAMP acts as a second messenger molecule to induce the production of interferon IFN-β and other cytokines through the STING protein pathway on the endoplasmic reticulum membrane, thereby regulating downstream protein expression, inducing growth arrest and apoptosis of cells, and thus producing an antiviral effect. STING pathway can regulate the innate immune recognition of immunogenic tumors and promote the anti-tumor effect of interferons. IFN-γ plays an anti-tumor role in vivo through RAIL (tumomecrosis factor-related apoptosis-inducing ligand) and promotes tumor cell apoptosis.

cGAMP is a key stimulator of the innate immune response and an endogenous activator of STING, and has immune antitumor effects. Ectonucleotide pyrophosphatase/phosphodiesterase-1 (ENPP1) is a major cGAMP hydrolase that can degrade cGAMP. ENPP1 protein has a wide range of specificities, and can cleave a variety of substrates including phosphodiester bonds of nucleotides and nucleotide sugars and pyrophosphate bonds of nucleotides and nucleotide sugars. The protein can hydrolyze nucleoside 5′-triphosphate into its corresponding monophosphates, and can also hydrolyze diadenosine polyphosphate. ENPP1 inhibitor compounds can block the degradation of cGAMP extracellularly.

Currently, there are many studies focused on such action mechanisms, but no ENPP1 inhibitors have been found to be marketed, and therefore there is an urgent need to develop an effective ENPP1 inhibitor for use in clinical patients.

### SUMMARY

An object of the present application is to provide novel compounds having an ENPP1 inhibitory activity, a pharmaceutical composition comprising the compounds, and use of the compounds in the manufacture of a medicament for treating solid tumors.

The present application provides a compound represented by formula (I-b):

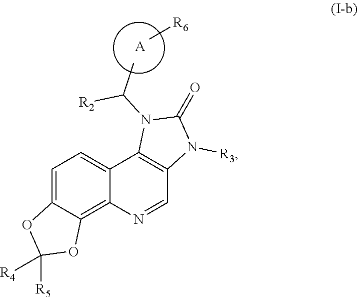

or a stereoisomer, a tautomer or a pharmaceutically acceptable salt thereof, wherein,
ring A is selected from the group consisting of aryl and 5- to 6-membered heteroaryl, wherein the aryl or heteroaryl is optionally substituted by 1 to 4 R₁;
R₁ is selected from the group consisting of hydrogen, halogen, C₁₋₄ alkyl, C₁₋₄ alkoxy, and aryl-C₁₋₄ alkoxy;
R₂ is selected from the group consisting of hydrogen and C₁₋₄ alkyl;
R₃ is selected from the group consisting of hydrogen, C₁₋₄ alkyl, C₃₋₄ cycloalkyl, 5- to 6-membered heterocycloalkyl, —C₁₋₄ alkylene-C₃₋₆ cycloalkyl, —C₁₋₄ alkylene-(5- to 6-membered heterocycloalkyl), —C₁₋₄ alkylene-CO—C₁₋₄ alkylene, and phenyl, which are optionally substituted by 1 to 3 R_{d}; wherein each carbon atom in the C₁₋₄ alkylene is optionally replaced by —N—, —O—, or —S—, and the S atom is optionally oxidized; R_{d} is selected from the group consisting of halogen, C₁₋₄ alkyl, and —NR_{da}R_{db}, wherein the C₃₋₆ cycloalkyl is optionally substituted by 1 to 4 Re; R_{da} and R_{db} are each independently selected from the group consisting of hydrogen and C₁₋₄ alkyl, and Re is selected from the group consisting of halogen and C₁₋₄ alkyl;
R₄ and R₅ are each independently selected from the group consisting of hydrogen and C₁₋₄ alkyl; and
R₆ is a hydrophilic group.

The present application provides a compound represented by formula (I-b):

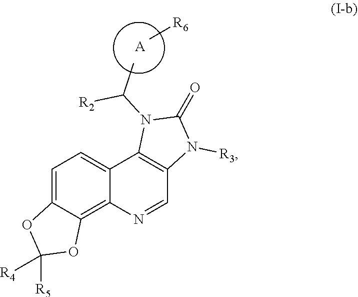

or a stereoisomer, a tautomer or a pharmaceutically acceptable salt thereof, wherein,
ring A is selected from the group consisting of aryl and 5- to 6-membered heteroaryl, wherein the aryl or heteroaryl is optionally substituted by 1 to 4 R₁;
R₁ is selected from the group consisting of hydrogen, halogen, C₁₋₄ alkyl, and C₁₋₄ alkoxy;
R₂ is selected from the group consisting of hydrogen and C₁₋₄ alkyl;
R₃ is selected from the group consisting of hydrogen, C₁₋₄ alkyl, C₁₋₆ cycloalkyl, 5- to 6-membered heterocycloalkyl, —C₁₋₄ alkylene-C₃₋₆ cycloalkyl, —C₁₋₄ alkylene-(5- to 6-membered heterocycloalkyl), —C₁₋₄ alkylene-CO—C₁₋₄ alkylene, and phenyl, which are optionally substituted by 1 to 3 R_{d}, wherein each carbon atom in the C₁₋₄ alkylene is optionally replaced by —N—, —O—, or —S—, and S atom is optionally oxidized; R_{d} is selected from the group consisting of halogen, C₁₋₄ alkyl, and —NR_{da}R_{db}, wherein the C₃₋₆ cycloalkyl is optionally substituted by 1 to 4 Re; R_{da} and R_{db} are each independently selected from the group consisting of hydrogen and C₁₋₄ alkyl, and Re is selected from the group consisting of halogen and C₁₋₄ alkyl;
R₄ and R₅ are each independently selected from the group consisting of hydrogen and C₁₋₄ alkyl; and
R₆ is a hydrophilic group.

The present application provides a compound represented by formula (I-b):

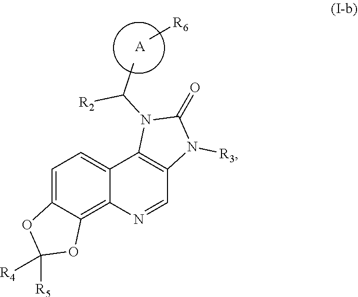

or a stereoisomer, a tautomer or a pharmaceutically acceptable salt thereof, wherein,
ring A is selected from the group consisting of aryl and 5- to 6-membered heteroaryl, wherein the aryl or heteroaryl is optionally substituted by 1 to 4 R₁;
R₁ is selected from the group consisting of hydrogen, halogen, and C₁₋₄ alkyl;
R₂ is selected from the group consisting of hydrogen and C₁₋₄ alkyl;
R³ is selected from the group consisting of hydrogen, C₁₋₄ alkyl, C₃₋₆ cycloalkyl, 5- to 6-membered heterocycloalkyl, —C₁₋₄ alkylene-C₃₋₆ cycloalkyl, and —C₁₋₄ alkylene-(5- to 6-membered heterocycloalkyl), which are optionally substituted by 1 to 3 R_{d}; wherein each carbon atom in the C₁₋₄ alkylene is optionally replaced by —N—, —O—, or —S—, and the S atom is optionally oxidized; R_{d} is selected from the group consisting of halogen, C₁₋₄ alkyl, and —NR_{da}R_{db}, wherein the C₃₋₆ cycloalkyl is optionally substituted by 1 to 4 Re; R_{da} and R_{db} are each independently selected from the group consisting of hydrogen and C₁₋₄ alkyl, and Re is selected from the group consisting of halogen and C₁₋₄ alkyl;
R₄ and R₅ are each independently selected from the group consisting of hydrogen and C₁₋₄ alkyl; and
R_{b} is a hydrophilic group.

The present application also provides a compound represented by formula (I-a):

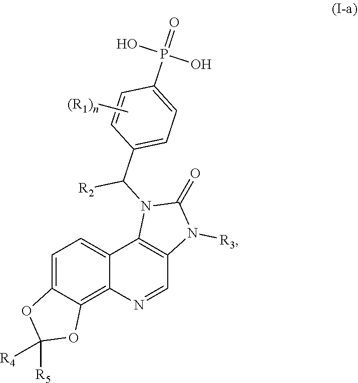

or a stereoisomer, a tautomer or a pharmaceutically acceptable salt thereof, wherein,
R₁ is selected from the group consisting of hydrogen, halogen, C₁₋₄ alkyl, C₁₋₄ alkoxy, and aryl-C₁₋₄ alkoxy;
R₂ is selected from the group consisting of hydrogen and C₁₋₄ alkyl;
R₃ is selected from the group consisting of hydrogen. C₁₋₄ alkyl, —C₁₋₄ alkylene-CO—C₁₋₄ alkylene, and phenyl, which are optionally substituted by 1 to 2 R_{d}; wherein each carbon atom in the C₁₋₄ alkylene is optionally replaced by —N—, —O—, or —S—; R_{d} is selected from the group consisting of halogen, C₁₋₄ alkyl, and —NR_{da}R_{db}; R_{da}, and R_{db} are each independently selected from the group consisting of hydrogen and C₁₋₄ alkyl;
R₄ and R₅ are each independently selected from the group consisting of hydrogen and C₁₋₄ alkyl; and
n is 1, 2, 3 or 4.

The present application also provides a compound represented by formula (I-a):

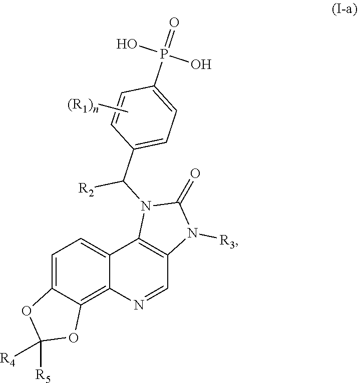

or a stereoisomer, a tautomer or a pharmaceutically acceptable salt thereof, wherein,
R₁ is selected from the group consisting of hydrogen, halogen. C₁₋₄ alkyl, and C₁₋₄ alkoxy;
R₂ is selected from the group consisting of hydrogen and C₁₋₄ alkyl;
R₃ is selected from the group consisting of hydrogen. C₁₋₄ alkyl, —C₁₋₄ alkylene-CO—C₁₋₄ alkylene, and phenyl, which are optionally substituted by 1 to 2 R_{d}; wherein each carbon atom in the C₁₋₄ alkylene is optionally replaced by —N—, —O—, or —S—; R_{d} is selected from the group consisting of halogen, C₁₋₄ alkyl, and —NR_{da}R_{db}; R_{da} and R_{db} are each independently selected from the group consisting of hydrogen and C₁₋₄ alkyl;
R₄ and R₅ are each independently selected from the group consisting of hydrogen and —C₁₋₄ alkyl; and
n is 1, 2, 3 or 4.

The present application also provides a compound represented by formula (I-a):

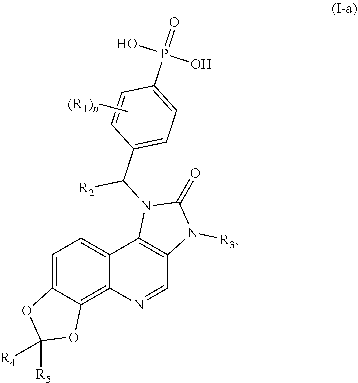

or a stereoisomer, a tautomer or a pharmaceutically acceptable salt thereof, wherein,
R₁ is selected from the group consisting of hydrogen, halogen, and C₁₋₄ alkyl;
R₂ is selected from the group consisting of hydrogen and C₁₋₄ alkyl;
R₃ is selected from the group consisting of hydrogen, and C₁₋₄ alkyl optionally substituted by 1 to 2 R_{d}, wherein R_{d} is selected from the group consisting of halogen and C₁₋₄ alkyl;
R₄ and R₅ are each independently selected from the group consisting of hydrogen and —C₁₋₄ alkyl; and
n is 1, 2, 3 or 4.

In some embodiments of the present application, ring A is selected from the group consisting of phenyl,

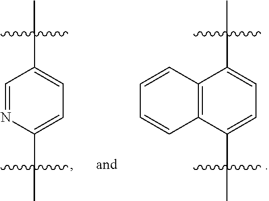 [SMILES: *c1ccc(*)nc1 ; *c1ccc(*)c2ccccc12]

In some embodiments of the present application, R₁ is selected from the group consisting of fluorine, chlorine, bromine, and methyl.

In some embodiments of the present application, R₁ is methoxy.

In some embodiments of the present application, R₁ is benzyloxy.

In some embodiments of the present application, R₁ is preferably fluorine.

In some embodiments of the present application, R₂ is selected from the group consisting of hydrogen and methyl.

In some embodiments of the present application, R₂ is preferably hydrogen.

In some embodiments of the present application, R₃ is selected from the group consisting of hydrogen, ethyl,

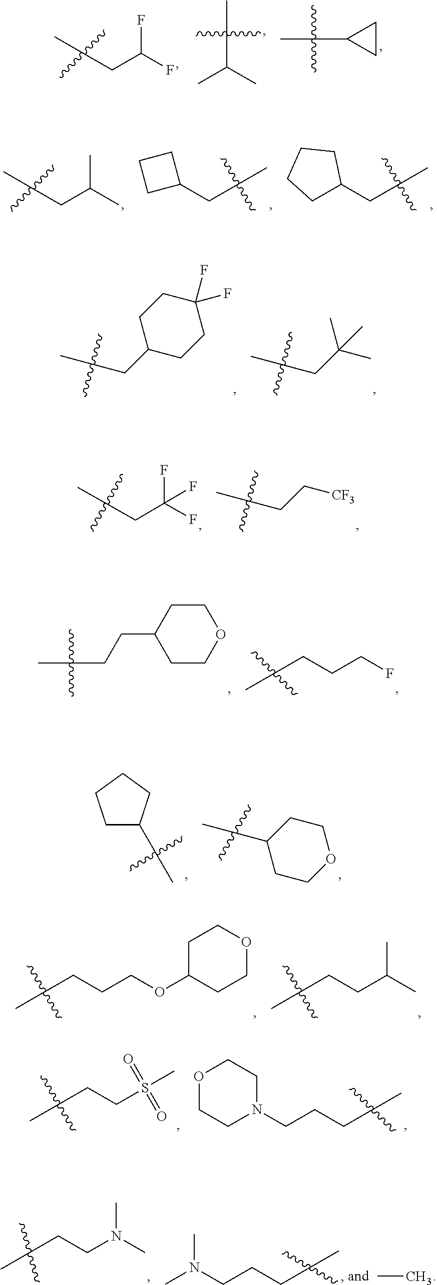

In some embodiments of the present application, R₃ is selected from the group consisting of

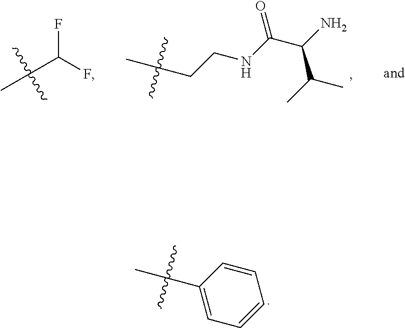

In some embodiments of the present application, R₃ is preferably hydrogen.

In some embodiments of the present application, R₄ is methyl.

In some embodiments of the present application, R₅ is methyl.

In some embodiments of the resent application, R₆ is selected from the group consisting of

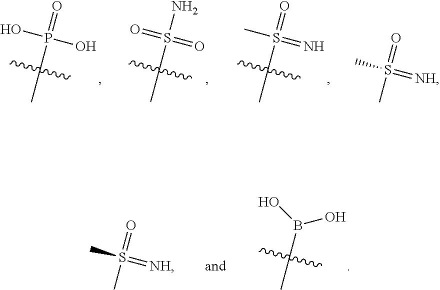

In some embodiments of the resent application, R₆ is preferably

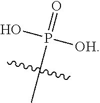 [SMILES: *P(=O)(O)O]

The present application also provides the following compounds, which are selected from the group consisting of:

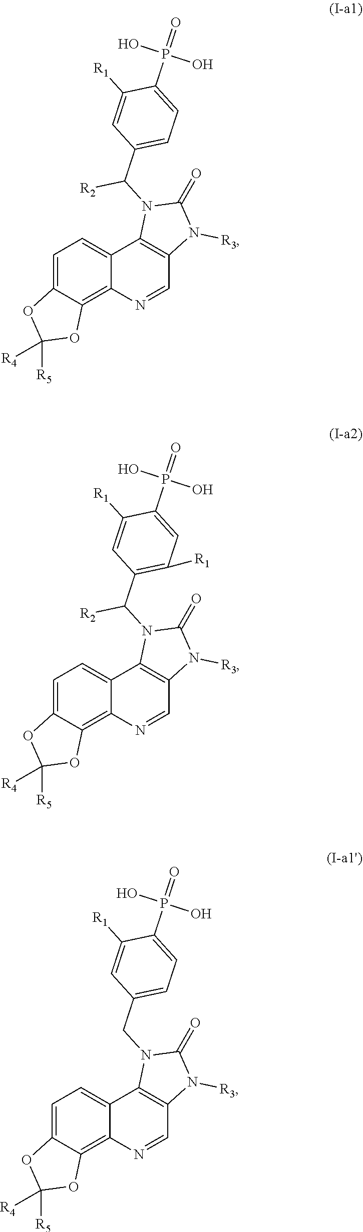

or a stereoisomer, a tautomer, or a pharmaceutically acceptable salt thereof,
wherein R₁, R₂, R₃, R₄, and R₅ are as defined above.

The present application also provides the following compounds, which are selected from the group consisting of:

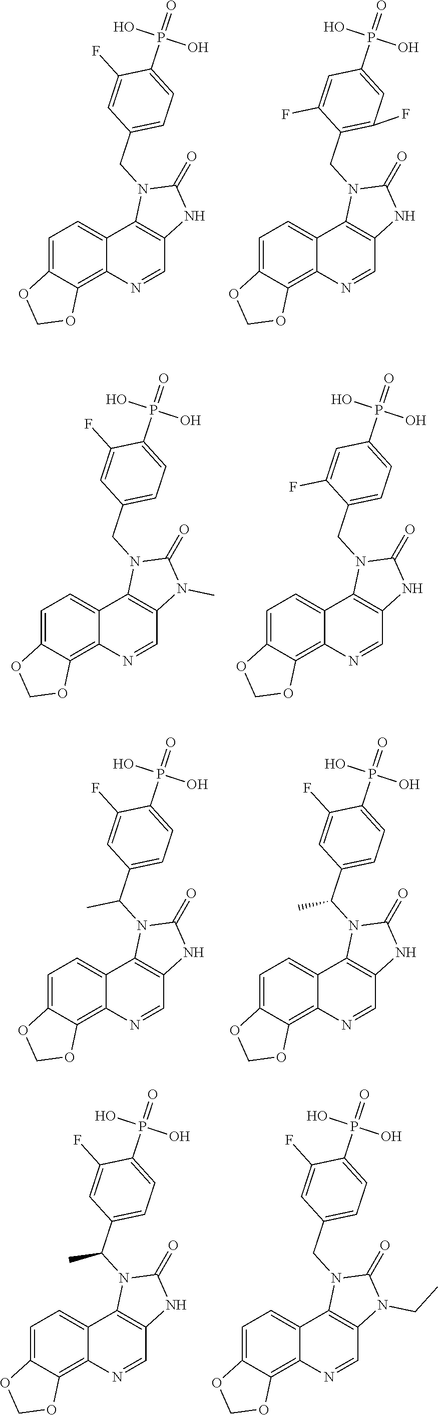

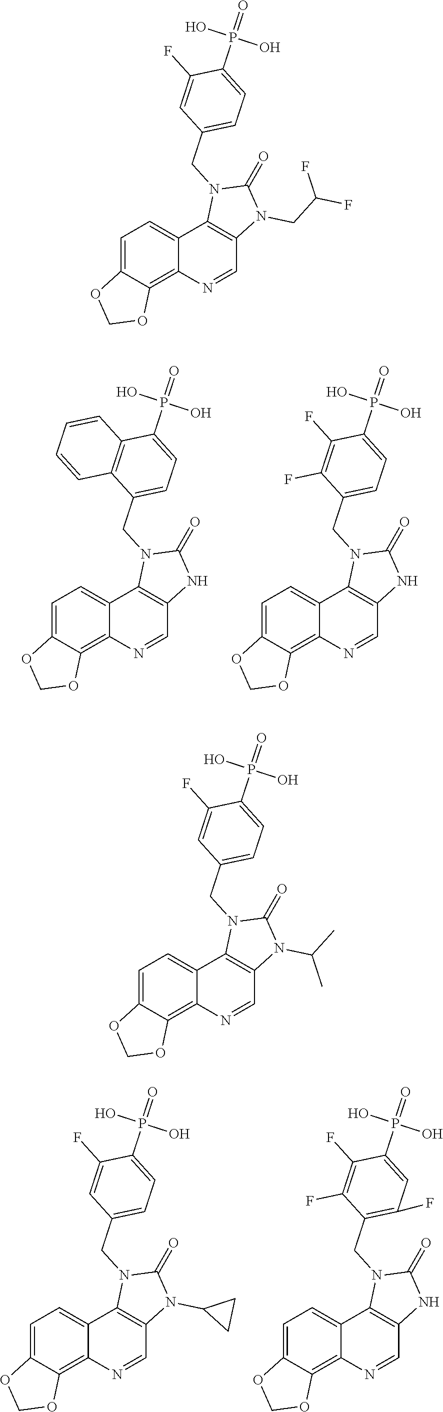

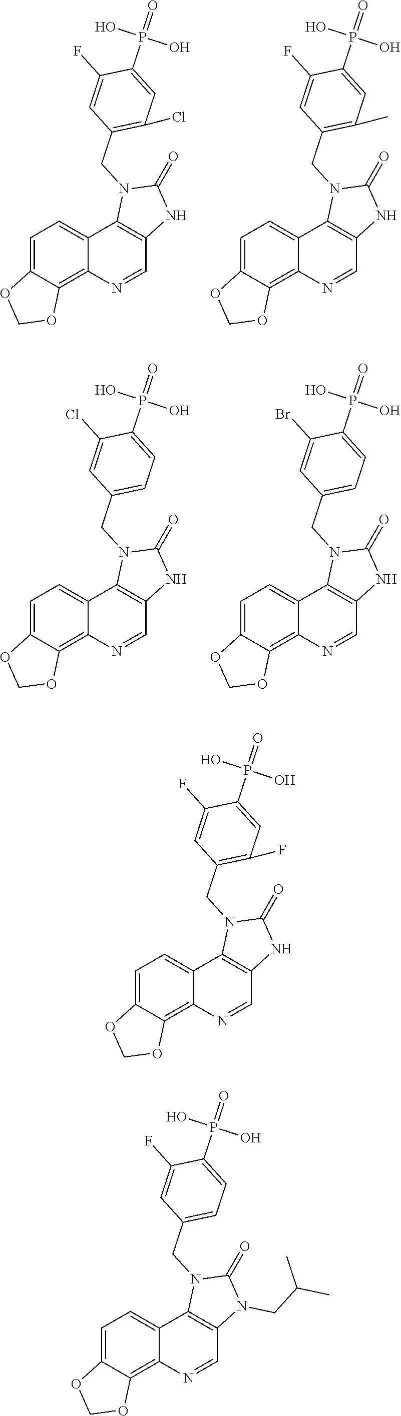 [SMILES: O=c1[nH]c2cnc3c4c(ccc3c2n1Cc1cc(F)c(P(=O)(O)O)cc1Cl)OCO4 ; Cc1cc(P(=O)(O)O)c(F)cc1Cn1c(=O)[nH]c2cnc3c4c(ccc3c21)OCO4 ; O=c1[nH]c2cnc3c4c(ccc3c2n1Cc1ccc(P(=O)(O)O)c(Cl)c1)OCO4 ; O=c1[nH]c2cnc3c4c(ccc3c2n1Cc1ccc(P(=O)(O)O)c(Br)c1)OCO4 ; O=c1[nH]c2cnc3c4c(ccc3c2n1Cc1cc(F)c(P(=O)(O)O)cc1F)OCO4 ; CC(C)Cn1c(=O)n(Cc2ccc(P(=O)(O)O)c(F)c2)c2c3ccc4c(c3ncc21)OCO4]

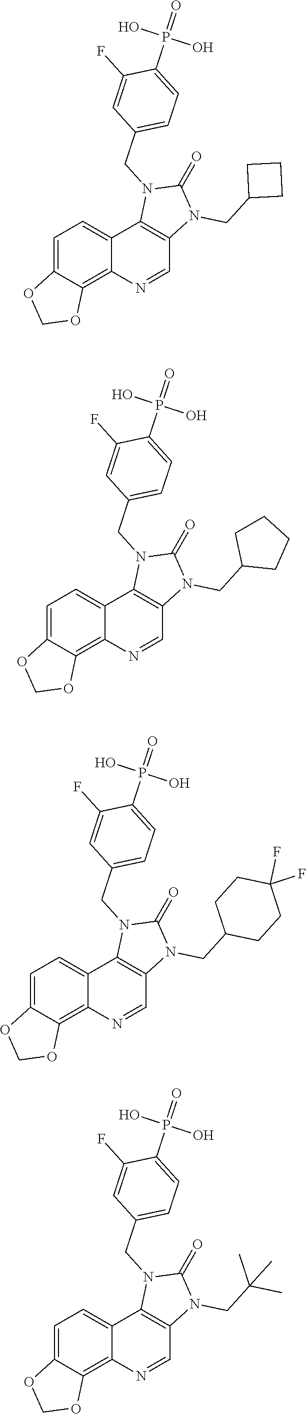 [SMILES: O=c1n(CC2CCC2)c2cnc3c4c(ccc3c2n1Cc1ccc(P(=O)(O)O)c(F)c1)OCO4 ; O=c1n(CC2CCCC2)c2cnc3c4c(ccc3c2n1Cc1ccc(P(=O)(O)O)c(F)c1)OCO4 ; O=c1n(CC2CCC(F)(F)CC2)c2cnc3c4c(ccc3c2n1Cc1ccc(P(=O)(O)O)c(F)c1)OCO4 ; CC(C)(C)Cn1c(=O)n(Cc2ccc(P(=O)(O)O)c(F)c2)c2c3ccc4c(c3ncc21)OCO4]

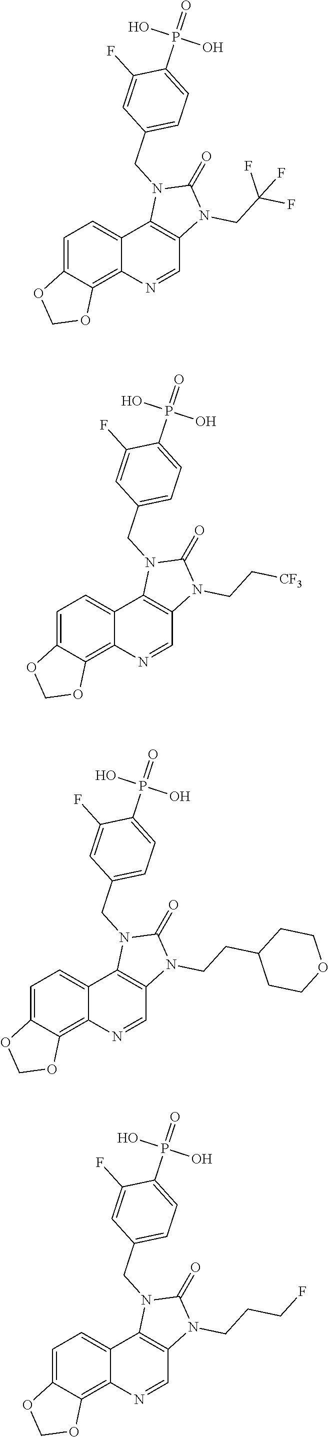 [SMILES: O=c1n(CC(F)(F)F)c2cnc3c4c(ccc3c2n1Cc1ccc(P(=O)(O)O)c(F)c1)OCO4 ; O=c1n(CCC(F)(F)F)c2cnc3c4c(ccc3c2n1Cc1ccc(P(=O)(O)O)c(F)c1)OCO4 ; O=c1n(CCC2CCOCC2)c2cnc3c4c(ccc3c2n1Cc1ccc(P(=O)(O)O)c(F)c1)OCO4 ; O=c1n(CCCF)c2cnc3c4c(ccc3c2n1Cc1ccc(P(=O)(O)O)c(F)c1)OCO4]

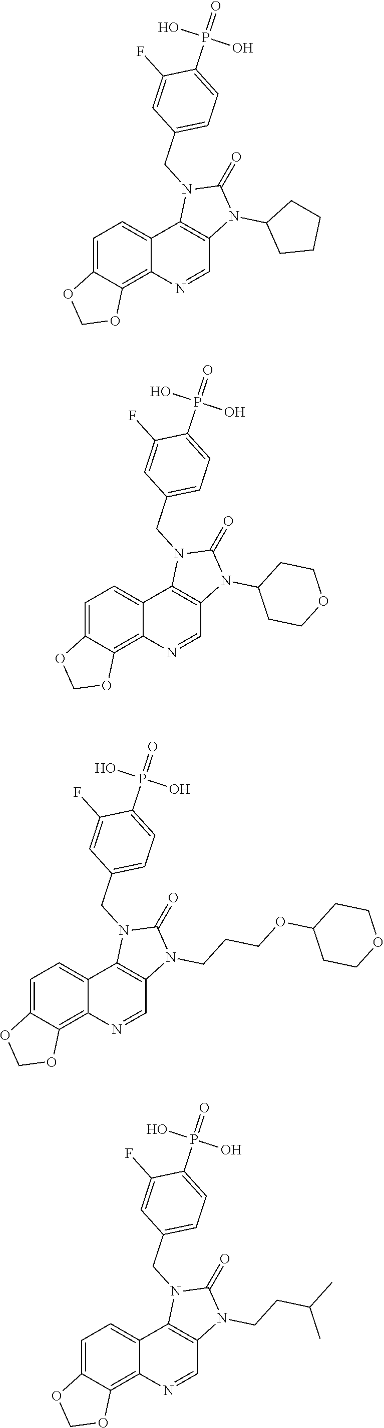 [SMILES: O=c1n(Cc2ccc(P(=O)(O)O)c(F)c2)c2c3ccc4c(c3ncc2n1C1CCCC1)OCO4 ; O=c1n(Cc2ccc(P(=O)(O)O)c(F)c2)c2c3ccc4c(c3ncc2n1C1CCOCC1)OCO4 ; O=c1n(CCCOC2CCOCC2)c2cnc3c4c(ccc3c2n1Cc1ccc(P(=O)(O)O)c(F)c1)OCO4 ; CC(C)CCn1c(=O)n(Cc2ccc(P(=O)(O)O)c(F)c2)c2c3ccc4c(c3ncc21)OCO4]

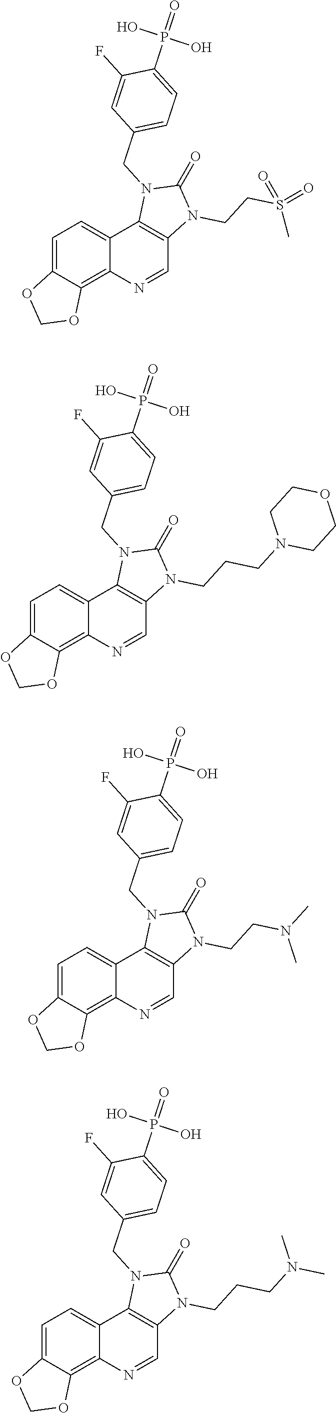 [SMILES: CS(=O)(=O)CCn1c(=O)n(Cc2ccc(P(=O)(O)O)c(F)c2)c2c3ccc4c(c3ncc21)OCO4 ; O=c1n(CCCN2CCOCC2)c2cnc3c4c(ccc3c2n1Cc1ccc(P(=O)(O)O)c(F)c1)OCO4 ; CN(C)CCn1c(=O)n(Cc2ccc(P(=O)(O)O)c(F)c2)c2c3ccc4c(c3ncc21)OCO4 ; CN(C)CCCn1c(=O)n(Cc2ccc(P(=O)(O)O)c(F)c2)c2c3ccc4c(c3ncc21)OCO4]

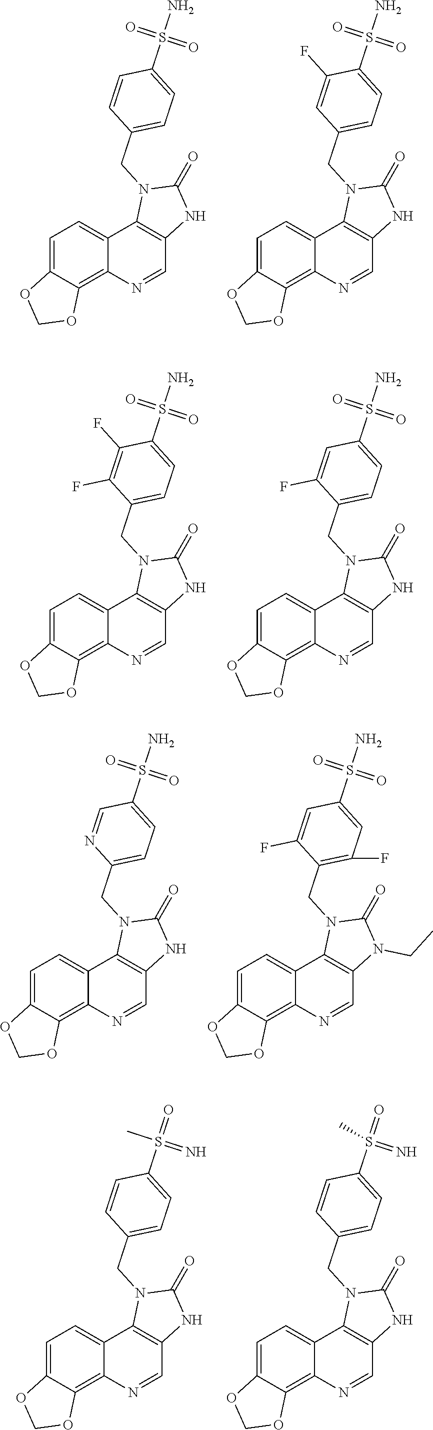

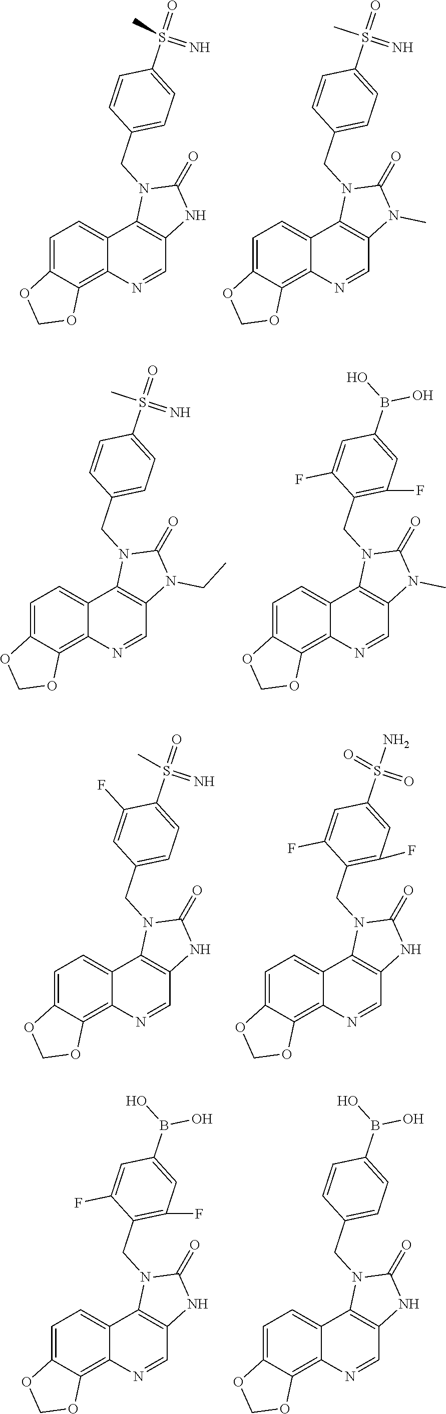

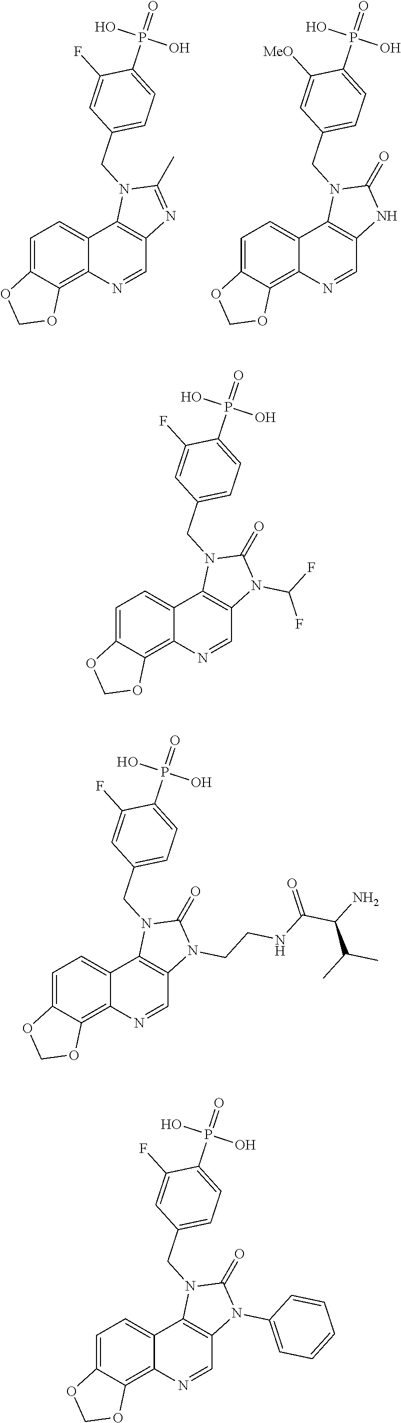 [SMILES: Cc1nc2cnc3c4c(ccc3c2n1Cc1ccc(P(=O)(O)O)c(F)c1)OCO4 ; COc1cc(Cn2c(=O)[nH]c3cnc4c5c(ccc4c32)OCO5)ccc1P(=O)(O)O ; O=c1n(Cc2ccc(P(=O)(O)O)c(F)c2)c2c3ccc4c(c3ncc2n1C(F)F)OCO4 ; CC(C)[C@H](N)C(=O)NCCn1c(=O)n(Cc2ccc(P(=O)(O)O)c(F)c2)c2c3ccc4c(c3ncc21)OCO4 ; O=c1n(Cc2ccc(P(=O)(O)O)c(F)c2)c2c3ccc4c(c3ncc2n1-c1ccccc1)OCO4]

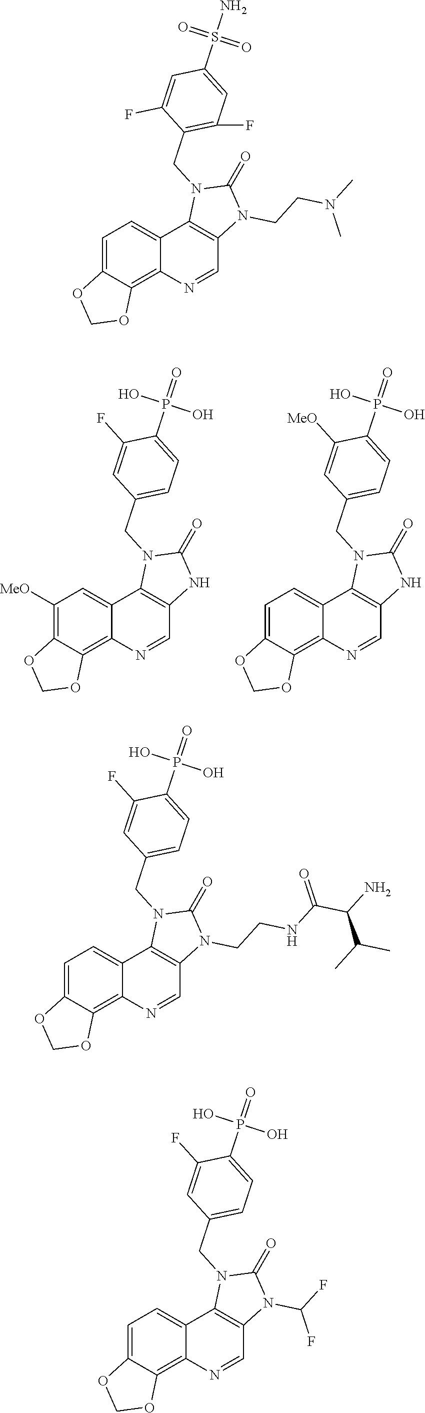 [SMILES: CN(C)CCn1c(=O)n(Cc2c(F)cc(S(N)(=O)=O)cc2F)c2c3ccc4c(c3ncc21)OCO4 ; COc1cc2c(ncc3[nH]c(=O)n(Cc4ccc(P(=O)(O)O)c(F)c4)c32)c2c1OCO2 ; COc1cc(Cn2c(=O)[nH]c3cnc4c5c(ccc4c32)OCO5)ccc1P(=O)(O)O ; CC(C)[C@H](N)C(=O)NCCn1c(=O)n(Cc2ccc(P(=O)(O)O)c(F)c2)c2c3ccc4c(c3ncc21)OCO4 ; O=c1n(Cc2ccc(P(=O)(O)O)c(F)c2)c2c3ccc4c(c3ncc2n1C(F)F)OCO4]

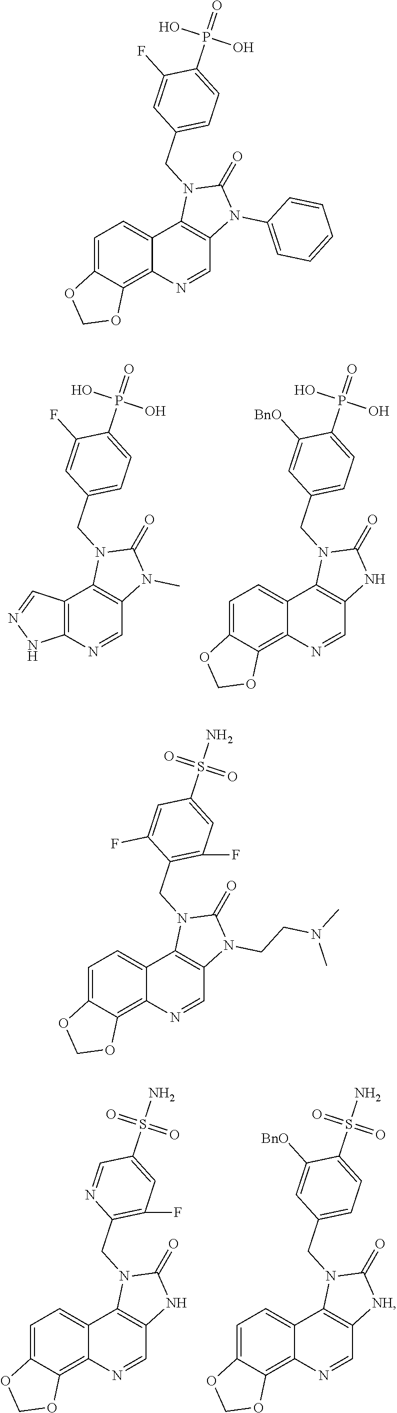 [SMILES: O=c1n(Cc2ccc(P(=O)(O)O)c(F)c2)c2c3ccc4c(c3ncc2n1-c1ccccc1)OCO4 ; Cn1c(=O)n(Cc2ccc(P(=O)(O)O)c(F)c2)c2c3cn[nH]c3ncc21 ; O=c1[nH]c2cnc3c4c(ccc3c2n1Cc1ccc(P(=O)(O)O)c(OCc2ccccc2)c1)OCO4 ; CN(C)CCn1c(=O)n(Cc2c(F)cc(S(N)(=O)=O)cc2F)c2c3ccc4c(c3ncc21)OCO4 ; NS(=O)(=O)c1cnc(Cn2c(=O)[nH]c3cnc4c5c(ccc4c32)OCO5)c(F)c1 ; NS(=O)(=O)c1ccc(Cn2c(=O)[nH]c3cnc4c5c(ccc4c32)OCO5)cc1OCc1ccccc1]

or a stereoisomer, a tautomer, or a pharmaceutically acceptable salt thereof.

The present application also provides a pharmaceutical composition comprising a “prophylactically or therapeutically effective amount” of the above compound or the stereoisomer, tautomer or pharmaceutically acceptable salt thereof, and a pharmaceutically acceptable carrier.

The present application also provides use of the compound or the stereoisomer, tautomer or pharmaceutically acceptable salt thereof, or the pharmaceutical composition, in the manufacture of a medicament for treating solid tumor.

The present application also provides use of the compound or the stereoisomer, tautomer, or pharmaceutically acceptable salt thereof in the treatment of a solid tumor.

In some embodiments of the present application, the solid tumor is an ENPP1 mediated solid tumor.

In some embodiments of the present application, the solid tumor in the above-described uses comprises a solid tumor of bile duct, bone, bladder, central nervous system, breast, colorectum, stomach, head and neck, liver, lung, neuron, esophagus, ovary, pancreas, prostate, kidney, skin, testis, thyroid, uterus, vulva, and the like.

### Technical Effect

The compound of the present application has a significant enzymatic inhibitory activity and can be used in the treatment of solid tumors.

### Description and Definition

Unless otherwise stated, the following terms and phrases used herein are intended to have the following meanings. A particular term or phrase should not be considered indefinite or unclear without a specific definition, but should be understood in its ordinary meaning.

The term “pharmaceutically acceptable” refers to the compounds, materials, compositions and/or dosage forms which, within a reasonable medical judgment, are suitable for use in contact with human and animal tissues without excessive toxicity, irritation, anaphylaxis or other problems or complications and with a reasonable benefit/risk ratio.

The term “pharmaceutically acceptable salt” refers to a derivative prepared from the compound of the present application and a relatively non-toxic acid or base. These salts can be prepared during compound synthesis, separation, or purification, or are prepared separately by reacting the purified compound in free form with a suitable acid or base. When a compound contains a relatively acidic functional group, it reacts with an alkali metal hydroxide, an alkaline earth metal hydroxide, or an organic amine to obtain an alkali addition salt, including salts of a cation based on the alkali metal or alkaline earth metal and a cation based on non-toxic ammonium, quaternary ammonium and amine, as well as salts of amino acids, and the like. When a compound contains a relatively basic functional group, it reacts with an organic or inorganic acid to obtain an acid addition salt.

The term “optionally substituted” means that the subject may be or may not substituted, and the type and number of substituents may be arbitrary on a chemically realizable basis, unless otherwise specified. For example, the phrase “C₁₋₄ alkyl optionally substituted by 1 to 2 R_{d}” means that it may be substituted by one or two R_{d} and may not be substituted.

The isomer of the compound of the present application includes a geometric isomer and a stereoisomer, such as a cis-trans isomer, an enantiomer, a diastereomer, a racemic mixture, and other mixtures thereof, all of which fall within the scope of the present application.

The term “enantiomer” refers to either of a pair of stereoisomers that are mirror images of each other.

The term “tautomer” refers to a type of functional group isomer which has different hydrogen attachment points through one or more double bond displacements, e.g., a ketone and its enol form are keto-enol tautomers.

The term “diastereomer” refers to either of stereoisomers of a molecule having two or more chiral centers, and the stereoisomers are not mirror images of each other.

The term “cis-trans isomer” refers to a configuration of a molecule in which a double bond or a single bond of ring carbon atoms cannot rotate freely.

Unless otherwise indicated, the absolute configuration of a stereo-center is indicated by a bond of wedge-shaped solid line [symbol] and a bond of wedge-shaped dashed line [symbol] , and the relative configuration of a stereo-center is indicated by a bond of straight-shaped solid line

[symbol]

and a bond of straight-shaped dashed line [symbol] . For example,

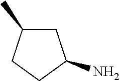

indicates methyl and amino groups located on the same side of the cyclopentane. The stereoisomers of the compound of the present application can be prepared by chiral synthesis or from chiral reagents or by other conventional techniques. For example, an enantiomer of the compound of the present application can be prepared by asymmetric catalytic technology or chiral additive derivative technology. Alternatively, through a chiral resolution technology, a single spatial configuration of the compound can be obtained from a mixture. Alternatively, it can be prepared directly from a chiral starting material. The separation of an optically pure compound in the present application is usually accomplished by preparative chromatography, and a chiral column is adopted to separate the chiral compound.

The absolute spatial configuration of the compound can be confirmed by means of conventional techniques in the art. For example, the absolute configuration of the compound can be confirmed through single-crystal X-ray diffraction, or can be confirmed by the chiral structure of raw materials and the reaction mechanism of asymmetric synthesis. Herein a compound marked as “absolute configuration not determined” is usually a single isomer separated from a racemic compound by chiral preparative SFC, which is then characterized and tested.

The term “pharmaceutically acceptable carrier” means a medium generally acceptable in the art for the delivery of a biologically active pharmaceutical agent to an animal, particularly a mammal. Depending on the method of administration and the nature of the dosage form, the pharmaceutically acceptable carrier includes, e.g., an adjuvant, an excipient or a vehicle, such as a diluent, a preservative, a filler, a flow regulator, a disintegrating agent, a wetting agent, an emulsifier, a suspending agent, a sweetener, a flavoring agent, a fragrant agent, an antimicrobial, an antifungal, a lubricant, and a dispersant. The pharmaceutically acceptable carrier is formulated depending on a large number of factors within the knowledge of those skilled in the art, comprising, but are not limited to: the type and nature of the active pharmaceutical agent formulated, the subject to which the composition containing the agent is to be administered, the intended route of administration of the composition, and the target therapeutic indication. The pharmaceutically acceptable carrier includes both aqueous and non-aqueous mediums and a variety of solid and semi-solid dosage forms. Such a carrier includes, in addition to an active pharmaceutical agent, many different ingredients and additives, and the additional ingredients included in a prescription for a variety of purpose (e.g., for stabilizing the active pharmaceutical agent, or as an adhesive, etc.) are well known to those skilled in the art.

The term “prophylactically or therapeutically effective amount” means an adequate amount of the compound of the present application or the pharmaceutically acceptable salt thereof which is suitable for the treatment of a disorder with a reasonable effect/risk ratio of any medical treatment and/or prevention. However, it should be understood that the total daily dosage of the compound represented by formula I of the present application or the pharmaceutically acceptable salt thereof and the composition shall be determined by an attending physician within the scope of reliable medical judgment. For any particular patient, the specific therapeutically effective dosage level must be determined based on multiple factors, including the disorder being treated and the seventy of the disorder; the activity of the specific compound used; the specific composition used, the age, weight, general health status, gender, and diet of a patient; the specific administration time, administration route, and excretion rate of the compound used; duration of treatment; medicaments used in combination or simultaneously with specific compounds used; and similar factors commonly known in the medical field. For example, the practice in the art is to start with a dose of a compound below the level required to achieve the desired therapeutic effect and gradually increase the dose until the desired effect is achieved.

Unless otherwise specified, the term “halogen” refers to a fluorine, chlorine, bromine or iodine atom.

Unless otherwise specified, the term “C₁₋₄ alkyl” is used to refer to a straight or branched saturated hydrocarbyl. Examples of alkyl include, but are not limited to, methyl, ethyl, n-propyl, isopropyl, n-butyl, isobutyl, etc.

Unless otherwise specified, the term “C₁₋₄ alkylene” is used to denote a divalent alkyl group having a specified number of 1 to 4 carbon atoms, including straight alkylene and branched alkylene; examples of which include, but are not limited to, —CH₂—, —CH₂CH₂—, —CH₂CH₂CH₂—, —CH₂CH₂CH₂CH₂—, —CH₂CH(CH₃)—, —CH₂CH(CH₃)CH₂—, —CH₂CH₂CH(CH₃)—, etc.

Unless otherwise specified, “C₃₋₆ cycloalkyl” refers to 3- to 6-membered substituted or unsubstituted monocycloalkyl; examples of monocycloalkyl include, but are not limited to, cyclopropyl, cyclobutyl, cyclopentyl, cyclohexyl, cycloheptyl, and cyclooctyl.

Unless otherwise specified, the term “aryl” refers to unsaturated, usually aromatic hydrocarbyl, which may be a monocyclic ring or a plurality of rings fused together. The aryl is preferably a C₅₋₁₀ aryl, more preferably a C₅₋₈ aryl, and most preferably a C₅₋₆ monocycloaryl. Examples of aryl include, but are not limited to, phenyl and naphthyl.

Unless otherwise specified, “5- to 6-membered heterocycloalkyl” refers to 5- to 6-membered substituted or unsubstituted monoheterocycloalkyl, and examples of such monoheterocycloalkyl include, but are not limited to, piperidinyl, piperazinyl, morpholinyl, tetrahydropyrrole, tetrahydrofuranyl, 3,4-dihydroxytetrahydrofuranyl, tetrahydrothienyl, tetrahydropyranyl, 1,3-dioxolane, 1,4-dioxane, etc.

Unless otherwise specified, the term “5- to 6-membered heteroaryl” refers to a 5- to 6-membered heteroaromatic ring in which C is replaced by 1, 2, or 3 nitrogen atoms; examples of which include, but are not limited to

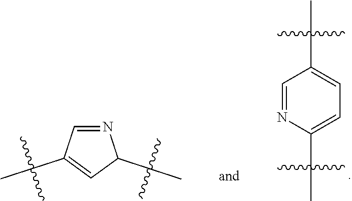 [SMILES: *C1=CC(*)N=C1 ; *c1ccc(*)nc1]

The compound of the present application may be prepared by a variety of synthetic methods known to those skilled in the art, including the specific embodiments illustrated below, embodiments resulting from combinations thereof with other methods of chemical synthesis, and equivalent substitutions known to those skilled in the art, with preferred embodiments including, but not limited to, examples of the present application.

The solvents used in the present application are commercially available.

In the present application, the compound structure is determined by nuclear magnetic resonance (NMR) or/and liquid chromatography-mass spectrometry (LC-MS). NMR chemical shift (8) is given in parts per million (ppm). NMR is performed with Bruker Neo 400M or Bruker Ascend 400 nuclear magnetic instrument, and solvent for measurement includes deuterated dimethyl sulfoxide (DMSO-d6), deuterated methanol (CD₃OD) and/or deuterated chloroform (CDCl₃), and the internal standard is tetramethylsilane (TMS).

Liquid chromatography-mass spectrometry, LC-MS, is performed with Agilent 1260-6125B single quadrupole mass spectrometer or Waters H-Class SQD2 mass spectrometer (ion source is electrospray ionization). HPLC is performed with Waters e2695-2998 or Waters ARC and Agilent 1260 or Agilent Poroshell HPH high-performance liquid chromatography.

Preparative HPLC is performed with Waters 2555-2489 (10 μm, ODS 250 cm×5 cm) or GILSON Trilution LC, and the chromatographic column is Welch XB-C18 column (5 μm, 21.2×150 mm).

Thin layer chromatography silica gel plate is performed with GF254 silica gel plate from Yantai Jiangyou Silica gel Development Co., Ltd. or GF254 silica gel plate from Rushan Sanpont New Materials Co., Ltd. The TLC specification is 0.15 mm to 0.20 mm, preparative, and 20×20 cm. Generally, 200 to 300 mesh silica gel is used as the carrier for the column chromatography.

The compounds are named according to the conventional naming principles in the art or using ChemDraw® software, and commercially available compounds are named using the supplier's catalog name.

### DETAILED DESCRIPTION OF THE INVENTION

The following provides a detailed description of the present application through examples, but does not imply any adverse limitations to the present application. The compound of the present application may be prepared by a variety of synthetic methods known to those skilled in the art, including the specific embodiments illustrated below, embodiments resulting from combinations thereof with other methods of chemical synthesis, and equivalent substitutions known to those skilled in the art, with preferred embodiments including, but not limited to, examples of the present application. For those skilled in the art, it will be apparent to make various changes and improvements to the specific embodiments of the present application without departing from the spirit and scope of the present application.

### I. Preparation Method

### Example 1: (2-fluoro-4-((7-oxo-6,7-dihydro-8H-[1,3]dioxolo[4,5-h]imidazo[4,5-c]quinolin-8-yl)methyl)phenyl)phosphonic add

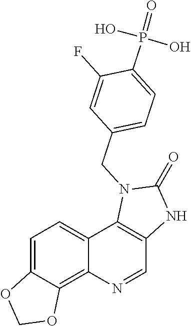 [SMILES: O=c1[nH]c2cnc3c4c(ccc3c2n1Cc1ccc(P(=O)(O)O)c(F)c1)OCO4]

### Reaction Route:

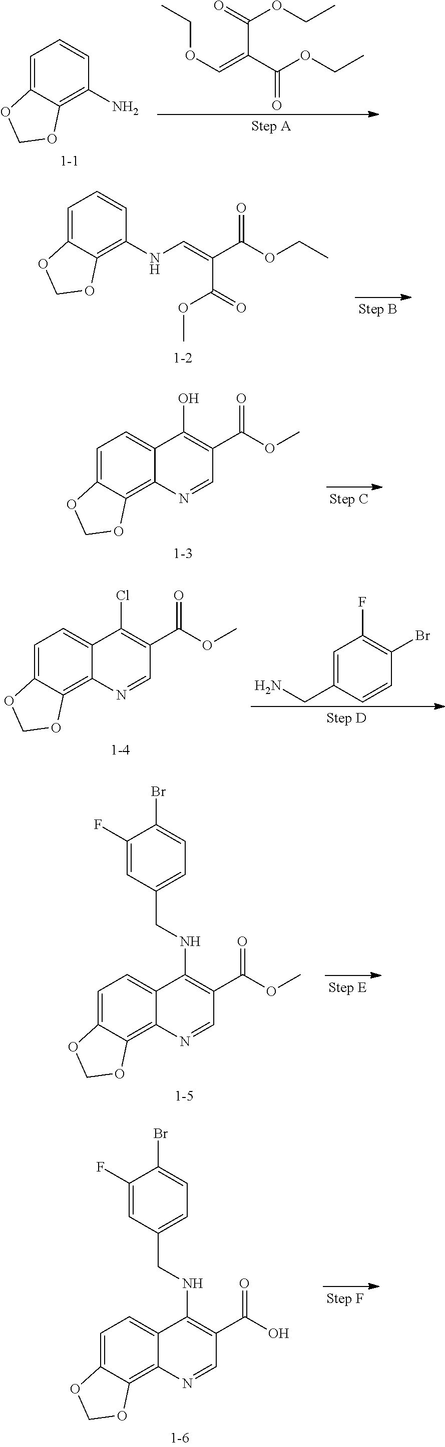

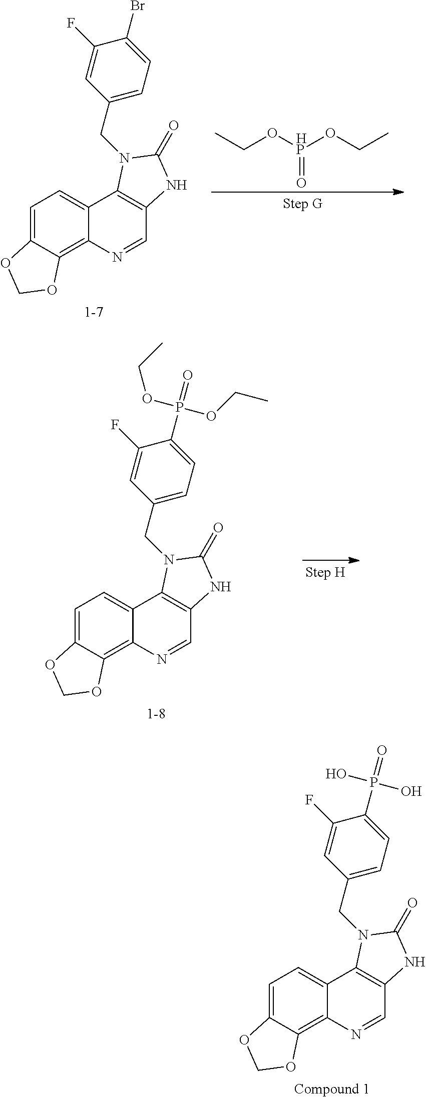

Step A: Benzo[d][1,3]dioxin-4-amine (1.37 go 10.0 mmol), diethyl 2-(ethoxymethylene)malonate (2.38 g, 11.0 mmol) were dissolved in acetonitrile (15 mL) at room temperature. Subsequently, triethylamine (2.8 mL, 20.0 mmol) was added to the resulting mixed reaction solution at room temperature, and then the mixture was stirred at room temperature for 33 h. After the depletion of raw material as monitored by TLC, the reaction solution was concentrated under reduced pressure, then petroleum ether/ethyl acetate (50 mL/5 mL) was added to the concentrated residue, and then the mixture was filtered. The resulting filter cake was further washed three times with petroleum ether/ethyl acetate (50 mL/5 mL) and the filter cake was dried to give 3.0 g of Intermediate 1-2.

MS (ESI) M/Z: 308.1 [M+H]⁺.

Step B: Intermediate 1-2 (2 g, 6.51 mmol) was dissolved in diphenyl ether (20 mL). Subsequently, the reaction system was stirred at 260′C for half an hour. The reaction solution was cooled to about 40° C., then petroleum ether (80 mL) was added to the reaction solution, and then the mixture was filtered. The resulting filter cake was continued to be washed three times with petroleum ether (20 mL) and the filter cake was dried to give 0.54 g of Intermediate 1-3.

MS (ESI) M/Z: 262.0 [M+H]⁺.

Step C: Intermediate 1-3 (0.7 g, 2.68 mmol) and N,N-diisopropylethylamine (0.3 mL) were dissolved in trichloromethane (70 mL) at room temperature. Subsequently, phosphorous oxychloride (2 mL) was slowly added to the solution at room temperature and the resulting mixed turbid reaction solution was stirred at 90° C. for 13 h, and the solution became clear and transparent. After the depletion of raw material as monitored by LCMS, the reaction solution was concentrated under reduced pressure, and 0.75 g of Intermediate 1-4 was obtained.

MS(ESI) M/Z: 280.0 [M+H]⁺.

Step D: Intermediate 1-4 (0.75 g, 2.68 mmol) and (4-bromo-3-fluorophenyl)methylamine (0.66 mg, 3.22 mmol) were dissolved in acetonitrile (11 mL) at room temperature. Subsequently, triethylamine (1.1 mL, 8.04 mmol) was added to the solution at room temperature and the resulting mixed reaction solution was stirred at 103° C. for 39 h. After the depletion of raw material as monitored by LCMS, petroleum ether/ethyl acetate (20 mL/20 mL) was added to the reaction solution, and the mixture was stirred at room temperature for 1 h and then filtered. The resulting filter cake was washed three times with petroleum ether/ethyl acetate (10 mL/10 mL) and the filter cake was dried to give 1.2 g of Intermediate 1-5.

MS (ESI) M/Z: 447.1 [M+H]⁺.

Step E: Intermediate 1-5 (1.2 g, 2.68 mmol) was dissolved in anhydrous tetrahydrofuran/water/methanol (16 mL/16 mL/4 mL) at room temperature. Subsequently, lithium hydroxide (0.64 mg, 26.8 mmol) was added to the solution at room temperature, and then the reaction solution was heated to room temperature and stirred at room temperature for 16 h. The LCMS monitoring showed that the raw material was not depleted. Sodium hydroxide (0.21 mg, 5.36 mmol) was added to the solution at room temperature, and the mixture was stirred for 23 h at 45° C. After the depletion of raw material as monitored by LCMS, the reaction solution was concentrated under reduced pressure, water (60 mL) was added to the resulting residue, and dilute hydrochloric acid (2 mol/L) was added to adjust the pH to about 4. The precipitated solid was filtered, then washed twice with water (10 mL) and dried to give 0.8 g of Intermediate 1-6.

MS (ESI) M/Z: 419.0 [M+H]⁺.

Step F: Intermediate 1-6 (0.8 g, 1.90 mmol) was dissolved in toluene (20 mL) at room temperature and diphenylphosphoryl azide (1.05 g, 3.81 mmol) and triethylamine (0.58 g, 5.72 mmol) were added thereto, and the resulting mixed reaction solution was stirred for 6 h at 120° C. After the depletion of raw material as monitored by LCMS, the resulting mixture was concentrated under reduced pressure, and the resulting residue was purified by silica gel column chromatography to give 0.5 g of Intermediate 1-7.

MS (ESI) M/Z: 416.1 [M+H]⁺.

Step G: Intermediate 1-7 (0.2 g, 0.48 mmol) was dissolved in N,N-dimethylformamide (5 mL) at room temperature. Subsequently, diethyl phosphate (0.4 g, 2.89 mmol), [1,1′-bis(diphenylphosphino)ferrocene]palladium(II) dichloride dichloromethane complex (0.078 g, 0.096 mmol), and triethylamine (0.29 g, 2.89 mmol) were added to the solution, and the resulting mixed reaction solution was stirred for 16 h at 100° C. After the depletion of raw material as monitored by LCMS, the mixture was stirred and poured into water, and the resultant was extracted with ethyl acetate (15 mL×2). The combined organic layer was dried over anhydrous sodium sulfate and concentrated to give a crude product, which was purified by silica gel column chromatography (dichloromethane/methanol=20/1, v/v) to give 70.0 mg of Intermediate 1-8.

MS (ESI) M/Z: 474.0 [M+H]⁺.

Step H: Trimethyl bromosilane (0.5 mL) was added dropwise to a solution of intermediate 1-8 (60.0 mg, 0.13 mmol) dissolved in acetonitrile (0.5 mL), and the mixture was stirred at room temperature for 16 h. After the depletion of raw material as monitored by LCMS, the mixture was concentrated to give a crude product, which was purified by preparative high performance liquid chromatography to give 33.0 mg of Compound 1.

MS (ESI) M/Z: 418.0 [M+H]⁺.

¹H NMR (400 MHz, DMSO-d6) δ 8.63 (s, 1H), 7.58-7.50 (m, 1H), 7.48 (d, J=9.1 Hz, 1H), 7.23 (d, J=9.0 Hz, 1H), 6.91 (d, J=7.8 Hz, 1H), 6.81 (dd, J=10.2, 4.3 Hz, 1H), 6.12 (s, 2H), 5.45 (s, 2H).

### Example 2: (3,5-difluoro-4-((7-oxo-6,7-dihydro-8H-[1,3]dioxolo[4,5-h]imidazo[4,5-c]quinolin-8-yl)methyl)phenyl)phosphonic acid

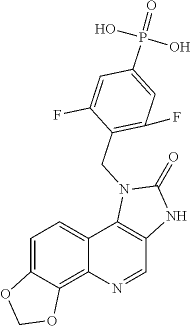 [SMILES: O=c1[nH]c2cnc3c4c(ccc3c2n1Cc1c(F)cc(P(=O)(O)O)cc1F)OCO4]

### Reaction Route:

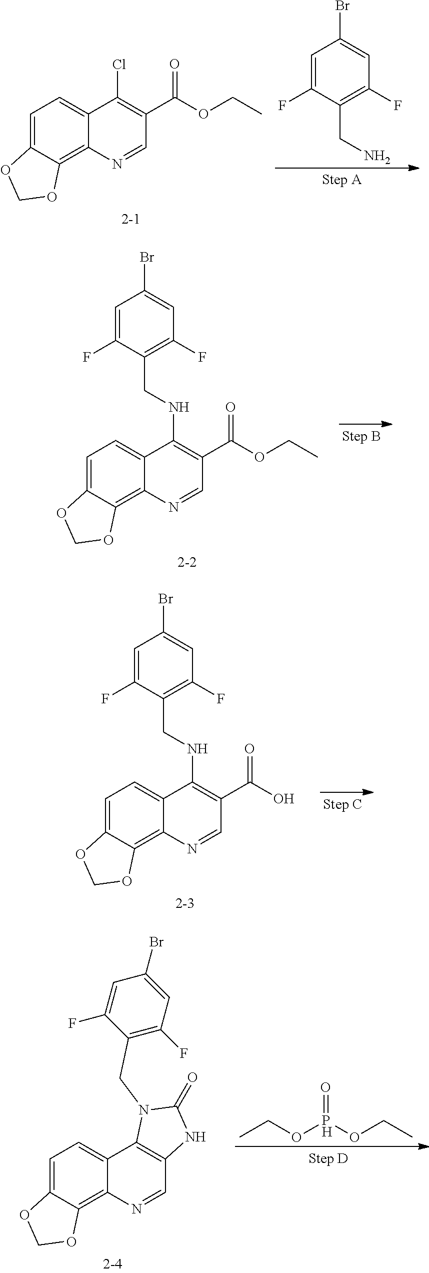

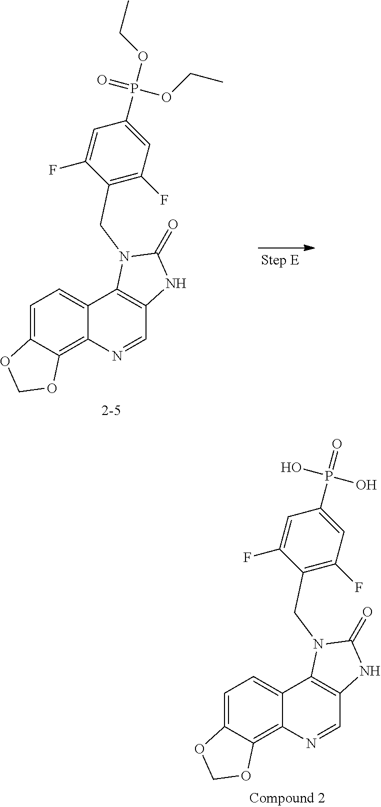 [SMILES: CCOP(=O)(OCC)c1cc(F)c(Cn2c(=O)[nH]c3cnc4c5c(ccc4c32)OCO5)c(F)c1 ; O=c1[nH]c2cnc3c4c(ccc3c2n1Cc1c(F)cc(P(=O)(O)O)cc1F)OCO4]

Step A: Ethyl 6-chloro-[1,3]dioxolo[4,5-h]quinolin-7-carboxylate (0.15 g, 0.54 mmol) and (4-bromo-2,6-difluorophenyl)methylamine (0.13 g, 3.22 mmol) were dissolved in acetonitrile (4 mL) at room temperature. Subsequently, triethylamine (0.15 mL, 1.072 mmol) was added to the solution at room temperature and the resulting mixed reaction solution was stirred at 100° C. for 18 h. After the depletion of raw material as monitored by LCMS, petroleum ether/ethyl acetate (13 mL/13 mL) was added to the reaction solution, and the mixture was stirred at room temperature for 1 h and then filtered. The resulting filter cake was washed three times with petroleum ether/ethyl acetate (10 mL/10 mL) and the filter cake was dried to give 0.2 g of Intermediate 2-2.

MS (ESI) M/Z: 464.8 [M+H]⁺.

Step B: Intermediate 2-2 (0.2 g, 0.43 mmol) was dissolved in anhydrous tetrahydrofuran/water/ethanol (4 mL/4 mL/4 mL) at room temperature. Subsequently, sodium hydroxide (0.12 g, 3.01 mmol) was added to the solution at room temperature, and then the reaction solution was stirred at 40° C. for 26 h. After the depletion of raw material as monitored by LCMS, the reaction solution was concentrated under reduced pressure. Water (20 mL) was added to the resulting residue, and dilute hydrochloric acid (2 mol/L) was added to adjust the pH to 5. The precipitated solid was filtered and then washed twice with water (10 mL) and dried to give 0.1 g of Intermediate 2-3.

MS (ESI) M/Z: 439.1 [M+H]⁺.

Step C: Intermediate 2-3 (0.1 g, 0.23 mmol) was dissolved in toluene (2 mL) at room temperature and diphenylphosphoryl azide (0.13 g, 0.46 mmol) and triethylamine (69.4 mg, 0.69 mmol) were added thereto, and the resulting mixed reaction solution was stirred for 18 h at 120° C. After the depletion of raw material as monitored by LCMS, the resulting mixture was concentrated under reduced pressure, and the resulting residue was purified by silica gel column chromatography to give 90.0 mg of Intermediate 2-4.

MS (ESI) M/Z: 433.8 [M+H]⁺.

Step D: Intermediate 2-4 (90.0 mg, 0.21 mmol) was dissolved in N,N-dimethylformamide (3.5 mL) at room temperature. Subsequently, diethyl phosphite (85.8 mg, 0.62 mmol), [1,1′-bis(diphenylphosphino)ferrocene]palladium(II) dichloride dichloromethane complex (16.3 mg, 0.02 mmol), and triethylamine (83.6 mg, 0.83 mmol) were added to the solution, and the resulting mixed reaction solution was stirred for 3 h at 100° C. under microwave. After the depletion of raw material as monitored by LCMS, the mixture was stirred and poured into water (20 mL) and extracted with dichloromethane (50 mL). The combined organic layer was washed with saturated saline (20 mL×3), dried over anhydrous sodium sulfate, filtered and concentrated to give a crude product, which was purified by silica gel column chromatography to give 70.0 mg of Intermediate 2-5.

MS (ESI) M/Z: 491.8 [M+H]⁺.

Step E: Trimethyl bromosilane (1.5 mL) was added dropwise to a solution of Intermediate 2-5 (70.0 mg, 0.14 mmol) dissolved in acetonitrile (3 mL), and the mixture was stirred at room temperature for 22 h. After the depletion of raw material as monitored by LCMS, the mixture was concentrated to give a crude product, which was purified by preparative high performance liquid chromatography to give 16.7 mg of Compound 2.

MS (ESI) M/Z: 435.6 [M+H]⁺.

¹H NMR (400 MHz, DMSO-d6) δ 8.61 (s, 1H), 7.66 (d, J=8.9 Hz, 1H), 7.31 (d, J=9.0 Hz, 7H), 7.17-7.00 (m, 2H), 6.18 (s, 2H), 5.55 (s, 2H).

### Example 3: (2-fluoro-4-((6-methyl-7-oxo-6,7-dihydro-8H-[1,3]dioxolo[4,5-h]imidazo[4,5-c]quinolin-8-yl)methyl)phenyl)phosphonic acid

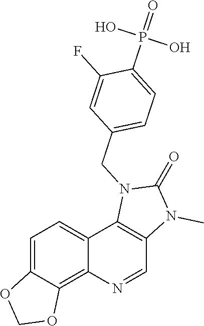 [SMILES: Cn1c(=O)n(Cc2ccc(P(=O)(O)O)c(F)c2)c2c3ccc4c(c3ncc21)OCO4]

### Reaction Route:

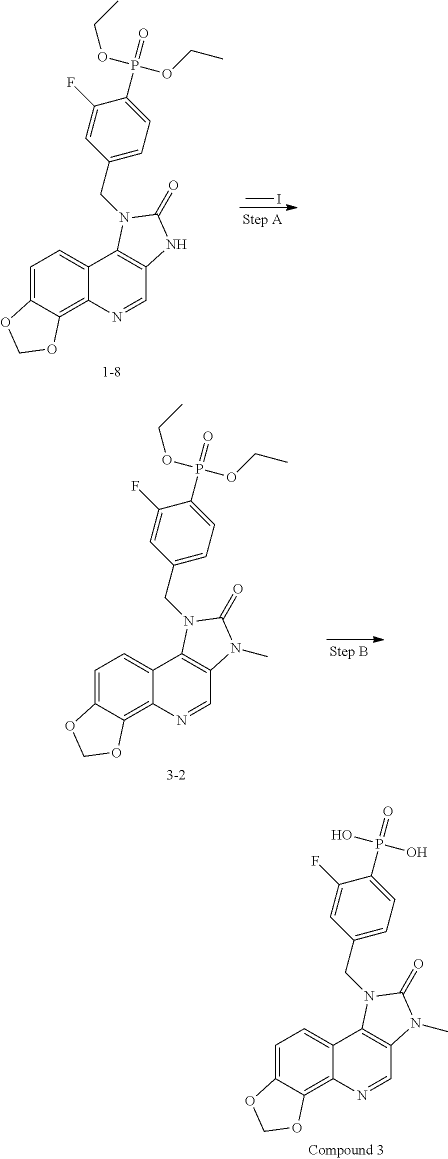

Step A: Sodium hydride (17.0 mg, 0.42 mmol) was added to a solution of Intermediate 1-8 (100.0 mg, 0.21 mmol) dissolved in N,N-dimethylformamide (3 mL) at a controlled temperature of 0° C. The mixture was stirred at 0° C. for 30 min and then methyl iodide (60.0 mg, 0.42 mmol) was added at 0° C., and the resulting mixed reaction solution was stirred at room temperature for 16 h. After the depletion of raw material as monitored by LCMS, ethyl acetate and water were added to the reaction solution for extraction, and the resulting aqueous phase was purified by preparative high-performance liquid chromatography (mobile phases: aqueous solution of formic acid in 1000 ppm and acetonitrile) to give 18.0 mg of Intermediate 3-2.

MS (ESI) M/Z: 460.3 [M+H]⁺

Step B: Trimethyl bromosilane (0.5 mL) was added dropwise to a solution of Intermediate 3-2 (18.0 mg, 0.039 mmol) dissolved in acetonitrile (1.5 mL), and the mixture was stirred at room temperature for 16 h. After the depletion of raw material as monitored by LCMS, the mixture was concentrated to give a crude product, which was purified by preparative high performance liquid chromatography (mobile phases: aqueous solution of formic acid in 1000 ppm and acetonitrile) to give 3.24 mg of Compound 3.

MS (ESI) M/Z: 431.9 [M+H]

¹H NMR (400 MHz, DMSO-d6) δ 8.90 (s, 1H), 7.63-7.55 (m, 1H), 7.48 (d, J=8.9 Hz, 1H), 7.29 (d, J=8.9 Hz, 1H), 7.16-7.00 (m, 2H), 6.21 (s, 2H), 5.56 (s, 2H), 3.59 (s, 3H).

### Example 4: (3-fluoro-4-((7-oxo-6,7-dihydro-8H-[1,3]dioxolo[4,5-h]imidazo[4,5-c]quinolin-8-yl)methyl)phenyl)phosphonic acid

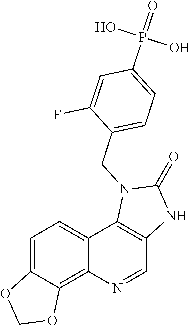 [SMILES: O=c1[nH]c2cnc3c4c(ccc3c2n1Cc1ccc(P(=O)(O)O)cc1F)OCO4]

### Reaction Route:

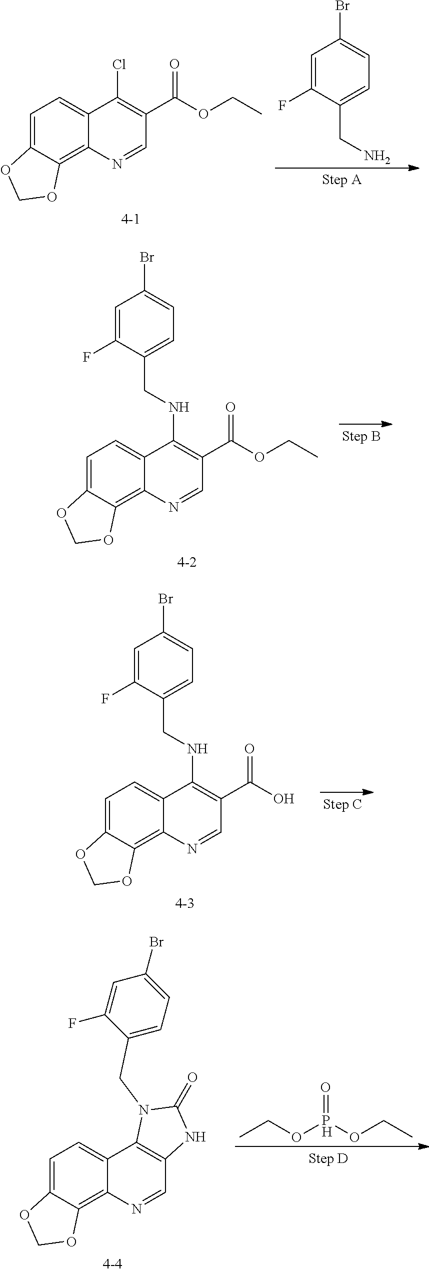

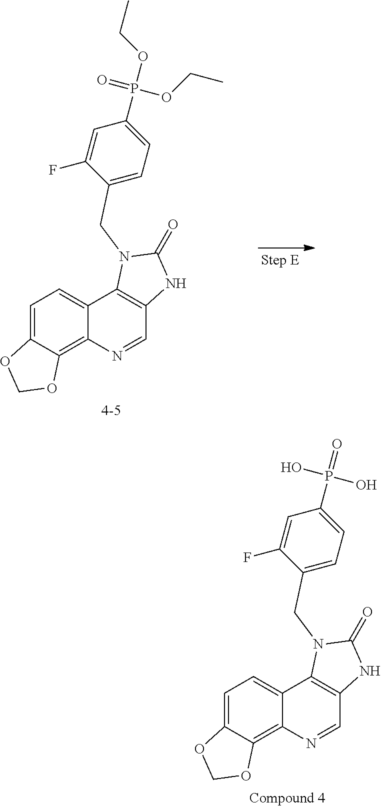 [SMILES: CCOP(=O)(OCC)c1ccc(Cn2c(=O)[nH]c3cnc4c5c(ccc4c32)OCO5)c(F)c1 ; O=c1[nH]c2cnc3c4c(ccc3c2n1Cc1ccc(P(=O)(O)O)cc1F)OCO4]

The synthesis procedure is the same as Example 1. (4-Bromo-3-fluorophenyl)methylamine was replaced by (4-bromo-2-fluorophenyl)methylamine (44 mg, 0.22 mmol), and 80.2 mg of Compound 4 was synthesized.

MS (ESI) M/Z: 417.6 [M+H]⁺.

¹H NMR (400 MHz, DMSO-d6) δ 8.66 (s, 1H), 7.44-7.31 (m, 2H), 7.31-7.20 (m, 2H), 6.89-6.75 (m, 1H), 6.19 (s, 2H), 5.48 (s, 2H).

Example 5: (S)-(2-fluoro-4-(1-(7-oxo-6,7-dihydro-8H-[1,3]dioxolo[4,5-h]imidazo[4,5-c]quinolin-8-yl)ethyl)phenyl)phosphonic acid and (R)-(2-fluoro-4-(1-(7-oxo-6,7-dihydro-8H-[1,3]dioxolo[4,5-h]imidazo[4,5-c]quinolin-8-yl)ethyl)phenyl)phosphonic acid

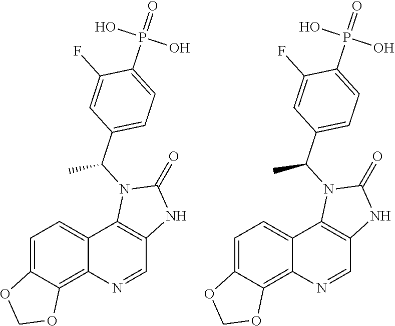 [SMILES: C[C@H](c1ccc(P(=O)(O)O)c(F)c1)n1c(=O)[nH]c2cnc3c4c(ccc3c21)OCO4 ; C[C@@H](c1ccc(P(=O)(O)O)c(F)c1)n1c(=O)[nH]c2cnc3c4c(ccc3c21)OCO4]

### Reaction Route:

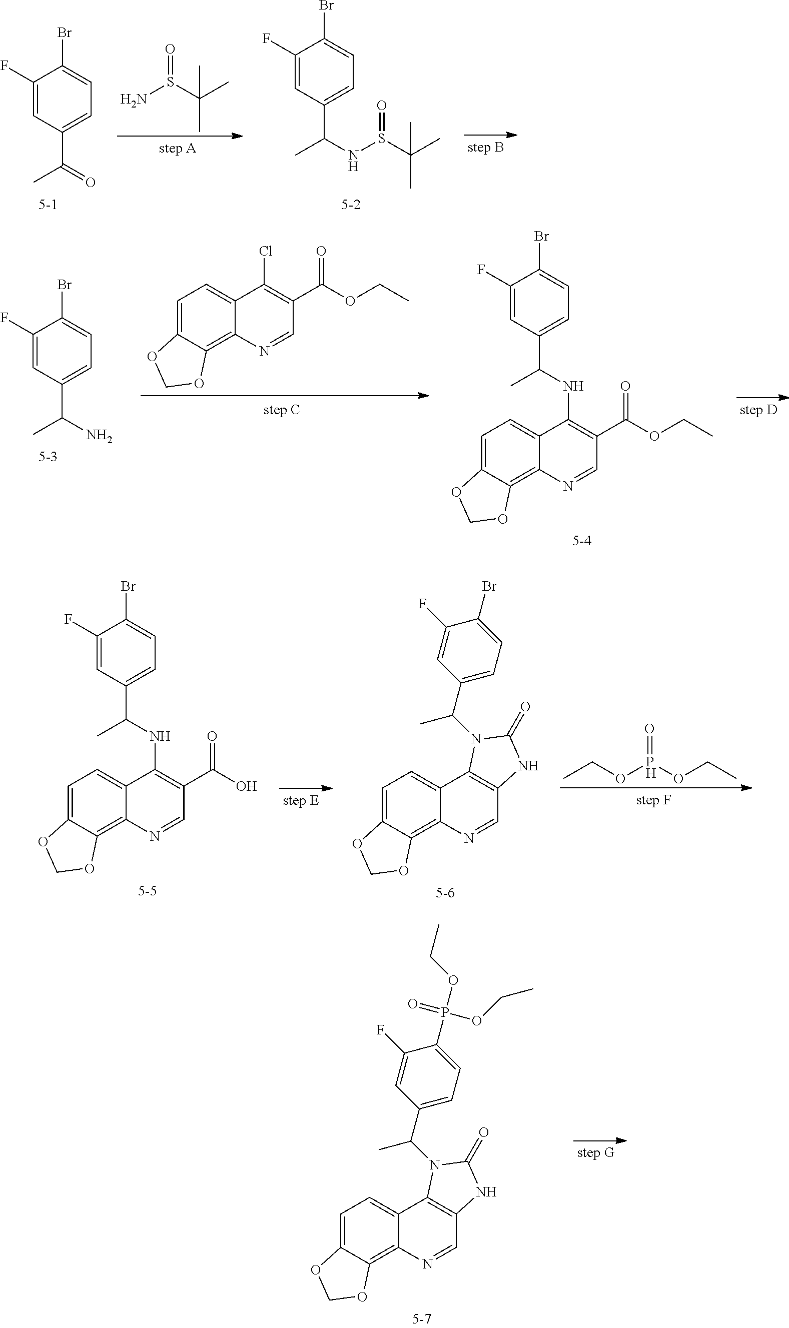

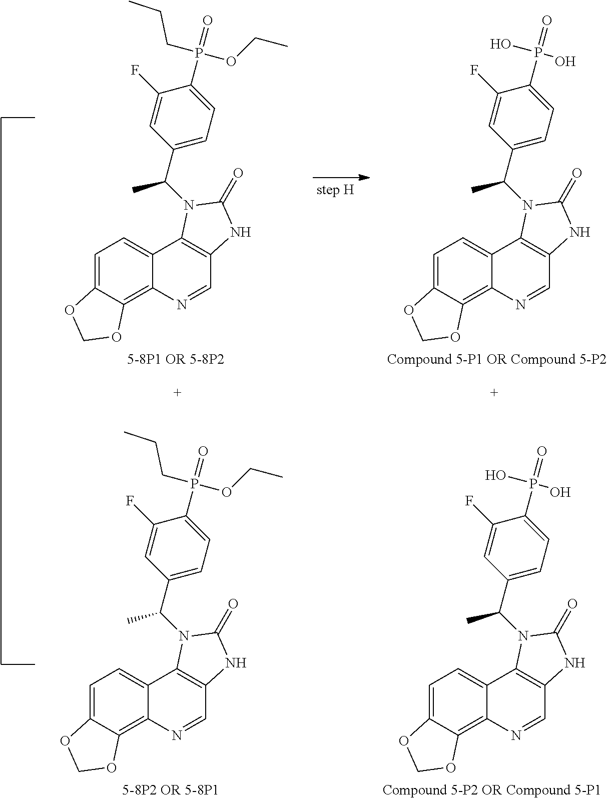 [SMILES: CCCP(=O)(OCC)c1ccc([C@H](C)n2c(=O)[nH]c3cnc4c5c(ccc4c32)OCO5)cc1F ; C[C@@H](c1ccc(P(=O)(O)O)c(F)c1)n1c(=O)[nH]c2cnc3c4c(ccc3c21)OCO4 ; CCCP(=O)(OCC)c1ccc([C@@H](C)n2c(=O)[nH]c3cnc4c5c(ccc4c32)OCO5)cc1F ; C[C@@H](c1ccc(P(=O)(O)O)c(F)c1)n1c(=O)[nH]c2cnc3c4c(ccc3c21)OCO4]

Step A: In a 100 mL dry reaction flask placed under nitrogen atmosphere, 1-(4-bromo-3-fluorophenyl)ethan-1-one (3.0 g, 13.82 mmol), 2-methylpropan-2-sulfinamide (2.0 g, 16.59 mmol) and tetraisopropyl titanate (7.86 g, 27.6 mmol) were dissolved in anhydrous tetrahydrofuran (30 mL) at room temperature, and the solution was stirred at 80° C. for 15 h. The reaction was monitored using LCMS and TLC, and when the reaction was finished, the reaction solution was cooled to room temperature and filtered. The filtrate was concentrated and dried to give a crude product. The crude product was dissolved in tetrahydrofuran (30 mL), lithium borohydride was added dropwise to the solution at 0° C., and the reaction was then slowly heated to room temperature and continued for 3 h. The reaction was monitored by LCMS and TLC, and when the reaction was finished, the reaction solution was filtered through sodium sulfate decahydrate, and the filtrate was concentrated and dried in vacuum to give 742.0 mg of Intermediate 5-2.

MS (ESI) M/Z: 322.2 [M+H]⁺.

Step B: In a 50 mL dry single-necked flask, N-(1-(4-bromo-3-fluorophenyl)ethyl)-2-methylpropan-2-sulfinamide (742.0 mg, 2.31 mmol) was dissolved in anhydrous methanol (12 mL) at room temperature, followed by the addition of hydrogen chloride solution in 1,4-dioxane (4 moles per liter, 1122 μL), and the reaction was stirred for 18 h at room temperature. The reaction was monitored using LCMS and TLC, and when the reaction was finished, the reaction solution was concentrated under reduced pressure. Water (10 mL) was added to the reaction solution, and the solution was extracted with dichloromethane (30 mL). The combined organic layer was washed three times with saturated saline (10 mL), dried over anhydrous sodium sulfate, filtered, and concentrated to obtain a crude product, which was purified by silica gel column chromatography to give 330.0 mg of Intermediate 5-3.

Step C: In a 50 mL dry three-necked flask placed in a nitrogen atmosphere, Intermediate 5-3 (200.0 mg, 0.72 mmol) and 1-(4-bromo-3-fluorophenyl)ethan-1-amine (170.6 mg, 0.786 mmol) were dissolved in anhydrous acetonitrile (6.0 mL), followed by the addition of triethylamine (0.21 mL, 1.584 mmol), and the solution was stirred for 15 h at 100° C. The reaction was monitored using LCMS and TLC, and when the reaction was finished, the reaction solution was cooled to room temperature, petroleum ether/ethyl acetate (V/V=1/1, 26 mL) was added to the reaction system, and the mixture was stirred for 1 h and filtered. The filter cake was washed three times with petroleum ether/ethyl acetate (V/V=1/1, 20 mL) dried in vacuum to give 238.0 mg of Intermediate 5-4.

MS (ESI) M/Z: 461.8 [M+H]⁺.

Step D: In a 50 mL dry three-necked flask, Intermediate 5-4 (238.0 mg, 0.52 mmol) was dissolved in a mixture of anhydrous tetrahydrofuran/water/ethanol (V/V/V=1/1/1, 7.5 mL) at room temperature, followed by the addition of sodium hydroxide (171.8 mg, 4.3 mmol), and the solution was stirred for 6 h at 60° C. The reaction was monitored using LCMS and TLC, and when the reaction was finished and cooled to room temperature, the reaction solution was concentrated under reduced pressure. Water (20 mL) was added to the resulting residue, and dilute hydrochloric acid (2 mol/L) was added dropwise to adjust the pH to 5. The precipitated solid was filtered and then washed twice with water (10 mL) and dried to give 138.0 mg of Intermediate 5-5.

MS (ESI) M/Z: 433.8 [M+H]⁺.

Step E: In a 50 mL dry single-necked flask. Intermediate 5-5 (138.0 mg, 0.32 mmol) was dissolved in toluene (2 mL) at room temperature, diphenyl azidophosphate (175.8 mg, 0.64 mmol) and triethylamine (129.3 mg, 1.278 mmol) were added dropwise, and the reaction was stirred for 15 h at 120° C. The reaction was monitored using LCMS and TLC, and when the reaction was finished and cooled to room temperature, the reaction solution was concentrated under reduced pressure, and the resulting residue was separated and purified by silica gel column chromatography to give 59.2 mg of Intermediate 5-6.

MS (ESI) M/Z: 430.8 [M+H]⁺.

Step F: In a 10 mL dry microwave tube placed under nitrogen atmosphere, Intermediate 5-6 (59.2 mg, 0.1 mmol) was dissolved in N,N-dimethylformamide (3 mL) at room temperature, followed by the addition of diethyl phosphite (57.2 mg, 0.41 mmol), [1,1′-bis(diphenylphosphino)ferrocene]palladium(II) dichloride dichloromethane complex (11.3 mg, 0.02 mmol) and triethylamine (55.0 mg, 0.55 mmol), and the solution was stirred for 3 h at 100° C. under microwave. The reaction was monitored using LCMS and TLC, and when the reaction was finished and cooled to room temperature, water (20 mL) was added to the reaction solution, and the solution was extracted with dichloromethane (50 mL). The combined organic layer was washed three times with saturated saline (20 mL), dried over anhydrous sodium sulfate, filtered, and concentrated to obtain a crude product, which was purified by silica gel column chromatography to give 36.0 mg of Intermediate 5-7.

MS (ESI) M/Z: 488.0 [M+H]⁺.

Step G: Diethyl (2-fluoro-4-(1-(7-oxo-6,7-dihydro-8H-[1,3]dioxolo[4,5-h]imidazo[4,5-c]quinolin-8-yl)ethyl)phenyl)phosphonate (36 mg) was subjected to chiral resolution (mobile phase: MeOH [0.2% NH₃ (7 M in MeOH)], chromatographic column: AD-34.6*100 mm 3 μm) to obtain 13.0 mg of Intermediate 5-8P1 (retention time 0.982 min) and 13.3 mg of Intermediate 5-8P2 (retention time 1.428 min), 5-8P1 and 5-8P2 are enantiomers of each other, and the absolute configurations of which were not determined.

Step H: In a 50 mL dry single-necked flask, Intermediate 5-8P1 (13.3 mg, 0.0273 mmol) and Intermediate 5-8P2 (13.3 mg, 0.0273 mmol) obtained by separation in step G were dissolved in anhydrous acetonitrile (1.5 mL) at room temperature, followed by the dropwise addition of trimethyl bromosilane (1.5 mL), and the reaction was stirred for 20 h at room temperature. The reaction was monitored using LCMS and TLC, and when the reaction was completed, the reaction solution was concentrated and the residue was purified by preparative high performance liquid chromatography to give 6.6 mg of Compound 5-P1 and 3.3 mg of Compound 5-P2.

### Compound 5-P1:

MS (ESI) M/Z: 431.9 [M+H]⁺.

¹H NMR (400 MHz, DMSO-d6) δ 8.66 (s, 1H), 7.65-7.57 (m, 1H), 7.25-6.95 (m, 4H), 6.35-6.06 (m, 3H), 1.91 (d, J=6.5 Hz, 3H).

### Compound 5-P2:

MS (ESI) M/Z: 431.8 [M+H].

¹H NMR (400 MHz, DMSO-d6) δ 8.65 (s, 1H), 7.76-7.53 (m, 1H), 7.31-6.91 (m, 4H), 6.48-5.94 (m, 3H), 1.91 td, J=7.7 Hz, 3H).

### Example 6: (4-((6-ethyl-7-oxo-6,7-dihydro-8H-[1,3]dioxolo[4,5-h]imidazo[4,5-c]quinolin-8-yl)methyl)-2-fluorophenyl)phosphonic acid

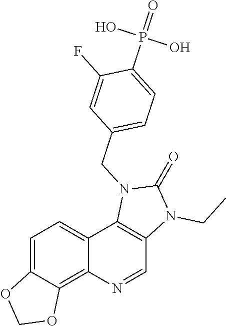 [SMILES: CCn1c(=O)n(Cc2ccc(P(=O)(O)O)c(F)c2)c2c3ccc4c(c3ncc21)OCO4]

Referring to Example 3, from Intermediate 1-8 (10 mg, 0.2 mmol) and ethyl iodide, 11.71 mg of Compound 6 was prepared.

MS (ESI) M/Z: 445.8 [M+H].

¹H NMR (400 MHz, DMSO-d6) δ 8.95 (s, 1H), 7.65-7.53 (m, 1H), 7.48 (d, J=9.0 Hz, 1H), 7.30 (d, J=9.0 Hz, 1H), 7.15-7.06 (m, 1H), 7.03 (d, J=7.9 Hz, 1H), 6.21 (s, 2H), 5.57 (s, 2H), 4.13 (q, J=7.1 Hz, 2H), 1.35 (t, J=7.1 Hz, 3H).

The following target compounds were prepared with reference to the synthesis method of Example 1 above:

| | | | MS (ESD) |
|---|---|---|---|
| Compound | | | M/Z |
| No | Structural formula | ¹HNMR | [M + H]⁺ |
| 7 | 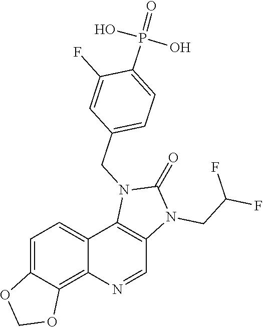 [SMILES: O=c1n(CC(F)F)c2cnc3c4c(ccc3c2n1Cc1ccc(P(=O)(O)O)c(F)c1)OCO4] | (400 MHz, CD₃OD) δ 8.83 (s, 1H), 7.76 (s, 1H), 7.58 (d, J = 9.0 Hz, 1H), 7.22 (d, J = 9.0 Hz, 1H), 7.13-6.96 (m, 2H), 6.61- 6.31 (m, 1H), 6.22 (s, 2H), 5.66 (s, 2H), 4.71-4.48 (m, 2H). | 482.0 |
| 8 | 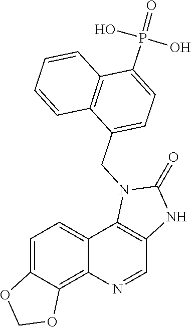 [SMILES: O=c1[nH]c2cnc3c4c(ccc3c2n1Cc1ccc(P(=O)(O)O)c2ccccc12)OCO4] | (400 MHz, DMSO-d6) δ 8.77 (d, J = 8.6 Hz, 1H), 8.73 (s, 1H), 8.31 (d, J = 8.5 Hz, 1H), 7.75 (q, J = 8.0 Hz, 1H), 7.64 (t, J = 7.7 Hz, 1H), 7.51 (t, J = 7.8 Hz, 1H), 7.16 (d, J = 9.0 Hz, 1H), 7.09 (d, J = 8.9 Hz, 1H), 6.79 (d, 1H), 6.18 (s, 2H), 5.96 (s, 2H). | 449.8 |
| 9 | 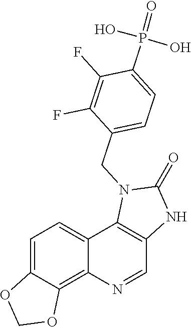 [SMILES: O=c1[nH]c2cnc3c4c(ccc3c2n1Cc1ccc(P(=O)(O)O)c(F)c1F)OCO4] | (400 MHz, DMSO-d6) δ 11.74 (s, 1H), 8.67 (s, 1H), 7.86-7.23 (m, 3H), 6.75 (s, 1H), 6.20 (s, 2H), 5.57 (s, 2H). | 435.8 |
| 10 | 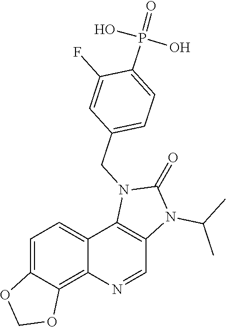 [SMILES: CC(C)n1c(=O)n(Cc2ccc(P(=O)(O)O)c(F)c2)c2c3ccc4c(c3ncc21)OCO4] | (400 MHz, CD₃OD) δ 8.09 (s, 1H), 7.05-6.89 (m, 1H), 6.77 (d, J = 9.0 Hz, 1H), 6.38 (d, J = 9.0 Hz, 1H), 6.22 (d, J = 7.7 Hz, 1H), 6.17-6.10 (m, 1H), 5.40 (s, 2H), 4.82 (s, 2H), 4.22-4.05 (m, 1H), 0.88 (d, J = 6.9 Hz, 6H). | 460.0 |
| 11 | 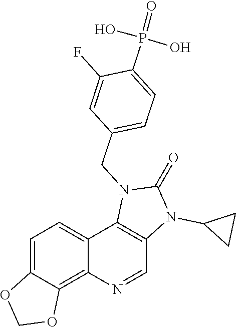 [SMILES: O=c1n(Cc2ccc(P(=O)(O)O)c(F)c2)c2c3ccc4c(c3ncc2n1C1CC1)OCO4] | (400 MHz, DMSO-d6) δ 8.89 (s, 1H), 7.63-7.51 (m, 1H), 7.46 (d, J = 9.0 Hz, 1H), 7.28 (d, J = 8.9 Hz, 1H), 7.05-6.93 (m, 2H), 6.19 (s, 2H), 5.48 (s, 2H), 3.20-3.11 (m, 1H), 1.19-1.08 (m, 2H), 1.11-1.01 (m, 2H). | 458.2 |
| 12 | 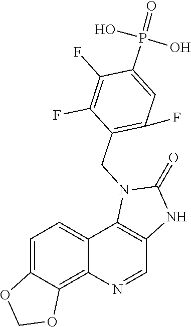 [SMILES: O=c1[nH]c2cnc3c4c(ccc3c2n1Cc1c(F)cc(P(=O)(O)O)c(F)c1F)OCO4] | (400 MHz, DMSO-d6) δ 8.61 (s, 1H), 7.67 (d, J = 8.9 Hz, 1H), 7.34 (d, J = 8.9 Hz 1H), 7.21- 7.03 (m, 1H), 6.19 (s, 2H), 5.58 (s, 2H). | 454.0 |
| 13 | 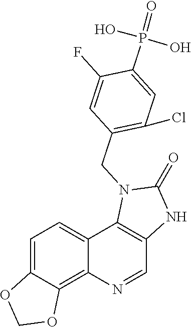 [SMILES: O=c1[nH]c2cnc3c4c(ccc3c2n1Cc1cc(F)c(P(=O)(O)O)cc1Cl)OCO4] | (400 MHz, DMSO-d6) δ 8.68 (s, 1H), 7.69 (dd, J = 12.8, 5.6 Hz, 1H), 7.27 (d, J = 8.9 Hz, 1H), 7.18 (d, J = 8.9 Hz, 1H), 6.65- 6.44 (m, 1H), 6.19 (s, 2H), 5.45 (s, 2H). | 451.9 |
| 14 | 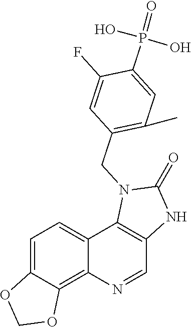 [SMILES: Cc1cc(P(=O)(O)O)c(F)cc1Cn1c(=O)[nH]c2cnc3c4c(ccc3c21)OCO4] | (400 MHz, DMSO-d6) δ 11.78 (s, 1H), 871 (s, 1H), 7.59 (m, 1H), 7.28 (d, J = 8.9 Hz, 1H), 7.21 (d, J = 8.9 Hz, 1H), 6.39 (dd, J = 10.2, 5.5 Hz, 1H), 6.22 (s, 2H), 5.45 (s, 2H), 2.48 (s, 3H). | 432.0 |
| 15 | 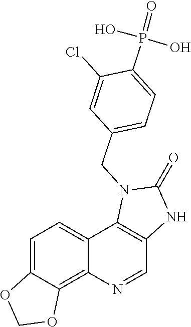 [SMILES: O=c1[nH]c2cnc3c4c(ccc3c2n1Cc1ccc(P(=O)(O)O)c(Cl)c1)OCO4] | (400 MHz, DMSO-d6) δ 11.72 (s, 1H), 7.76 (dd, J = 13.4, 7.9 Hz, 1H), 7.48 (d, J = 8.9 Hz, 1H), 7.40 (d, J = 4.4 Hz, 1H), 7.30 (d, J = 8.9 Hz, 1H), 7.19 (d, J = 8.1 Hz, 1H), 6.20 (s, 2H), 5.54 (s, 2H). | 433.9 |
| 16 | 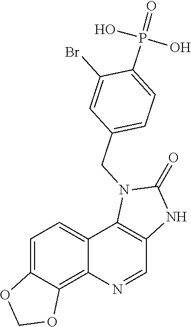 [SMILES: O=c1[nH]c2cnc3c4c(ccc3c2n1Cc1ccc(P(=O)(O)O)c(Br)c1)OCO4] | (400 MHz, DMSO-d6) δ 11.75 (s, 1H), 8.68 (s, 1H), 7.82-7.72 (m, 1H), 7.62-7.57 (m, 1H), 7.50 (d, J = 9.0 Hz, 1H), 7.31 (d, J = 8.9 Hz, 1H), 7.27-7.19 (m, 1H), 6.21 (s, 2H), 5.54 (s, 2H). | 479.1 |
| 17 | 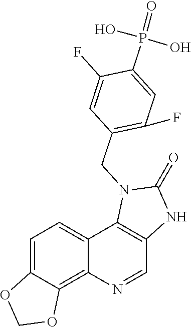 [SMILES: O=c1[nH]c2cnc3c4c(ccc3c2n1Cc1cc(F)c(P(=O)(O)O)cc1F)OCO4] | (400 MHz, DMSO-d6) δ 8.67 (s, 1H), 743 (d, J = 9.0 Hz, 1H), 7.40-7.33 (m, 1H), 7.31 (d, J = 8.9 Hz, 1H), 6.56-6.46 (m, 1H), 6.20 (s, 2H), 5.47 (s, 2H). | 436.0 |
| 18 | 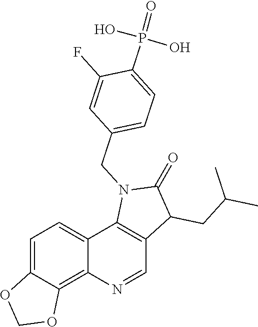 [SMILES: CC(C)CC1C(=O)N(Cc2ccc(P(=O)(O)O)c(F)c2)c2c1cnc1c3c(ccc21)OCO3] | (400 MHz, DMSO-d6) δ 8.94 (s, 1H), 7.66-7.43 (m, 2H), 7.29 (d, J = 9.0 Hz, 1H), 7.00-6.77 (m 2H), 6.19 (s, 2H), 5.52 (s, 2H), 3.91 (d, J = 7.4 Hz, 2H), 2.29-2.17 (m, 1H), 0.95 (d, J = 6.6 Hz, 6H). | 474.0 |
| 19 | 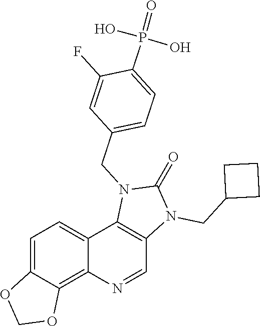 [SMILES: O=c1n(CC2CCC2)c2cnc3c4c(ccc3c2n1Cc1ccc(P(=O)(O)O)c(F)c1)OCO4] | (400 MHz, DMSO-d6) δ 8.94 (s, 1H), 7.58-7.45 m, 2H), 7.29 (d, J = 9.0 Hz, 1H), 6.90 (d, J = 8.2 Hz, 2H), 6.19 (s, 2H), 5.53 (s, 2H), 4.13 (d, J = 7.2 Hz, 2H), 2.96-2.78 (m, 1H), 2.08-1.94 (m, 2H), 1.92-1.80 (m, 4H). | 486.2 |
| 20 | 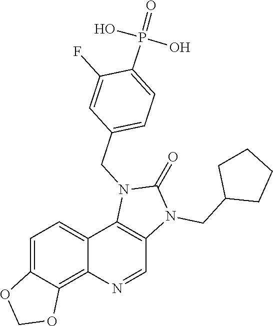 [SMILES: O=c1n(CC2CCCC2)c2cnc3c4c(ccc3c2n1Cc1ccc(P(=O)(O)O)c(F)c1)OCO4] | (400 MHz, DMSO-d6) δ 8.95 (s, 1H), 7.58-7:45 (m, 2H), 7.29 (d, J = 9.0 Hz, 1H), 6.94-6.88 (m, 2H), 6.19 (s, 2H), 5.53 (s, 2H), 4.02 (d, J = 7.6 Hz, 2H), 2.49-2.43 (m, 1H), 1.74-1.61 (m, 4H), 1.57-1.47 (m, 2H), 1.41-1.28 (m, 2H). | 500.0 |
| 21 | 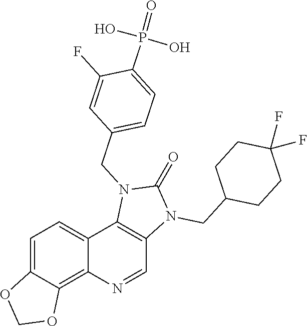 [SMILES: O=c1n(CC2CCC(F)(F)CC2)c2cnc3c4c(ccc3c2n1Cc1ccc(P(=O)(O)O)c(F)c1)OCO4] | (400 MHz, DMSO-d6) δ 8.98 (s, 1H), 7.59-7.51 (m,1H), 7.48 (d, J = 9.1 Hz, 1H), 7.30 (d, J = 9.0 Hz, 1H), 6.95-6.89 (m, 2H). 6.19 (s, 2H), 5.52. (s, 2H), 4.01 (d, J = 7.3 Hz, 2H), 2.19- 1.96 (m, 3H), 1.92-1.64 (m, 4H), 1.41-1.34 (m, 2H). | 550.1 |
| 22 | 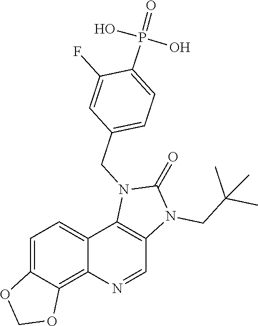 [SMILES: CC(C)(C)Cn1c(=O)n(Cc2ccc(P(=O)(O)O)c(F)c2)c2c3ccc4c(c3ncc21)OCO4] | (400 MHz, DMSO-d6) δ 8.95 (s, 1H), 7.67-7.45 (m, 2H), 7.29 (d, J = 9.0 Hz, 1H), 6.95-6.77 (m, 2H), 6.19 (s, 2H), 5.52 (s, 2H), 3.91 (s, 2H), 3.03 (s, 9H). | 488.2 |
| 23 | 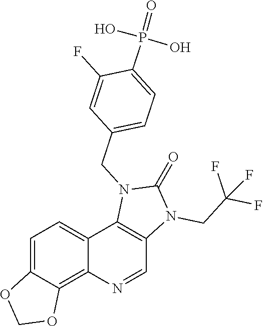 [SMILES: O=c1n(CC(F)(F)F)c2cnc3c4c(ccc3c2n1Cc1ccc(P(=O)(O)O)c(F)c1)OCO4] | (400 MHz, DMSO-d6) δ 9.00 (s, 1H), 7.60-7.50 (m, 2H), 7.33 (d, J = 9.0 Hz, 1H), 6.93-6.82 (m, 2H), 6.21 (s, 2H), 5.54 (s, 2H), 5.08 (q, J = 9.1 Hz, 2H). | 500.1 |
| 24 | 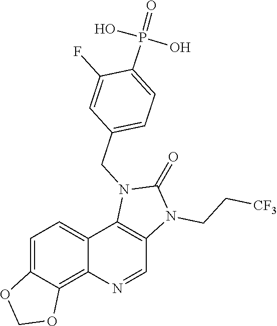 [SMILES: O=c1n(CCC(F)(F)F)c2cnc3c4c(ccc3c2n1Cc1ccc(P(=O)(O)O)c(F)c1)OCO4] | (400 MHz, DMSO-d6) δ 8.97 (s, 1H), 7.63-7.51 (m, 1H), 7.49 (d, J = 9.1 Hz, 1H), 7.30 (d, J = 8.9 Hz, 1H), 6.98-6.87 (m, 2H), 6.20 (s, 2H), 5.54 (s, 2H), 4.38 (t, J = 6.6 Hz, 2H), 2.97 2.88 (m, 2H). | 514.0 |
| 25 | 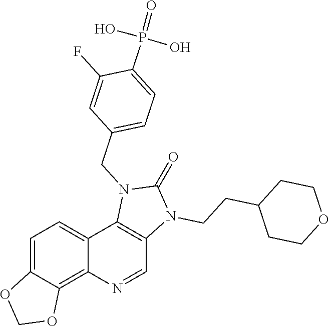 [SMILES: O=c1n(CCC2CCOCC2)c2cnc3c4c(ccc3c2n1Cc1ccc(P(=O)(O)O)c(F)c1)OCO4] | (400 MHz, Methanol-d4) δ 8.82 (s, 1H), 7.86-7.70 (m, 1H), 7.60 (d, J = 9.0 Hz, 1H), 7.23 (d, J = 9.0 Hz, 1H), 7.08 (d, J = 7.8 Hz, 1H), 7.04-6.95 (m, 1H), 6.23 (s, 2H), 5.66 (s, 2H), 4.26- 4.20 (m, 2H), 3.97-3.90 (m, 2H), 3.45-3.35 (m, 2H), 1.90- 1.74 (m, 4H), 1.45-1.28 (m, 3H). | 530.1 |
| 26 | 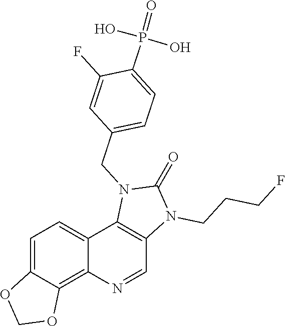 [SMILES: O=c1n(CCCF)c2cnc3c4c(ccc3c2n1Cc1ccc(P(=O)(O)O)c(F)c1)OCO4] | (400 MHz, DMSO-d6) δ 8.92 (s, 1H), 7.65-7.49 (m, 1H), 7.47 (d, J = 9.0 Hz. 1H), 7.29 (d, J = 9.0 Hz, 1H), 7.04-6.87 (m, 2H), 6.20 (s, 2H), 5.51 (s, 2H), 4.62 (t, J = 5.7 Hz, 1H), 4.0 (t, J = 5.6 Hz, 1H), 4.21 (t, J = 6.8 Hz, 2H), 2.29-2.18 (m, 1H), 2.17-2.09 (m, 1H). | 477.9 |
| 27 | 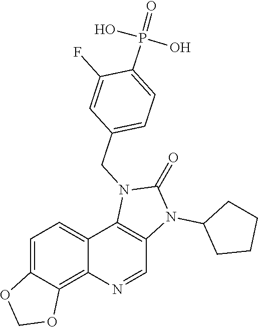 [SMILES: O=c1n(Cc2ccc(P(=O)(O)O)c(F)c2)c2c3ccc4c(c3ncc2n1C1CCCC1)OCO4] | (400 MHz, DMSO-d6) δ 8.94 (s, 1H), 7.63-7.55 (m, 1H), 7.47 (d, J = 9.0 Hz, 1H), 7.28 (d, J = 8.9 Hz, 1H), 7.10-6.85 (m, 2H), 6.20 (s, 2H), 5.52 (s, 2H), 4.97 (p, J = 8.6 Hz, 1H), 2.27- 2.13 (m, 2H), 2.12-2.01 (m, 2H), 1.99-1.94 (m, 2H), 1.73- 1.67 (m, 2H). | 486.2 |
| 28 | 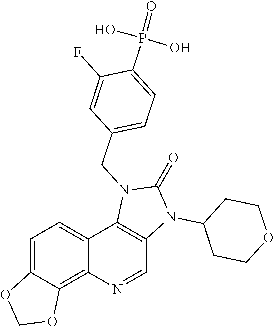 [SMILES: O=c1n(Cc2ccc(P(=O)(O)O)c(F)c2)c2c3ccc4c(c3ncc2n1C1CCOCC1)OCO4] | (400 MHz, DMSO-d6) δ 9.06 (s, 1H) 7.64-7.56 (m, 1H), 7.49 (d, J = 8.9 Hz, 1H), 7.30 (d, J = 9.0 Hz, 1H), 7.16-6.98 (m, 2H), 6.21 (s, 2H), 5.57 (s, 2H), 4.81-4.62 (m, 1H), 4.07-4.01 (m, 2H), 3.54 (t, J = 11.7 Hz, 4H), 1.89-1.81 (m, 2H). | 501.8 |
| 29 | 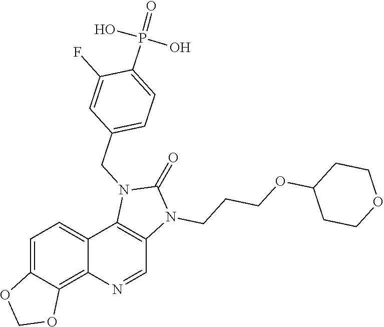 [SMILES: O=c1n(CCCOC2CCOCC2)c2cnc3c4c(ccc3c2n1Cc1ccc(P(=O)(O)O)c(F)c1)OCO4] | (400 MHz, DMSO-d6) δ 8.92 (s, 1H), 7.66-7.54 (m, 1H), 7.49 8.9 Hz, 1H), 7.14-7.02 (m, 2H), 6.21 (s, 2H), 5.58 (s, 2H), 4.16 (t, J = 6.7 Hz, 2H), 3.78- 3.70 (m, 2H), 3.52-3.45 (m, 2H), 3.44-3.35 (m, 1H), 3.32- 3.25 (m, 2H), 2.06-1.99 (m, 2H), 1.78-1.73 (m, 2H), 1.39- 1.25 (m, 2H). | 560.0 |
| 30 | 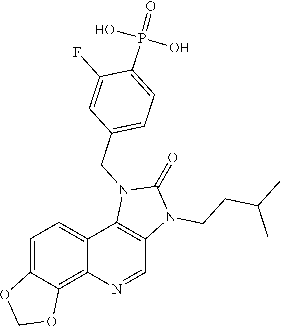 [SMILES: CC(C)CCn1c(=O)n(Cc2ccc(P(=O)(O)O)c(F)c2)c2c3ccc4c(c3ncc21)OCO4] | (400 MHz, DMSO-d6) δ 8.93 (s, 1H), 7.66-7.54 (m, 1H), 7.50 (d, J = 9.0 Hz, 1H), 7.31 (d, J = 8.9 Hz, 1H), 7.17-6.93 (m, 2H), 6.21 (s, 2H), 5.58 (s, 2H), 4.11 (t, J = 7.1 Hz, 2H), 1.74- 1.56 (m, 3H), 0.96 (d, J = 6.4 Hz, 6H). | 489.2 |
| 31 | 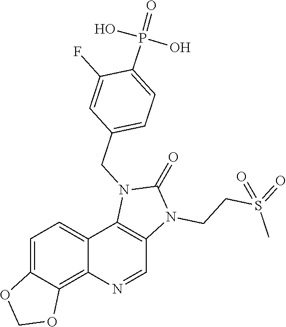 [SMILES: CS(=O)(=O)CCn1c(=O)n(Cc2ccc(P(=O)(O)O)c(F)c2)c2c3ccc4c(c3ncc21)OCO4] | (400 MHz, DMSO-d6) δ 8.99 (s, 1H), 7.65-7.54 (m, 1H), 7.47 (d, J = 9.0 Hz, 1H), 7.30 (d, J = 9.0 Hz, 1H), 7.16-7.10 (m, 2H), 6.21 (s, 2H), 5.59 (s, 2H), 4.56 (t, J = 6.5 Hz, 2H), 3.74 (t, J = 6.5 Hz, 2H), 3.13 (s, 3H). | 523.7 |
| 32 | 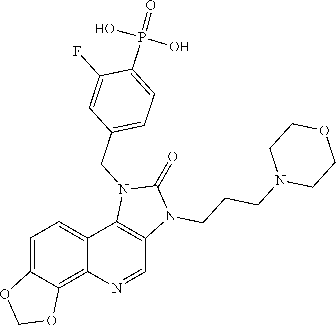 [SMILES: O=c1n(CCCN2CCOCC2)c2cnc3c4c(ccc3c2n1Cc1ccc(P(=O)(O)O)c(F)c1)OCO4] | (400 MHz, DMSO-d6) δ 9.32 (s, (1H, 7.82 (d, J = 9.2 Hz, 1H), 7.71-7.62 (m, 1H), 7.60 (d, J = 9.1 Hz, 1H), 7.27-7.14 (m, 2H), 6.46 (s, 2H), 5.74 (s, 2H), 4.24 (t, J = 6.6 Hz, 2H), 3.97 (d, J = 13.1 Hz, 2H), 3.64 (t, J = 12.1 Hz, 2H), 3.42 (d, J = 12.2 Hz, 2H), 3.30-3.19 (m, 2H), 3.13-3.01 (m 2H), 2.28- 2.13 (m, 2H). | 545.1 |
| 33 | 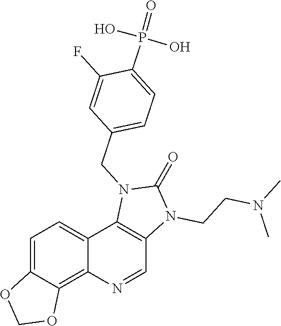 [SMILES: CN(C)CCn1c(=O)n(Cc2ccc(P(=O)(O)O)c(F)c2)c2c3ccc4c(c3ncc21)OCO4] | (400 MHz, DMSO-d6) δ 8.95 (s, 1H), 7.77-7.61 (m, 1H), 7.53 (d, J = 8.9 Hz, 1H), 7.28 (d, J = 9.1 Hz, 1H), 6.96 (d, J = 8.1 Hz, 1H), 6.91-6.78 (m, 1H), 6.20 (s, 2H), 5.54 (s, 2H), 4.20 (t, J = 6.0 Hz, 2H), 2.81-2.69 (m, 2H), 2.23 (s, 6H). | 488.8 |
| 34 | 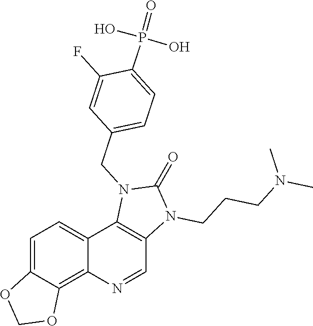 [SMILES: CN(C)CCCn1c(=O)n(Cc2ccc(P(=O)(O)O)c(F)c2)c2c3ccc4c(c3ncc21)OCO4] | (400 MHz, DMSO-d6) δ 9.30 (s, 1H), 7.81 (d, J = 9.2 Hz, 1H), 7.72-7.62 (m, 1H), 7.60 (d, J = 9.1 Hz, 1H), 7.28-7.15 (m, 2H), 6.46 (s, 2H), 5.74 (s, 2H), 4.22 (t, J = 6.7 Hz, 2H), 3.29- 3.12 (m, 2H), 2.79 (s, 6H), 2.32- 2.06 (m, 2H). | 503.1 |
| 49 | 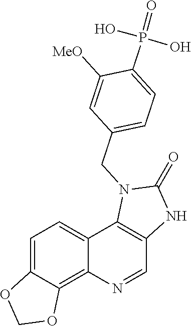 [SMILES: COc1cc(Cn2c(=O)[nH]c3cnc4c5c(ccc4c32)OCO5)ccc1P(=O)(O)O] | (400 MHz, DMSO-d6) δ 8.87 (s, 1H), 7.85 (d, J = 9.1 Hz, 1H), 7.58 (d, J = 9.2 Hz, 1H), 7.53 (dd, J = 14.6, 7.8 Hz, 1H), 7.17 (d, J = 5.7 Hz, 1H), 6.73 (d, J = 7.3 Hz, 1H), 6.44 (s, 2H), 5.65 (s, 2H), 3.79 (s, 3H). | 430.0 |
| 50 | 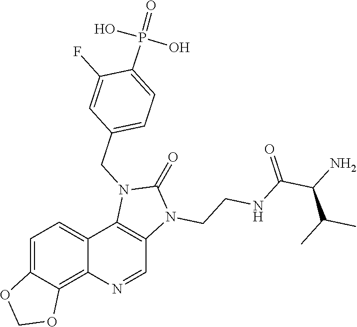 [SMILES: CC(C)[C@H](N)C(=O)NCCn1c(=O)n(Cc2ccc(P(=O)(O)O)c(F)c2)c2c3ccc4c(c3ncc21)OCO4] | (400 MHz, DMSO-d6) δ 8.99 (s, 1H), 8.81 (s, 1H), 7.74-7.65 (m, 1H), 7.49 (d, J = 9.0 Hz, 1H), 7.01-6.91 (m, 2H), 6.21 (s, 2H), 5.51 (s, 2H), 4.17 (s, 1H), 3.81-3.73 (m, 1H), 2.00 1.91 (m, 1H), 0.78 (d, J = 6.8 Hz, 3H), 0.69 (d, J = 6.8 Hz, 3H). | 559.6 |
| 51 | 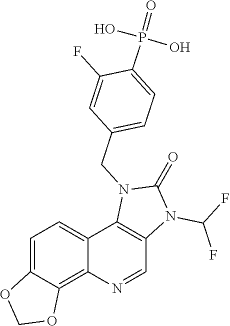 [SMILES: O=c1n(Cc2ccc(P(=O)(O)O)c(F)c2)c2c3ccc4c(c3ncc2n1C(F)F)OCO4] | (400 MHz, DMSO-d6) δ 8.93 (s, 1H), 8.09-7.76 (m, 1H), 7.76- 7.64 (m, 1H), 7.53 (d, J = 9.0 Hz, 1H), 7.35 (d, J = 8.9 Hz, 1H), 7.09-6.99 (m, 2H), 6.25 (s, 2H), 5.54 (s, 2H). | 467.8 |
| 52 | 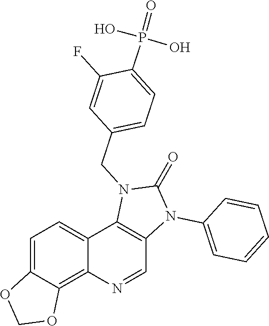 [SMILES: O=c1n(Cc2ccc(P(=O)(O)O)c(F)c2)c2c3ccc4c(c3ncc2n1-c1ccccc1)OCO4] | (400 MHz, DMSO-d6) δ 8.63 (s, 1H), 7.76-7.70 (m, 2H), 7.68 7.60 (m, 3H), 7.56-7.50 (m, 2H), 7.37-7.27 (m, 1H), 7.17- 6.94 (m, 2H), 6.20 (s, 2H), 5.58 (s, 2H). | 493.8 |
| 55 | 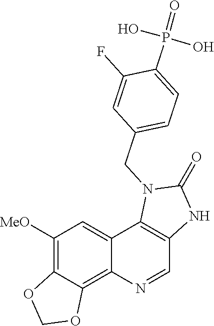 [SMILES: COc1cc2c(ncc3[nH]c(=O)n(Cc4ccc(P(=O)(O)O)c(F)c4)c32)c2c1OCO2] | (400 MHz, DMSO-d6) δ 8.53 (s, 1H), 7.43-7.25 (m, 2H), 7.01- 6.86 (m, 1H), 6.74 (s, 1H), 6.16 (s, 2H), 5.53 (s, 2H), 3.64 (s, 3H). | 448.0 |
| 56 | 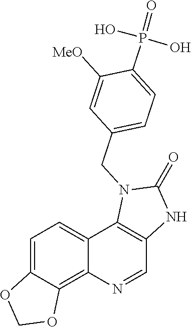 [SMILES: COc1cc(Cn2c(=O)[nH]c3cnc4c5c(ccc4c32)OCO5)ccc1P(=O)(O)O] | (400 MHz, DMSO-d6) δ 8.87 (s, 1H), 7.85 (d, J = 9.1 Hz, 1H), 7.58 (d, J = 9.2 Hz, 1H), 7.53 (dd, J = 14.6, 7.8 Hz, 1H), 7.17 (d, J = 5.7 Hz, 1H), 6.73 (d, J = 7.3 Hz, 1H), 6.44 (s, 2H), 5.65 (s, 2H), 3.79 (s, 3H). | 430.0 |
| 57 | 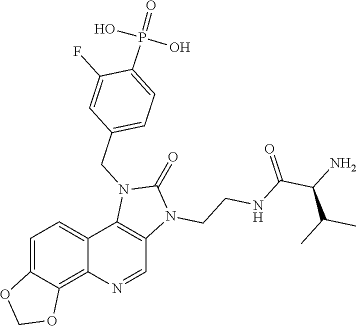 [SMILES: CC(C)[C@H](N)C(=O)NCCn1c(=O)n(Cc2ccc(P(=O)(O)O)c(F)c2)c2c3ccc4c(c3ncc21)OCO4] | (400 MHz, DMSO-d6) δ 8.99 (s, 1H), 8.81 (s, 1H), 7.74-7.65 (m, 1H), 7.49 (d, J = 9.0 Hz, 1H), 7.01-6.91 (m, 2H), 6.21 (s, 21), 5.51 (s, 2H), 4.17 (s, 1H), 3.81-3.73 (m, 1H), 2.00 1.91 (m, 1H), 0.78 (d, J = 6.8 Hz, 3H), 0.69 (d, J = 6.8 Hz, 3H). | 559.6 |
| 58 | 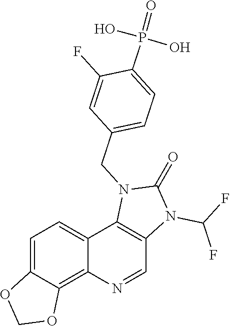 [SMILES: O=c1n(Cc2ccc(P(=O)(O)O)c(F)c2)c2c3ccc4c(c3ncc2n1C(F)F)OCO4] | (400 MHz, DMSO-d6) δ 8.93 (s, 1H), 8.09-7.76 (m, 1H), 7.76- 7.64 (m, 1H), 7.53 (d, J = 9.0 Hz, 1H), 7.35 (d, J = 8.9 Hz, 1H), 7.09-6.99 (m, 2H), 6.25 (s, 2H), 5.54 (s, 2H). | 467.8 |
| 59 | 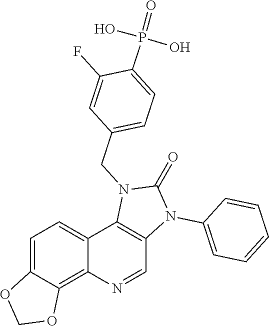 [SMILES: O=c1n(Cc2ccc(P(=O)(O)O)c(F)c2)c2c3ccc4c(c3ncc2n1-c1ccccc1)OCO4] | (400 MHz, DMSO-d6) δ 8.63 (s, 1H), 7.76-7.70 (m, 2H), 7.68- 7.60 (m, 3H), 7.56-7.50 (m, 2H), 7.37-7.27 (m, 1H), 7.17- 6.94 (m, 2H), 6.20 (s, 2H), 5.58 (s, 2H). | 493.8 |
| 60 | 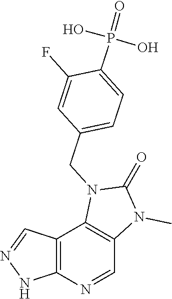 [SMILES: Cn1c(=O)n(Cc2ccc(P(=O)(O)O)c(F)c2)c2c3cn[nH]c3ncc21] | (400 MHz, DMSO-d6) δ 8.47 (s, 1H), 8.08 (s, 1H), 7.68-7.57 (m, 1H), 7.29-7.17 (m, 2H), 5.38 (s, 2H), 3.51 (s, 3H). | 378.0 |
| 61 | 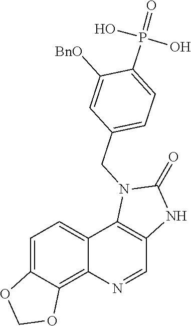 [SMILES: O=c1[nH]c2cnc3c4c(ccc3c2n1Cc1ccc(P(=O)(O)O)c(OCc2ccccc2)c1)OCO4] | (400 MHz, DMSO-d6) δ 8.82 (s, 1H), 7.67-7.52 (m, 3H), 7.37 (d, J = 9.1 Hz, 1H), 7.29-7.22 (m, 2H), 7.16-7.11 (m, 2H), 6.86 (d, J = 7.5 Hz, 1H), 6.79 (d, J = 5.6 Hz, 1H), 5.54 (s, 3H), 5.12 (s, 2H). | 506.0 |

### Example 7: 3,5-difluoro-4-(7-oxo-6,7-dihydro-8H-[1,3]dioxolo[4,5-h]imidazo[4,5-c]quinolin-S-yl)methyl)benzenesulfonamide

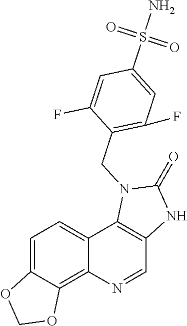 [SMILES: NS(=O)(=O)c1cc(F)c(Cn2c(=O)[nH]c3cnc4c5c(ccc4c32)OCO5)c(F)c1]

### Reaction Route:

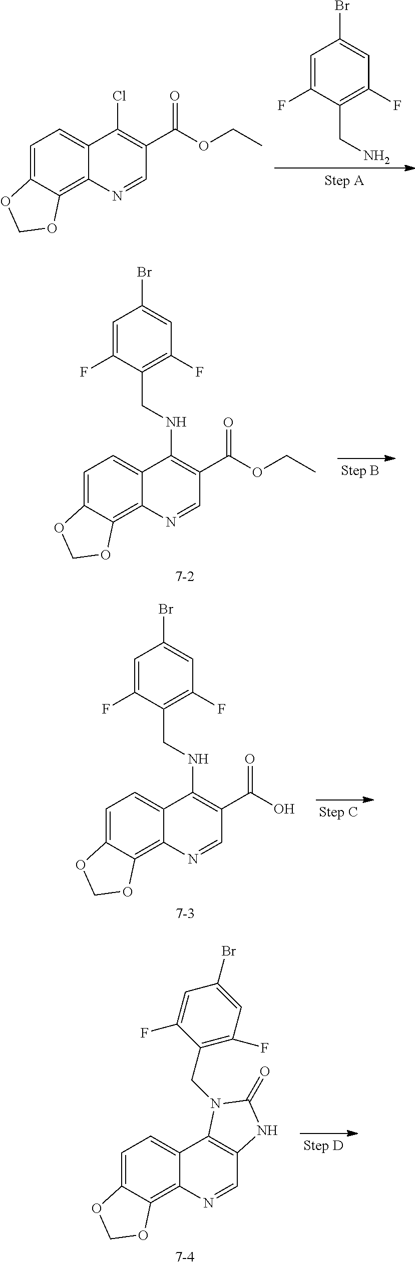

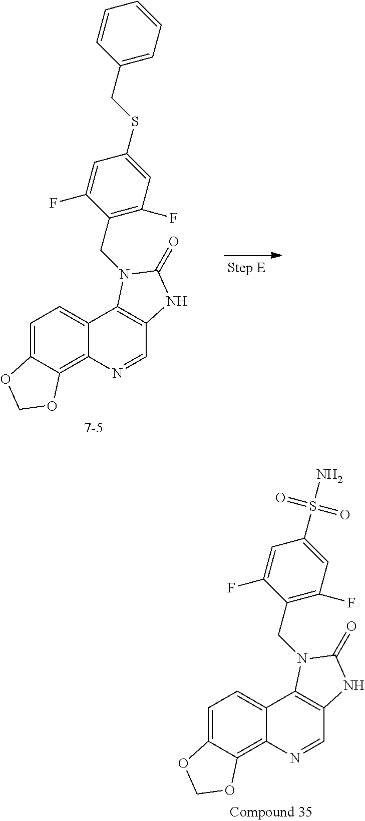 [SMILES: O=c1[nH]c2cnc3c4c(ccc3c2n1Cc1c(F)cc(SCc2ccccc2)cc1F)OCO4 ; NS(=O)(=O)c1cc(F)c(Cn2c(=O)[nH]c3cnc4c5c(ccc4c32)OCO5)c(F)c1]

### Operating Procedure:

Step A: Compound ethyl 6-chloro-[1,3]dioxolo[4,5-h]quinolin-7-formate (300 mg, 1.08 mmol) was dissolved in N,N-dimethylformamide (5 mL), and 4-bromo-2,6-difluorobenzenemethylamine (356 mg, 1.6 mmol), and N,N-diisopropylethylamine (557 mg, 4.32 mmol) were added to the solution in sequence. The reaction carried out at 100° C. for 2 h under microwave.

After the depletion of raw material as monitored by LCMS, the reaction solution was added with water. The resultant was extracted twice with ethyl acetate, the organic phases were combined, and the combined organic phase was dried with anhydrous sodium sulfate, filtered, and concentrated under reduced pressure. The residue was purified by silica gel column chromatography to obtain 480 mg of Intermediate 7-2.

MS (ESI) M/Z: 464.9 [M+H⁺].

Step B: Compound ethyl 6-(4-bromo-2,6-difluorobenzyl)amino)-[1,3]dioxolo[4,5-h]quinolin-7-carboxylate (480 mg, 1.03 mmol) was dissolved in methanol/tetrahydrofuran/water (5 mL/4 mL/4 mL), sodium hydroxide (165 mg, 4.14 mmol) was added, and the reaction was carried out at room temperature for 16 h.

After the depletion of raw material as monitored by LCMS, the reaction solution was concentrated, and the pH was adjusted to 3 with 1 M dilute hydrochloric acid under an ice-water bath. The residue was filtered, and the filter cake was collected to give 380 mg of Intermediate 7-3.

MS (ESI) M/Z: 436.9 [M+H⁺].

Step C: Compound 6-(4-bromo-2,6-difluorobenzyl)amino-[1,3]dioxolo[4,5-h]quinolin-7-carboxylic acid (380 mg, 0.87 mmol) was dissolved in N,N-dimethylformamide (5 mL), and diphenylphosphoryl azide (287 mg, 1.04 mmol) and triethylamine (105 mg, 1.04 mmol) were added sequentially to the mixture solution, and the reaction was carried out at 110° C. for 16 h.

After the depletion of raw material as monitored by LCMS, the reaction solution was added with water. The resultant was extracted twice with ethyl acetate, and the organic phases were combined. The combined organic phase was dried with anhydrous sodium sulfate, filtered, and concentrated under reduced pressure, and the residue was purified by silica gel column chromatography to obtain 340 mg of Intermediate 7-4.

MS (ESI) M/Z: 433.9 [M+H⁺].

Step D: Compound 8-(4-bromo-2,6-difluorobenzyl)-6,8-dihydro-7H-[1,3]dioxolo[4,5-h]imidazo[4,5-c]quinolin-7-one (170 mg, 0.39 mmol) was dissolved in N,N-dimethylformamide (5 mL), and benzyl mercaptan (97 mg, 0.79 mmol), N,N-diisopropyl ethylamine (151 mg, 1.17 mmol), 4,5-bis(diphenylphosphino)-9,9-dimethylxanthene (23 mg, 0.04 mmol), and tris(dibenzylideneacetone)dipalladium (36 mg, 0.04 mmol) were added sequentially. After purged three times with nitrogen, the reaction was carried out for 16 h at 80° C.

After the completion of the reaction as monitored by LCMS, the reaction solution was added with water. The resultant was extracted twice with ethyl acetate, and the organic phases were combined. The combined organic phase was dried with anhydrous sodium sulfate, filtered, and concentrated under reduced pressure, and the residue was purified by silica gel column chromatography to obtain 125 mg of Intermediate 7-5.

MS (ESI) M/Z: 478.0 [M+H⁺].

Step E: N-chlorosuccinimide (306 mg, 2.29 mmol) was dissolved in acetonitrile (1 mL), concentrated hydrochloric acid (0.49 mL, 0.59 mmol) was added dropwise to the solution at 0° C., and the reaction was continued at 0° C. for 30 min. Then the compound 8-benzylthio-2,6-difluorobenzyl-6,8-dihydro-7H-[1,3]dioxolo[4,5-h]imidazo[4,5-c]quinolin-7-one (125 mg, 0.28 mmol) was dissolved in acetonitrile (1 mL) and the resultant was added dropwise to the above reaction solution. The reaction was carried out at 0° C. for 1 h.

After the completion of the reaction as monitored by LCMS, the reaction solution was concentrated to remove acetonitrile, and the residue was dissolved in tetrahydrofuran (1 mL), followed by the addition of 0.13 mL of ammonia dropwise, and the reaction was continued at room temperature for 2 h. After the completion of the reaction as monitored by LCMS, the reaction solution was purified by HPLC to give 16 mg of Compound 35.

MS (ESI) M/Z: 435.0 [M+H⁺].

¹H NMR (400 MHz, DMSO-d₆): δ 8.63 (s, 1H), 7.76 (d, J=9.0 Hz, 1H), 7.60 (s, 1H), 7.50-7.44 (m, 2H), 7.36 (d, J=9.0 Hz, 1H), 6.22 (s, 2H), 5.65 (s, 2H).

The following target compounds were prepared with reference to the synthesis method of Example 7 above:

| | | | MS (ESI) |
|---|---|---|---|
| Compound | | | M/Z |
| No | Structural formula | ¹HNMR | [M + H]⁺ |
| 36 | 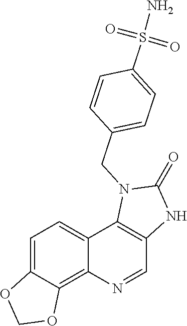 [SMILES: NS(=O)(=O)c1ccc(Cn2c(=O)[nH]c3cnc4c5c(ccc4c32)OCO5)cc1] | (400 MHz, DMSO-d6) δ 8.67 (s, 1H), 7.76 (d, J = 8.4 Hz, 2H), 7.48 (d, J = 9.0 Hz, 1H), 7.41 (d, J = 8.4 Hz, 2H), 7.32 (s, 2H), 7.27 (d, J = 8.9 Hz, 1H), 6.19 (s, 2H), 5.58 (s, 2H). | 399.5 |
| 37 | 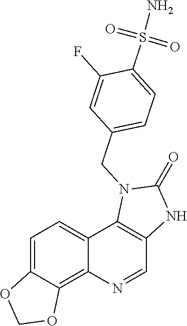 [SMILES: NS(=O)(=O)c1ccc(Cn2c(=O)[nH]c3cnc4c5c(ccc4c32)OCO5)cc1F] | (400 MHz, DMSO-d6) δ 8.67 (s, 1H), 7.72 (t, J = 7.8 Hz, 1H), 7.45 (d, J = 8.9 Hz, 1H), 7.37 (d, J = 10.9 Hz, 1H), 7.29 (d, J = 8.9 Hz, 1H), 7.13 (d, J = 8.1 Hz, 1H), 6.20 (s, 2H), 5.58 (s, 2H). | 417.0 |
| 38 | 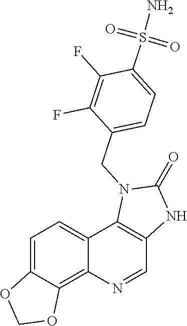 [SMILES: NS(=O)(=O)c1ccc(Cn2c(=O)[nH]c3cnc4c5c(ccc4c32)OCO5)c(F)c1F] | (400 MHz, DMSO-d6) δ 8.68 (s, 1H), 7.56 (s, 2H), 7.49-7.41 (m, 2H), 7.32 (d, J = 8.9 Hz, 1H), 6.91 (t, J = 7.3 Hz, 1H), 6.21 (s, 2H), 5.63 (s, 2H). | 435.1 |
| 39 | 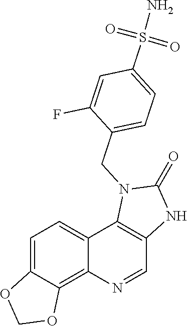 [SMILES: NS(=O)(=O)c1ccc(Cn2c(=O)[nH]c3cnc4c5c(ccc4c32)OCO5)c(F)c1] | (400 MHz, DMSO-d6) δ 11.74 (s, 1H), 8.68 (s, 1H), 7.68 (d, J = 9.7 Hz, 1H), 7.54- 7.39 (m, 4H), 7.30 (d, J = 8.9 Hz, 1H), 7.14 (t, J = 7.7 Hz, 1H), 6.20 (s, 2H), 5.59 (s, 2H). | 417.0 |
| 40 | 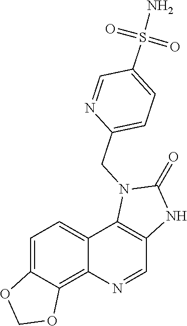 [SMILES: NS(=O)(=O)c1ccc(Cn2c(=O)[nH]c3cnc4c5c(ccc4c32)OCO5)nc1] | (400 MHz, DMSO-d6) δ 8.83 (d, J = 2.3 Hz, 1H), 8.67 (s, 1H), 8.14 (dd, J = 8.3, 2.4 Hz, 1H), 7.55 (d, J = 8.3 Hz, 1H), 7.49 (d, J = 9.0 Hz, 1H), 7.25 (d, J = 8.9 Hz, 1H), 6.20 (s, 2H), 5.568 (s, 2H). | 399.9 |
| 41 | 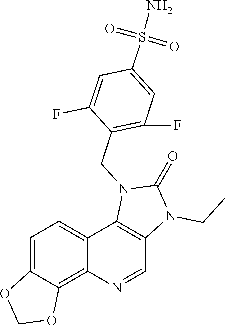 [SMILES: CCn1c(=O)n(Cc2c(F)cc(S(N)(=O)=O)cc2F)c2c3ccc4c(c3ncc21)OCO4] | (400 MHz, DMSO-d6) δ 8.93 (s, 1H), 8.40 (s, 1H), 7.79 (d, J = 8.9 Hz, 1H), 7.47 (d, J = 7.3 Hz, 1H), 7.38 (d, J = 9.0 Hz, 1H), 6.23 (s, 2H), 5.70 (s, 2H), 4.07 (q, J = 7.2 Hz, 2H), 1.29 (t, J = 7.1 Hz, 3H). | 463.0 |
| 53 | 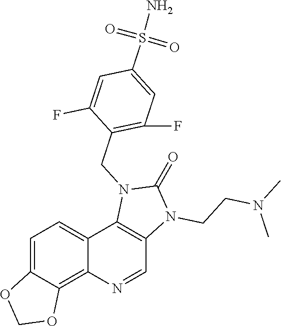 [SMILES: CN(C)CCn1c(=O)n(Cc2c(F)cc(S(N)(=O)=O)cc2F)c2c3ccc4c(c3ncc21)OCO4] | (400 MHz, DMSO-d6) δ 8.89 (s, 1H), 7.77 (d, J = 8.9 Hz, 1H), 7.59 (s, 3H), 7.45 (d, J = 7.4 Hz, 2H), 7.37 (d, J = 8.9 Hz, 1H), 6.22 (s, 2H), 5.70 (s, 2H), 4.10 (t, J = 6.2 Hz, 2H), 2.59 (t, J = 6.2 Hz, 2H), 2.17 (s, 6H). | 506.1 |

### Example 8: (8-(3-fluoro-4-(S-methylsulfonyl)benzyl)-6,8-dihydro-7H-[1,3]dioxolo[4,5-h]imidazo[4,5-c]quinolin-7-one)

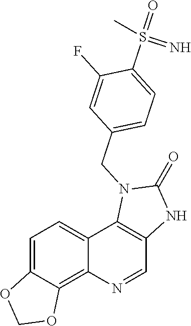 [SMILES: CS(=N)(=O)c1ccc(Cn2c(=O)[nH]c3cnc4c5c(ccc4c32)OCO5)cc1F]

### Reaction Route:

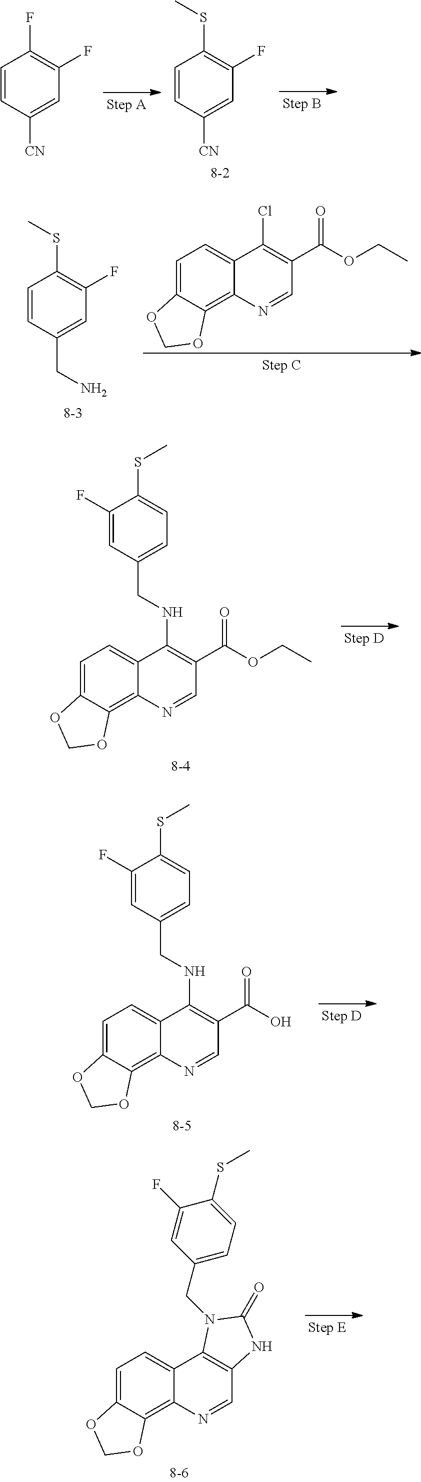

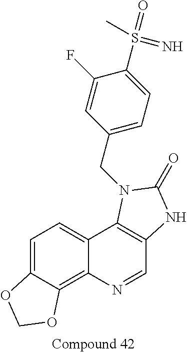 [SMILES: CS(=N)(=O)c1ccc(Cn2c(=O)[nH]c3cnc4c5c(ccc4c32)OCO5)cc1F]

Step A: Compound 3,4-difluorobenzonitrile (7 g, 50.3 mmol) was dissolved in N,N-dimethylformamide (70 mL), sodium thiomethoxide (3.88 g, 55.3 mmol) was added, and the reaction was carried out at 0° C. for 1 h. After the depletion of raw material as monitored by LCMS, the reaction solution was added with 200 mL of water, and the residue was filtered. The filter cake was collected to give 7.3 g of Intermediate 8-1.

Step B: Compound 3-fluoro-4-(methylthio)benzonitrile (1 g, 5.99 mmol) was dissolved in methanol (10 mL), and Raney nickel (0.2 g) was added. After nitrogen displacement, hydrogen was passed through and the reaction was carried out at room temperature overnight. The reaction solution was filtered to remove catalyst, and the filtrate was concentrated to give 850 mg of Intermediate 8-2.

Step C: Compound ethyl 6-chloro-[1,3]dioxolo-[4,5-h]quinolin-7-formate (220 mg, 0.79 mmol) was dissolved in N,N-dimethylformamide (5 mL), and (3-fluoro-4-(methylthio)phenyl)methylamine (405 mg, 2.37 mmol) and N,N-diisopropylethylamine (241 mg, 2.39 mmol) were added to the solution sequentially. The reaction was carried out at 100° C. for 2 h under microwave. After the depletion of raw material as monitored by LCMS, the reaction solution was added with water, and the resultant was extracted with ethyl acetate. The organic phase were dried and concentrated, and the residue was purified by silica gel column chromatography to obtain 300 mg of Intermediate 8-3.

MS (ESI) M/Z: 415.0 [M+H⁺].

Step D: Compound ethyl 6-(3-fluoro-4-(methylthio)benzyl)amino)-[1,3]dioxolo[4,5-h]quinolin-7-carboxylate (300 mg, 0.72 mmol) was dissolved in methanol/tetrahydrofuran/water (2 mL/2 mL/2 mL), sodium hydroxide (144 mg, 3.6 mmol) was added, and the reaction was carried out at 45° C. for 2 h. After the depletion of raw material as monitored by LCMS, the reaction solution was concentrated. The pH was adjusted to 3 with 1 M dilute hydrochloric acid under an ice-water bath, the residue was filtered, and the filter cake was collected to give 220 mg of Intermediate 8-4.

MS (ESI) M/Z: 387.0 [M+H⁺].

Step E: Compound 6-(3-fluoro-4-(methylthio)benzyl)amino)-[1,3]dioxolo[4,5-h]quinolin-7-carboxylic acid (220 mg, 0.57 mmol) was dissolved in N,N-dimethylformamide (5 mL), and diphenylphosphoryl azide (188 mg, 0.68 mmol) and triethylamine (69 mg, 0.68 mmol) were added sequentially to the mixture solution. After three times of nitrogen displacement, the reaction was carried out at 110° C. for 16 h. After the depletion of raw material as monitored by LCMS, the reaction solution was added with water and extracted with ethyl acetate. The organic phase were dried and concentrated, and the residue was purified by silica gel column chromatography to obtain 150 mg of Intermediate 8-5.

MS (ESI) M/Z: 384.0 [M+H⁺].

Step F: Compound 8-(3-fluoro-4-(methylthio)benzyl)-6,8-dihydro-7H-[1,3]dioxolo[4,5-h]imidazo[4,5-c]quinolin-7-one (150 mg, 0.39 mmol) was dissolved in ethanol (2 mL), and (diacetoxyiodo)benzene (502 mg, 1.56 mmol) and ammonium acetate (150 mg, 1.95 mmol) were added sequentially, and the solution was stirred at room temperature for 2 h. LCMS monitoring showed there existed residual raw material, and the reaction solution was purified by HPLC (NH₃H₂O) to give 32.03 mg of Compound 42.

MS (ESI) M/Z: 415.00 [M+H⁺].

¹H NMR (400 MHz, DMSO-d₆) δ 11.74 (s, 1H), 8.6 (s, 1H), 7.81 (t, J=7.8 Hz, 1H), 7.50 (d, J=8.9 Hz, 1H), 7.38 (dd, J=10.9, 1.6 Hz, 1H), 7.31 (d, J=9.0 Hz, 1H), 7.21 (dd, J=8.2, 1.6 Hz, 1H), 6.21 (s, 2H), 5.60 (s, 2H), 4.68 (s, 1H), 3.14 (s, 3H).

The following target compounds were prepared with reference to the synthesis method of Example 8 above. Among them, Compound 43 was separated by chiral HPLC under the following conditions: chiral column: Daicel AD (25*250 mm, 10 μm); mobile phase: CO₂/EtOH [0.5% NH₃ (7 M in MeOH)]=60/40; 3.93 mg of Compound 43-P1 (time of peak occurrence; 1.623 min) and 3.46 mg of Compound 43-P2 (time of peak occurrence: 1.830 min) were obtained. Compound 43-P1 and Compound 43-P2 are enantiomers of each other, and the absolute configurations of which were not determined.

| | | | MS (ESD) |
|---|---|---|---|
| Compound | | | M/Z |
| No | Structural formoula | ANMR | [M + H]⁺ |
| 43 | 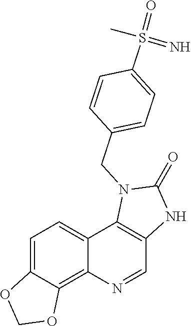 [SMILES: CS(=N)(=O)c1ccc(Cn2c(=O)[nH]c3cnc4c5c(ccc4c32)OCO5)cc1] | (400 MHz, Methanol-d4) δ 8.65 (s, 1H), 7.98 (d, J = 8.5 Hz, 2H), 7.58-7.49 (m, 3H), 7.18 (d, J = 8.9 Hz, 1H), 6.20 (s, 2H), 5.71 (s, 2H), 3.11 (s, 3H). | 397.0 |
| 43-P1 | 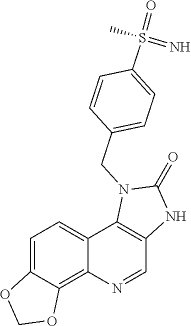 [SMILES: C[S@@](=N)(=O)c1ccc(Cn2c(=O)[nH]c3cnc4c5c(ccc4c32)OCO5)cc1] | (400 MHz, Methanol-d4) δ 8.65 (s, 1H), 7.98 (d, J = 8.5 Hz, 2H), 7.62-7.47 (m, 3H), 7.18 (d, J = 9.0 Hz, 1H), 6.20 (s, 2H), 5.71 (s, 2H), 3.11 (s, 3H). | 396.9 |
| 43-P2 | 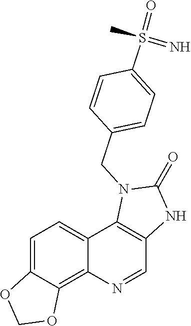 [SMILES: C[S@](=N)(=O)c1ccc(Cn2c(=O)[nH]c3cnc4c5c(ccc4c32)OCO5)cc1] | (400 MHz, Methanol-d4) δ 8.65 (s, 1H), 7.98 (d, J = 8.5 Hz, 2H), 7.65-7.45 (m, 3H), 7.18 (d, J = 8.9 Hz, 1H), 6.20 (s, 2H), 5.71 (s, 2H), 3.11 (s, 3H). | 396.9 |
| 44 | 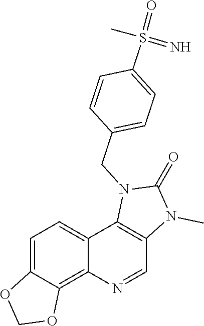 [SMILES: Cn1c(=O)n(Cc2ccc(S(C)(=N)=O)cc2)c2c3ccc4c(c3ncc21)OCO4] | (400 MHz, DMSO-d6) δ 8.88 (s, 1H), 7.51 (d, J = 9.0 Hz, (1H), 7.44 (d, J = 8.3 Hz, 2H), 7.27 (d, J = 8.9 Hz, 1H), 6.19 (s, 2H), 5.63 (s, 2H), 3.58 (s, 3H), 3.00 (s, 3H). | 411.0 |
| 45 | 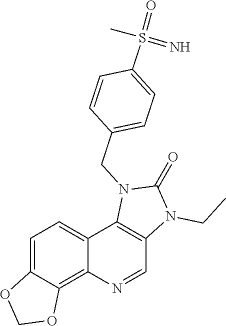 [SMILES: CCn1c(=O)n(Cc2ccc(S(C)(=N)=O)cc2)c2c3ccc4c(c3ncc21)OCO4] | (400 MHz, DMSO-d6) δ 8.93 (s, 1H), 7.85 (d, J = 8.4 Hz, 2H), 7.51 (d, J = 8.9 Hz, 1H), 7.42 (d, J = 8.4 Hz, 2H), 7.27 (d J = 9.0 Hz, 1H), 6.19 (s, 2H), 5.63 (s, 2H), 4.12 (q, J = 7.1 Hz, 2H), 3.00 (s, 3H), 1.34 (t, J = 7.1 Hz, 3H). | 425.1 |

### Example 9: 3,5-difluoro-4-(7-oxo-6,7-dihydro-8H-[1,3]dioxolo[4,5-h]imidazo[4,5-c]quinolin-8-yl)methyl)phenylboronic acid

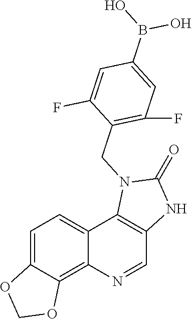 [SMILES: O=c1[nH]c2cnc3c4c(ccc3c2n1Cc1c(F)cc(B(O)O)cc1F)OCO4]

### Reaction Route:

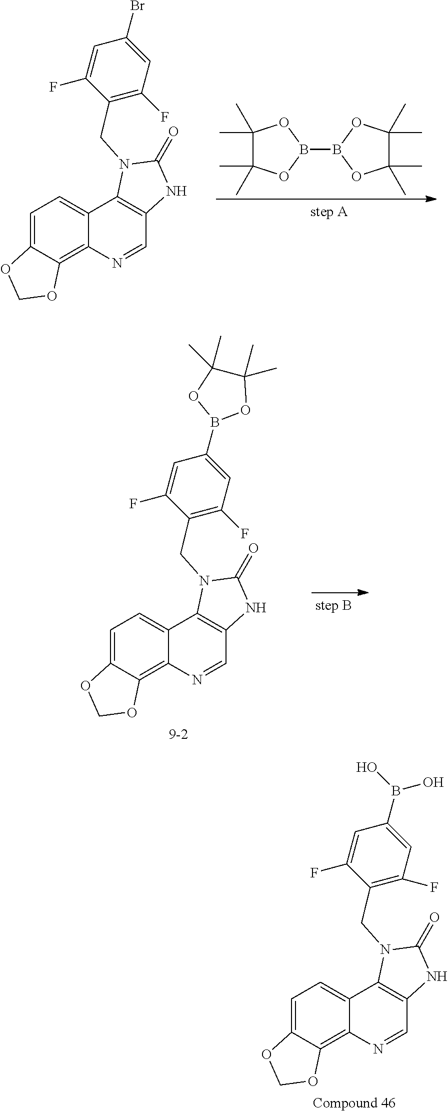

Step A: Compound 8-(4-bromo-2,6-difluorobenzyl)-6,8-dihydro-7H-[1,3]dioxolo[4,5-h]imidazo[4,5-c]quinolin-7-one (120 mg, 0.28 mmol) was dissolved in 1,4-dioxane (5 mL), and bis(pinacolato)diboron (107 mg, 0.42 mmol), potassium acetate (82 mg, 0.84 mmol), and [1,1′-bis(diphenylphosphino)ferrocene]palladium dichloride (29 mg, 0.04 mmol) were added sequentially. After three times of nitrogen displacement, the reaction was carried out for 16 h at 100° C. After completion of the reaction as monitored by LCMS, the reaction solution was added with water and extracted twice with ethyl acetate. The organic phases were combined, and the combined organic phase was dried with anhydrous sodium sulfate, filtered, and concentrated under reduced pressure, and the residue was purified by silica gel column chromatography to obtain 87 mg of Intermediate 9-2.

MS (ESI) M/Z: 482.2 [M+H⁺].

Step B: 8-(2,6-difluoro-4-(4,4,5,5-tetramethyl-1,3,2-dioxaborolan-2-yl)benzyl)-6,8-dihydro-7H-[1,3]dioxolo[4,5-h]imidazo[4,5-c]quinolin-7-one (88 mg, 0.18 mmol) was dissolved in acetone (2 mL) and water (1 mL), and sodium periodate (117 mg, 0.54 mmol) and ammonium acetate (28 mg, 0.36 mmol) were added sequentially, and the reaction was carried out at room temperature for 3 h. After completion of the reaction as monitored by LCMS, the reaction solution was purified by HPLC to give 15 mg of Compound 46.

MS (ESI) M/Z: 400.10 [M+H⁺].

¹H NMR (400 MHz, DMSO-d6+D₂O) δ 11.58 (s, 1H), 8.62 (s, 1H), 8.38 (s, 2H), 7.68 (d, J=9.01 Hz, 1H), 7.37-7.25 (m, 2H), 6.20 (s, 2H), 5.61 (s, 2H).

The following target compounds were prepared with reference to the synthesis method of Example 9 above:

| | | | MS (ESD) |
|---|---|---|---|
| Compound | | | M/Z |
| No | Structural formula | ¹HNMR | [M + H]⁺ |
| 47 | 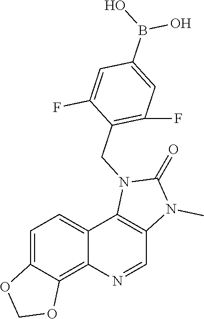 [SMILES: Cn1c(=O)n(Cc2c(F)cc(B(O)O)cc2F)c2c3ccc4c(c3ncc21)OCO4] | (400 MHz, DMSO-d6) δ 8.85 (s, 1H), 8.38 (s, 1H), 7.72 (d, J = 9.1 Hz, 1H), 7.38-7.30 (m, 2H), 6.22 (s, 2H), 5.64 (s, 2H), 3.53 (s, 3H). | 414.0 |
| 48 | 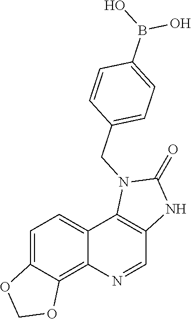 [SMILES: O=c1[nH]c2cnc3c4c(ccc3c2n1Cc1ccc(B(O)O)cc1)OCO4] | (400 MHz, DMSO-d6) δ 11.87 (s, 1H), 8.70 (s, 1H), 7.72 (d, J = 8.1 Hz, 2H), 7.56 (d, J = 9.0 Hz, 1H), 7.31 (d, J = 9.0 Hz, 1H), 7.18 (d, J = 7.7 Hz, 2H), 6.24 (s, 2H), 5.52 (s, 2H). | 364.0 |

### Example 10: (2-fluoro-4-((7-methyl-8H-[1,3]dioxolo[4,5-h]imidazo[4,4-c]quinolin-8-yl)methyl)phenyl)phosphonic acid

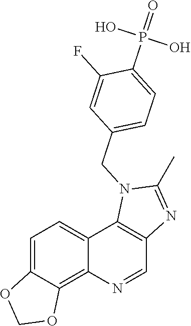 [SMILES: Cc1nc2cnc3c4c(ccc3c2n1Cc1ccc(P(=O)(O)O)c(F)c1)OCO4]

### Reaction Route:

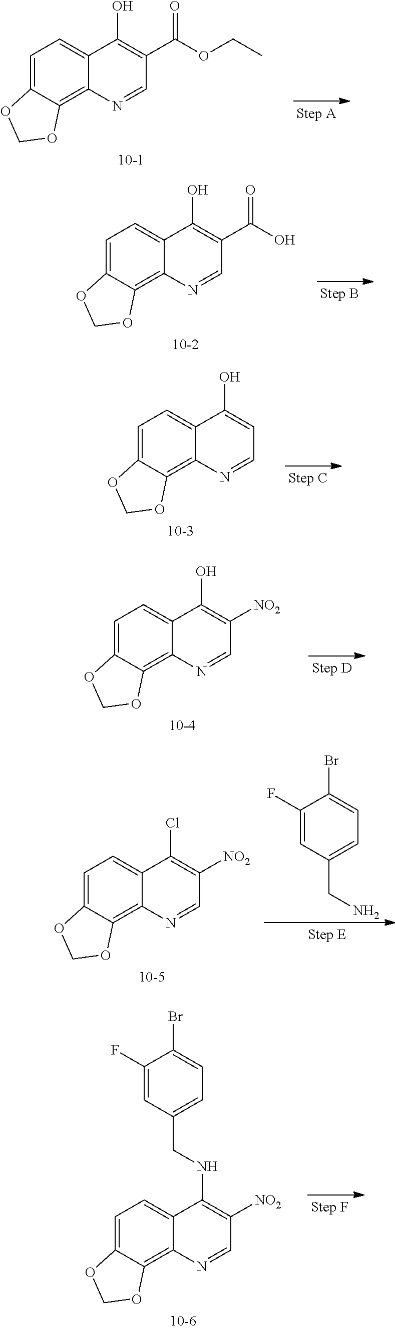

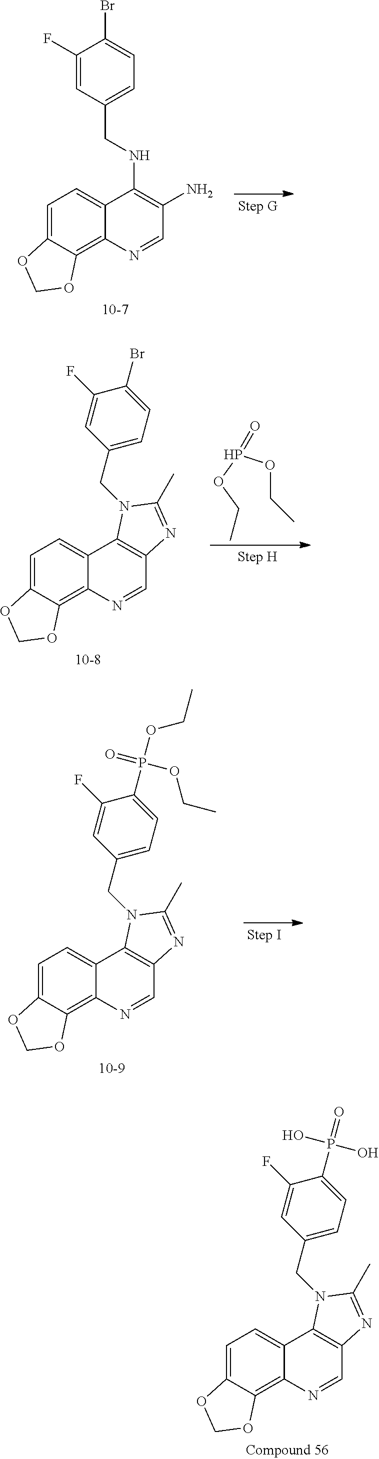

### Operating Procedure:

Step A: Ethyl 6-hydroxy-[1,3]dioxolo[4,5-h]quinolin-7-carboxylate (5 g, 19.14 mmol) was dissolved in tetrahydrofuran/water/ethanol (50 mL/50 mL/50 mL) at room temperature. Subsequently, sodium hydroxide (5.36 g, 134 mmol) was added to the solution at room temperature. The reaction solution was then heated to 60° C. and stirred for 16 h. After the depletion of raw material as monitored by LCMS the reaction solution was cooled to room temperature, the pH was adjusted to 3 by adding dilute hydrochloric acid (2 mol/L), and a white solid was precipitated. The mixture was filtered and the filter cake was washed twice with water (20 mL) and dried to give 3.5 g of Intermediate 10-2.

MS (ESI) M/Z: 234.0 [M+H⁺].

Step B: 6-hydroxy-[13]dioxolo[4,5-h]quinolin-7-carboxylic acid (3.5 g, 15 mmol) was dissolved in diphenyl ether (150 mL) at room temperature. The reaction solution was then stirred at 250° C. for 4 h. After the depletion of raw material as monitored by LCMS, the reaction solution was cooled to about 40° C., and then petroleum ether (150 mL) was added to the reaction solution. The mixture was cooled down to room temperature while stirring, and was filtered after stirring for one hour. The resulting filter cake was washed three times with petroleum ether (100 mL) and the filter cake was dried to give 2.9 g of Intermediate 10-3.

MS (ESI) M/Z: 190.0 [M+H⁺].

Step C: [1,3]dioxolo[4,5-h]quinolin-6-ol (2.9 g, 15.32 mmol) was dissolved in propionic acid (30 mL) at room temperature. The reaction solution was then heated to 140° C. and stirred for 0.5 h. Half an hour later fuming nitric acid (965 mg, 15.32 mmol) was added and stirring was continued for 16 h. After the depletion of raw material as monitored by LCMS, the reaction solution was cooled to room temperature and filtered. The resulting filter cake was washed three times with ethyl acetate (30 mL) and the filter cake was dried to give 2.58 g of Intermediate 10-4.

MS (ESI) M/Z: 234.9 [M+H⁺].

Step D: 7-nitro-[1,3]dioxolo[4,5-h]quinolin-6-ol (1 g, 4.27 mmol) and N,N-diisopropylethylamine (1 mL) were dissolved in trichloromethane (10 mL) at room temperature. Subsequently, phosphorous oxychloride (2 mL) was slowly added to the solution at room temperature, and the resulting mixed turbid reaction solution was stirred at 90° C. for 16 h, and the solution became clear and transparent. After the depletion of raw material as monitored by TLC, the reaction solution was concentrated under reduced pressure, and the crude target compound was purified by column chromatography (dichloromethane:ethyl acetate=1:1) to give 220 mg of Intermediate 10-5.

Step E: 6-Chloro-7-nitro-[1,3]dioxolo[4,5-h]quinoline (200 mg, 0.792 mmol) and (4-bromo-3-fluorophenyl)formamide (161.6 mg, 0.792 mmol) were dissolved in acetonitrile (8 mL) at room temperature. Subsequently, triethylamine (0.33 mL, 2.376 mmol) was added to the solution at room temperature, and the resulting mixed reaction solution was stirred at 80° C. for 5 h. After the depletion of raw material as monitored by LCMS, petroleum ether/ethyl acetate/dichloromethane (10 mL/5 mL/5 mL) were added to the reaction solution, and the mixture was stirred at room temperature for 1 h and then filtered. The resulting filter cake was washed three times with dichloromethane/ethyl acetate (3 mL/3 mL) and the filter cake was dried to give 300 mg of Intermediate 10-6.

MS (ESI) M/Z: 419.8 [M+H⁺].

Step F: N-(4-bromo-3-fluorobenzyl)-7-nitro-[1,3]dioxolo[4,5-h]quinolin-6-amine (190 mg, 0.452 mmol) was dissolved in methanol (5 mL) at room temperature. Subsequently, Raney nickel (20 mg) was added to the solution at room temperature and the resulting mixed reaction solution was stirred at room temperature under hydrogen atmosphere for 2.5 h. After the depletion of raw material as monitored by LCMS, the reaction solution was filtered and concentrated under reduced pressure, and the crude product was purified by plate chromatography to give 80 mg of Intermediate 10-7.

MS (ESI) M/Z: 389.8 [M+H⁺].

Step G: N-6-(4-bromo-3-fluorobenzyl)-[1,3]dioxolo[4,5-h]quinolin-6,7-diamine (80 mg, 0.205 mmol) and pyridine hydrochloride (0.46 mg, 0.004 mmol) were dissolved in anhydrous tetrahydrofuran (6 mL) at room temperature. Subsequently, triethyl orthoacetate (66.51 mg, 0.410 mmol) was added to the solution at room temperature, and the resulting mixed reaction solution was stirred at 100° C. for 16 h. After the depletion of raw material as monitored by LCMS, the reaction solution was concentrated under reduced pressure, and the crude product was purified by plate chromatography to give 53 mg of Intermediate 10-8.

MS (ESI) M/Z: 413.8 [M+H⁺].

Step H: 8-(4-bromo-3-fluorobenzyl)-7-methyl-8H-[1,3]dioxolo[4,5-h]imidazo[4,4-c]quinoline (53 mg, 0.128 mmol) was dissolved in N,N-dimethylformamide (1.8 mL) at room temperature. Subsequently, diethyl phosphite (35.4 mg, 0.256 mmol), [1,1′-bis(diphenylphosphino)ferrocene]palladium(II) dichloride dichloromethane complex (10.6 mg, 0.013 mmol), and triethylamine (38.8 mg, 0.384 mmol) were added to the solution, and the resulting mixed reaction solution was stirred for 4.5 h at 100° C. under microwave. After the depletion of raw material as monitored by LCMS, the mixture was stirred and poured into water (20 mL), and the resultant was extracted three times with ethyl acetate (50 mL). The combined organic layer was washed three times with saturated saline (20 mL), dried over anhydrous sodium sulfate, filtered, and concentrated to obtain a crude product, which was purified by plate chromatography to give 35 mg of Intermediate 10-9.

MS (ESI) M/Z: 471.8 [M+H]⁺.

Step I: Diethyl (2-fluoro-4-((7-methyl-8H-[1,3]dioxolo[4,5-h]imidazo[4,4-c]quinolin-8-yl)methyl)phenyl)phosphonate (35 mg, 0.074 mmol) was dissolved in a solution of acetonitrile (3 mL) and trimethylbromosilane (0.5 mL) at room temperature. Subsequently, the reaction solution was stirred at room temperature for 16 h. After the depletion of raw material as monitored by LCMS, the reaction solution was added to methanol (30 mL) and the resultant was stirred for 1 h. The resultant was concentrated under reduced pressure, and the crude target compound was purified by preparative high performance liquid chromatography to give 2.36 mg of Compound 54.

MS (ESI) M/Z: 415.8 [M+H⁺].

¹H NMR (400 MHz, DMSO-d₆): δ 9.10 (s, 1H), 7.67-7.55 (m, 2H), 7.31 (d, J=8.8 Hz, 1H), 7.02-6.93 (m, 1H), 6.85-6.79 (m, 1H), 6.22 (s, 2H), 5.95 (s, 2H), 2.61 (s, 3H).

The following target compounds were prepared with reference to the synthesis method of Example 10 above:

| | | | MS (ESI) |
|---|---|---|---|
| Compound | | | M/Z |
| No | Structural formula | HNMR | [M + H]⁺ |
| 62 | 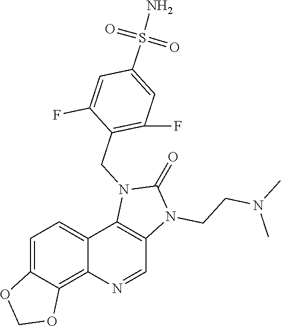 [SMILES: CN(C)CCn1c(=O)n(Cc2c(F)cc(S(N)(=O)=O)cc2F)c2c3ccc4c(c3ncc21)OCO4] | (400 MHz, DMSO-d6) δ 8.89 (s, 1H), 7.77 (d, J = 8.9 Hz, 1H), 7.59 (s, 3H), 7.45 (d, J = 7.4 Hz, 2H), 7.37 (d, J = 8.9 Hz, 1H), 6.22 (s, 2H), 5.70 (s, 2H), 4.10 (t, J = 6.2 Hz, 2H), 2.59 (t, J = 6.2 Hz, 2H), 2.17 (s, 6H). | 506.3 |
| 63 | 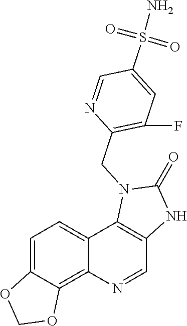 [SMILES: NS(=O)(=O)c1cnc(Cn2c(=O)[nH]c3cnc4c5c(ccc4c32)OCO5)c(F)c1] | (400 MHz, DMSO-d6) δ 8.65 (s, 1H), 8.54 (s, 1H), 8.12 (dd, J = 9.3. 1.8 Hz, 1H), 7.51 (d, J = 9.0 Hz, 1H), 7.25 (d, J = 8.9 Hz, 1H), 6.20 (s, 2H), 5.77 (s, 2H). | 417.9 |
| 64 | 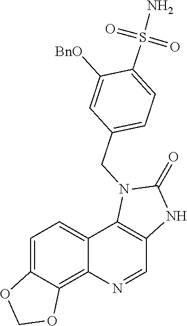 [SMILES: NS(=O)(=O)c1ccc(Cn2c(=O)[nH]c3cnc4c5c(ccc4c32)OCO5)cc1OCc1ccccc1] | (400 MHz, DMSO-d6) δ 8.87 s, 1H), 7.71 (d, J = 8.1 Hz, 1H), 7.58 (d, J = 9.2 Hz, 1H), 7.36 (d, J = 9.1 Hz, 1H), 7.29- 7.17 (m, 2H), 7.19-7.01 (m, 3H), 6.96 (d, J = 8.1 Hz, 1H), 6.88 (s, 1H), 6.44 (s, 2H), 5.57 (s, 2H), 5.27 (s, 2H). | 505.0 |

### II. Biological Activity

### (I) In Vitro Enzyme Activity Assay

### 1. Experimental Materials

AMP-Glo Assay Kit (PROMEGA, V5012), ENPP-1-IN-1 (MCE, HY-129490), white opaque 384-well plate (Perkin Elmer, 6008289), Mammalian (non-canonical) CDN, cyclic[g(2′,5′)pA(3′,5′)p](referred to as cGAMP hereinafter, Invivogen, tlrl-nacga23-5), ENPP1 Protein Human HEK293 (Biovendor, AP-18-081).

### 2. Experimental Method

1) Preparing 1× reaction buffer (Tris buffer), 1× enzyme buffer, and 1× substrate buffer;
2) diluting the compounds using Echo550 and supplementing with DMSO;
3) pre-loading 2.5 μL of enzyme solution on a reaction plate (adding 2.5 μL of buffer instead as negative control) and incubating it at room temperature;
4) adding 2.5 μL of substrate solution to the system and incubating it at room temperature;
5) adding 5 μL of AMP-Glo solution that has been allowed to cooled down to room temperature, centrifuging and reacting at room temperature,
6) adding 10 μL of AMP Detection solution at room temperature, centrifuging and reacting at room temperature; and
7) reading the plate using EnVision.

It was determined that the compounds of the present invention have excellent inhibitory effects on ENPP1, with their IC₅₀ values generally lower than 2 μM. The IC₅₀ values of some compounds of the present invention are lower than 1 μM, and the IC₅₀ values of more excellent compounds of the present invention are lower than 0.5 μM, or even lower than 0.3 μM. The inhibition results of some compounds of the present invention on ENPP1 are shown in Table 1.

TABLE 1
Enzymatic inhibition results

| | human ENPP1 | | human ENPP1 |
|---|---|---|---|
| | enzymatic | | enzymatic |
| | assay | | assay |
| Compound | (cGAMP) (nM) | Compound | (cGAMP) (nM) |
| Compound 1 | A | Compound 2 | A |
| Compound 3 | A | Compound 4 | A |
| Compound 5-P1 | A | Compound 5-P2 | A |
| Compound 6 | A | Compound 7 | A |
| Compound 8 | B | Compound 9 | A |
| Compound 10 | A | Compound 11 | A |
| Compound 12 | A | Compound 13 | A |
| Compound 14 | A | Compound 15 | A |
| Compound 16 | A | Compound 17 | A |
| Compound 18 | A | Compound 19 | A |
| Compound 20 | A | Compound 21 | A |
| Compound 22 | A | Compound 23 | A |
| Compound 24 | A | Compound 25 | A |
| Compound 26 | A | Compound 27 | A |
| Compound 28 | A | Compound 29 | A |
| Compound 30 | A | Compound 31 | A |
| Compound 32 | A | Compound 33 | A |
| Compound 34 | A | Compound 35 | A |
| Compound 36 | A | Compound 37 | A |
| Compound 38 | A | Compound 39 | A |
| Compound 40 | A | Compound 41 | A |
| Compound 42 | A | Compound 43-P1 | B |
| Compound 43-P2 | A | Compound 44 | A |
| Compound 45 | A | Compound 46 | A |
| Compound 47 | A | Compound 48 | A |
| Compound 49 | A | Compound 50 | A |
| Compound 51 | A | Compound 52 | A |
| Compound 53 | A | Compound 54 | A |
| Compound 55 | A | Compound 56 | A |
| Compound 57 | C | Compound 58 | A |
| Compound 59 | A | Compound 60 | A |
| Compound 61 | A | Compound 62 | A |
| Compound 63 | A | / | / |

Note:
The IC₅₀ data of the inhibition activity of the compounds of the present invention for ENPP1 are presented in Table 1. The compounds with IC₅₀ less than 10 nM are identified by A, compounds with IC₅₀ between 10 nM and 50 nM are identified by B, compounds with IC₅₀ between 50 nM and 100 nM are identified by C, compounds with IC₅₀ between 100 nM and 1000 nM are identified by D, and compounds with IC₅₀ greater than 1000 nM are identified by E.
CONCLUSION: The compounds of the present invention have excellent inhibitory effects on ENPP1.

### (II) Cell Activity Test 1

### 1. Experimental Method

MDA-MB-231 cells were digested and suspended, the cell density was adjusted to 4.5×10⁵ cells per milliliter, and the cells were seeded in 384 microtiter plates at 50 microliters per well. The cells were incubated in an incubator at 37° C. with 1% CO₂ for 24 h. After 24 h, the cell plates were taken out, the cell culture medium was pipetted and washed once with 50 μL of PBS. Then 20 μL of 1640 medium free of phenol red and serum was added per well, and 2× of compound was added with Tecan, with 1:3 dilution. The compounds were pre-incubated for 1 hour. Afterwards, the reaction was initiated by adding a substrate mixture (50 μL system, 0.5 mM pNP-AMP dissolved in 1640 medium free of phenol red and serum) at a final substrate concentration of 0.25 mM. Absorbance values were read at 405 nM in a microplate reader after 5 hours of reaction, 4-Parameter Logistic Curve model was applied to calculate the IC % values of the compounds, taking positive compounds as 100% inhibition and DMSO as 0% inhibition.

### 2. Experimental Results

Compared with the compounds disclosed in the prior art, the compounds of the present application have obvious cell proliferation inhibitory activities. The specific experimental data are shown in the table below.

| Compound | Compound 1 | Control Compound |
|---|---|---|
| MDA-MB-231 cell | 1.12 | 9.78 |
| biochemical IC50 | | |
| (nM) | | |

### (III) Cell Activity Test 2

### 1. Experimental Materials

THP1-Dual Cells NF-κB-SEAP and IRF-Lucia luciferase Reporter Monocytes cells (Invivogen, thpd-nfis), MDA-MB-231 cells (ATCC, CRM-HTB-26), RPMI-1640 medium (Gibco, 11875-093), FBS (Gibco, 10099), penicillin-streptomycin (Gibco, 15070063), HEPES (ThermoFisher, 15630080), L-glutamine (ThermoFisher, 25030081), Normocin-Antimicrobial Reagent (InVivoGen, ant-nr-1), Zeocin (Invivogen, ant-zn-1), Blasticidin (Invivogen, ant-bl-1), Mammalian (non-canonical) CDN, cyclic [G(2′,5′)pA(3′,5′)p] (referred to as cGAMP hereinafter, Invivogen, tlrl-nacga23-1), QUANTI-Luc (InvivoGen, rep-qlc2), Multimode Plate Reader (PerkinElmer, EnVision 2105 Alpha 680 nm Laser), and conventional cell culture consumables (NEST).

### 2. Experimental Method

### 1) Preparation of Culture Medium:

MDA-MB-231 Cell Growth Medium

Leibovitz's L-15 medium, containing 10% thermal deactivation (30 min at 56 degrees Celsius) treated FBS, 1× penicillin-streptomycin.
THP1-Dual Cells NF-κB-SEAP and IRF-Lucia luciferase Reporter Monocytes Cell Growth Medium

RPMI-1640 medium, containing 10% thermal deactivation (30 min at 56 degrees Celsius) treated FBS, 25 mM HEPES, 2 mM L-glutamine, 100 g/mL Normocin, 1× penicillin-streptomycin.
THP1-Dual Cells NF-κB-SEAP and IRF-Lucia luciferase Reporter Cell Maintenance Medium

On the basis of THP1-Dual Cells NF-κB-SEAP and IRF-Lucia luciferase Reporter Growth Medium, 10 μg/mL Blasticidin and 100 μg/mL Zeocin were added. This medium was used alternately from Growth medium.
Test medium

RPMI-1640 medium, containing 10% thermal deactivation (30 min at 56 degrees Celsius) treated FBS, 25 mM HEPES, 2 mM L-glutamine, 1× penicillin-streptomycin.

2) Preparation of cGAMP Working Solution

10× cGAMP concentrated solution (10 μM) was prepared using Test medium.

### 3) Inoculation of Cell

An appropriate amount of MDA-MB-231 cells in logarithmic growth phase was taken and centrifuged at 300×g for 5 minutes, and the supernatant was removed. The cells were re-suspended with MDA-MB-231 cell growth medium and counted, and the cell density was adjusted to 6×10⁴ cells/mL. The cells were inoculated at 50 μL/well in a 384-well plate and incubated overnight.

### 4) Treatment of Cells

The compounds were added to the cells at specific concentrations for 30 minutes for pretreatment, followed by the addition of 5.5 μL 10× cGAMP concentrated solution, and the mixture was shaken well and placed in a cell incubator for 24 hours.

### 5) Transfer of Supernatant

35 μL of supernatant from MDA-MB-231 cells was transferred to a new 384-well plate, followed by the addition of 35 μL of THP1-Dual Cells NF-κB-SEAP and IRF-Lucia luciferase Reporter Monocytes Cell Suspension (Test medium) at a concentration of 1-10⁶, and the mixture was co-incubated for 24 hours.

### 6) Read the Luciferase

After 24 hours, the resulting was centrifuged at 300×g for 5 minutes, 10 μL of cell supernatant was transferred to a new 384-well plate with a white opaque bottom, followed by the addition of 25 μL of QUANTI-Luc reaction solution to each well. They were shaken well for 1 minute away from light and the plate was read immediately.

### 3. Experimental Results

Compared with the compounds disclosed in the prior art, the compounds of the present application have obvious cell proliferation inhibitory activities. The specific experimental data are shown in the table below:

| Compound | Compound 1 | Control Compound |
|---|---|---|
| MDA-MB-231 CM- | 3.5 | 14.6 |
| THP1 EC₅₀ (nM) | | |

### Attachment: Structure of Control Compound

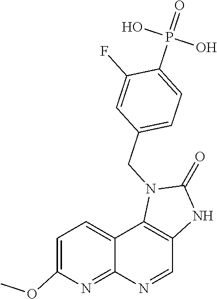 [SMILES: COc1ccc2c(ncc3[nH]c(=O)n(Cc4ccc(P(=O)(O)O)c(F)c4)c32)n1]

1. A compound represented by formula (I-b): 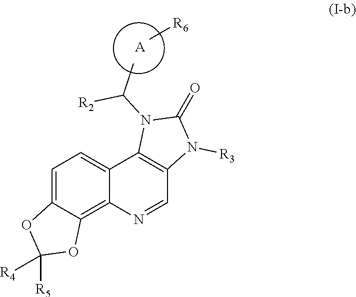
or a stereoisomer, a tautomer or a pharmaceutically acceptable salt thereof, wherein,
ring A is selected from the group consisting of aryl and 5- to 6-membered heteroaryl, wherein the aryl or heteroaryl is optionally substituted by 1 to 4 R₁;
R₁ is selected from the group consisting of hydrogen, halogen, C₁₋₄ alkyl, C₁₋₄ alkoxy, and aryl-C₁₋₄ alkoxy;
R₂ is selected from the group consisting of hydrogen and C₁₋₄ alkyl;
R₃ is hydrogen or is selected from the group consisting of C₁₋₄ alkyl, C₃₋₆ cycloalkyl, 5- to 6-membered heterocycloalkyl, —C₁₋₄ alkylene-C₃₋₆ cycloalkyl, —C₁₋₄ alkylene-(5- to 6-membered heterocycloalkyl), —C₁₋₄ alkylene-CO—C₁₋₄ alkylene, and phenyl, which are optionally substituted by 1 to 3 R_{d}, wherein, each carbon atom in C₁₋₄ alkylene is optionally replaced by —N—, —O—, or —S—, and the S atom is optionally oxidized; R_{d} is selected from the group consisting of halogen, C₁₋₄ alkyl, and —NR_{da}R_{db}, and the C cycloalkyl is optionally substituted by 1 to 4 Re; and wherein the R_{da} and R_{db} are each independently selected from the group consisting of hydrogen and C₁₋₄ alkyl, and Re is selected from the group consisting of halogen and C₁₋₄ alkyl;
R₄ and R₅ are each independently selected from the group consisting of hydrogen and C₁₋₄ alkyl; and
R₆ is a hydrophilic group.

2. A compound represented by formula (I-a): 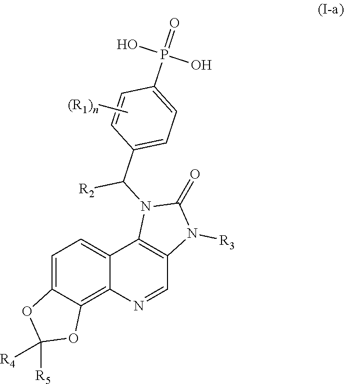
or a stereoisomer, a tautomer or a pharmaceutically acceptable salt thereof, wherein,
R₁ is selected from the group consisting of hydrogen, halogen, C₁₋₄ alkyl, C₁₋₄ alkoxy, and aryl-C₁₋₄ alkoxy;
R₂ is selected from the group consisting of hydrogen and C₁₋₄ alkyl;
R₃ is hydrogen or is selected from the group consisting of C₁₋₄ alkyl, —C₁₋₄ alkylene-CO—C₁₋₄ alkylene, and phenyl, which are optionally substituted by 1 to 2 R_{d}, wherein each carbon atom in C₁₋₄ alkylene is optionally replaced by —N—, —O—, or —S—; R_{d} is selected from the group consisting of halogen, C₁₋₄ alkyl, and —NR_{da}R_{db}; and R_{da} and R_{db} are each independently selected from the group consisting of hydrogen and C₁₋₄ alkyl;
R₄ and R₅ are each independently selected from the group consisting of hydrogen and C₁₋₄ alkyl; and
n is 1, 2, 3 or 4.

3. The compound of claim 1, wherein ring A is selected from the group consisting of phenyl,

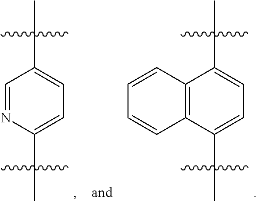 [SMILES: *c1ccc(*)nc1 ; *c1ccc(*)c2ccccc12]

4. The compound of claim 1, wherein R₁ is selected from the group consisting of fluorine, chlorine, bromine, methyl, methoxy, and benzyloxy.

5. The compound of claim 1, wherein R₂ is selected from the group consisting of hydrogen and methyl.

6. The compound of claim 1, wherein R₃ is selected from the group consisting of hydrogen, ethyl,

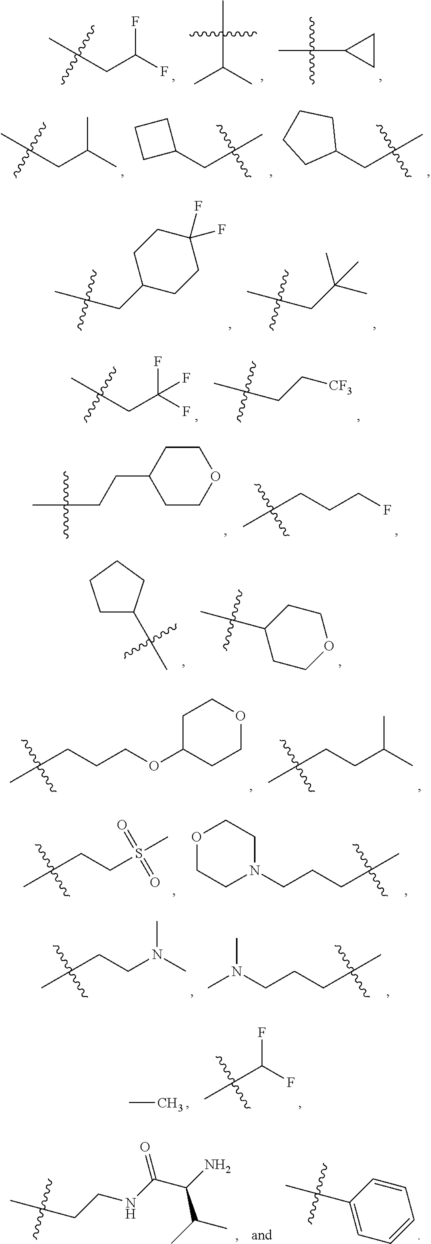

7. The compound of claim 1, wherein R₄ is methyl.

8. The compound of claim 1, wherein R₅ is methyl.

9. The compound of claim 1, wherein R₆ is selected from the group consisting of

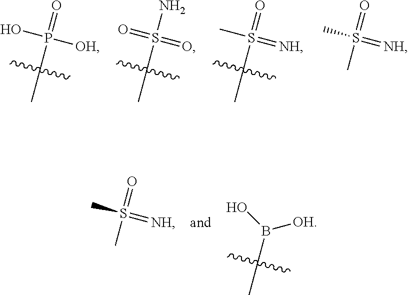

10. The compound of claim 1, having the structure selected from the group consisting of: 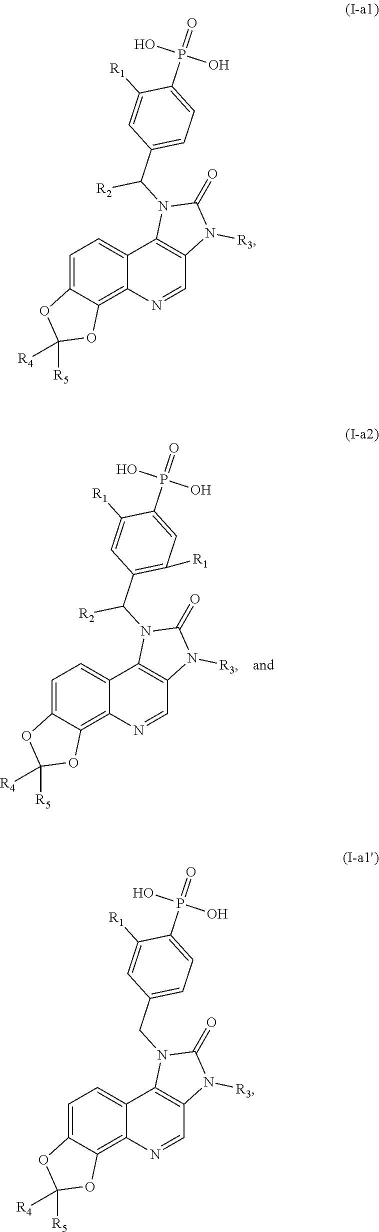
or stereoisomer, a tautomer, or a pharmaceutically acceptable salt thereof, wherein R₁, R₂, R₃, R₄, and R₅ are as defined in claim 1.

11. A compound selected from the group consisting of: 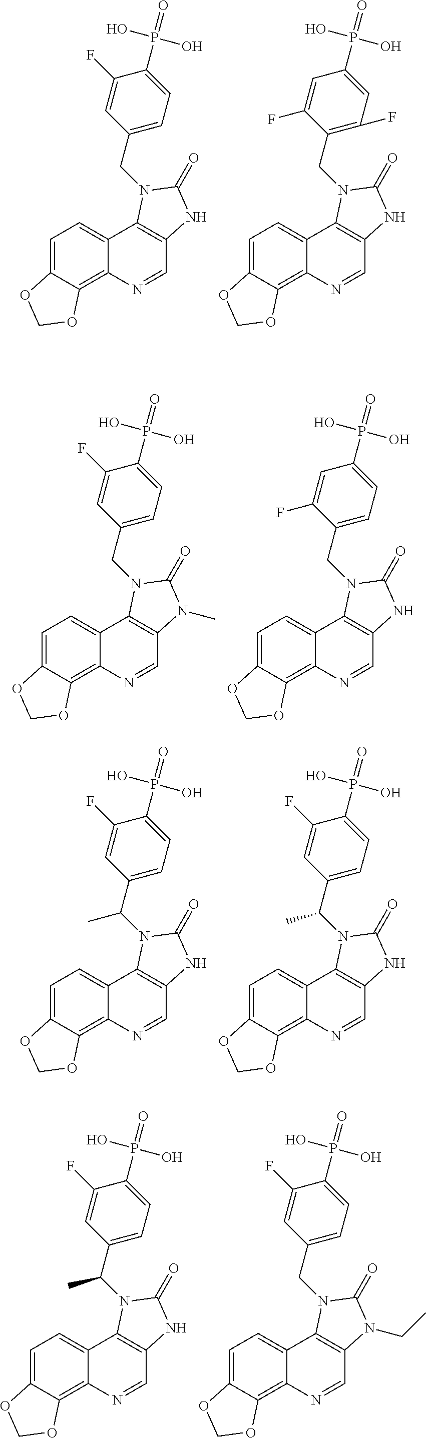 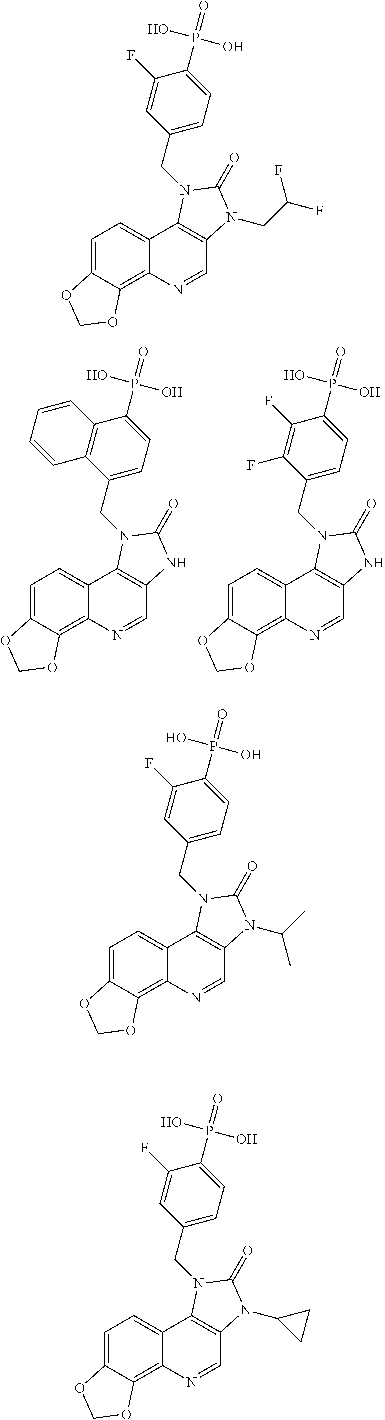 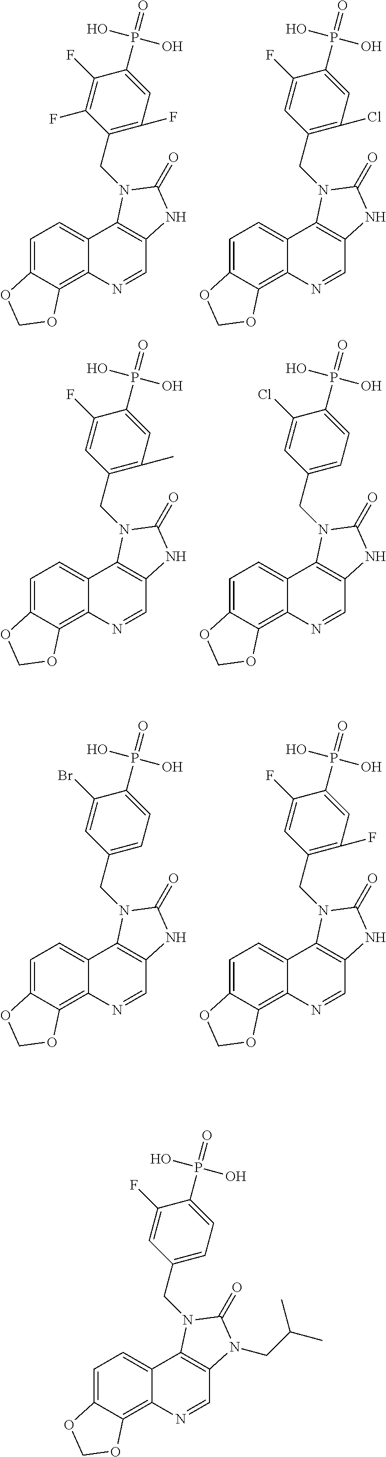 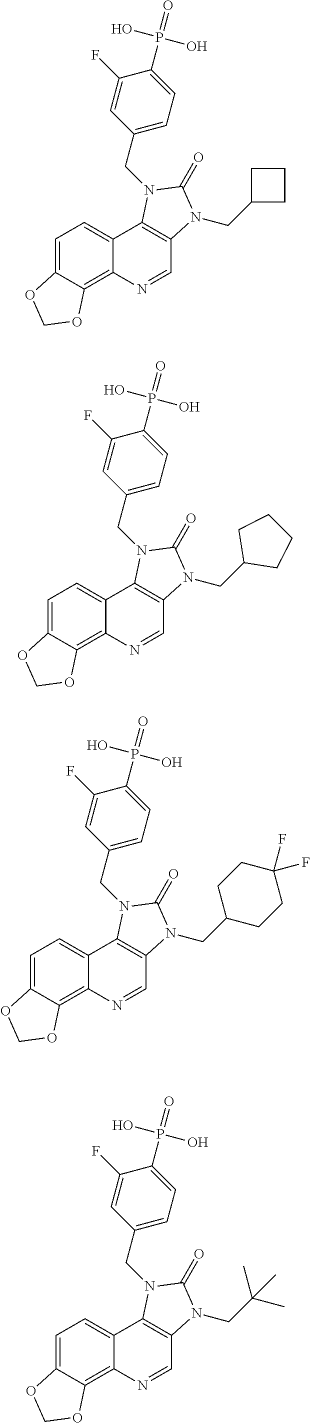 [SMILES: O=c1n(CC2CCC2)c2cnc3c4c(ccc3c2n1Cc1ccc(P(=O)(O)O)c(F)c1)OCO4 ; O=c1n(CC2CCCC2)c2cnc3c4c(ccc3c2n1Cc1ccc(P(=O)(O)O)c(F)c1)OCO4 ; O=c1n(CC2CCC(F)(F)CC2)c2cnc3c4c(ccc3c2n1Cc1ccc(P(=O)(O)O)c(F)c1)OCO4 ; CC(C)(C)Cn1c(=O)n(Cc2ccc(P(=O)(O)O)c(F)c2)c2c3ccc4c(c3ncc21)OCO4] 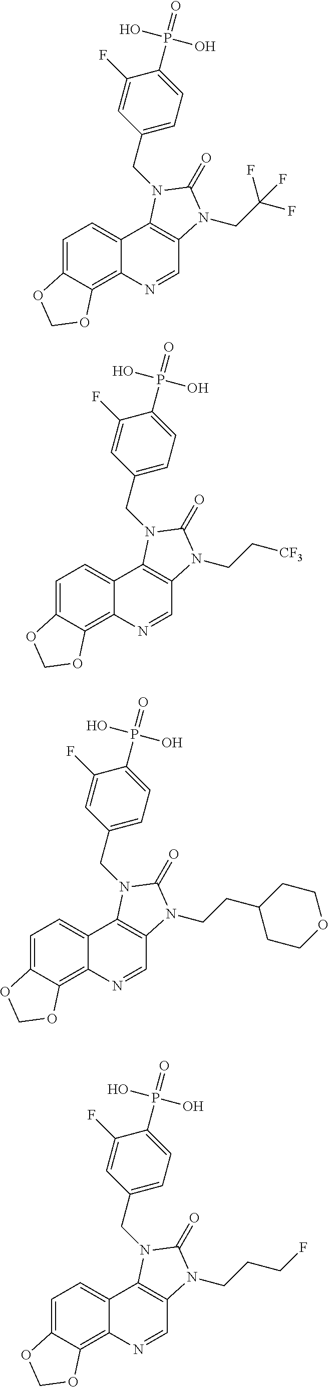 [SMILES: O=c1n(CC(F)(F)F)c2cnc3c4c(ccc3c2n1Cc1ccc(P(=O)(O)O)c(F)c1)OCO4 ; O=c1n(CCC(F)(F)F)c2cnc3c4c(ccc3c2n1Cc1ccc(P(=O)(O)O)c(F)c1)OCO4 ; O=c1n(CCC2CCOCC2)c2cnc3c4c(ccc3c2n1Cc1ccc(P(=O)(O)O)c(F)c1)OCO4 ; O=c1n(CCCF)c2cnc3c4c(ccc3c2n1Cc1ccc(P(=O)(O)O)c(F)c1)OCO4] 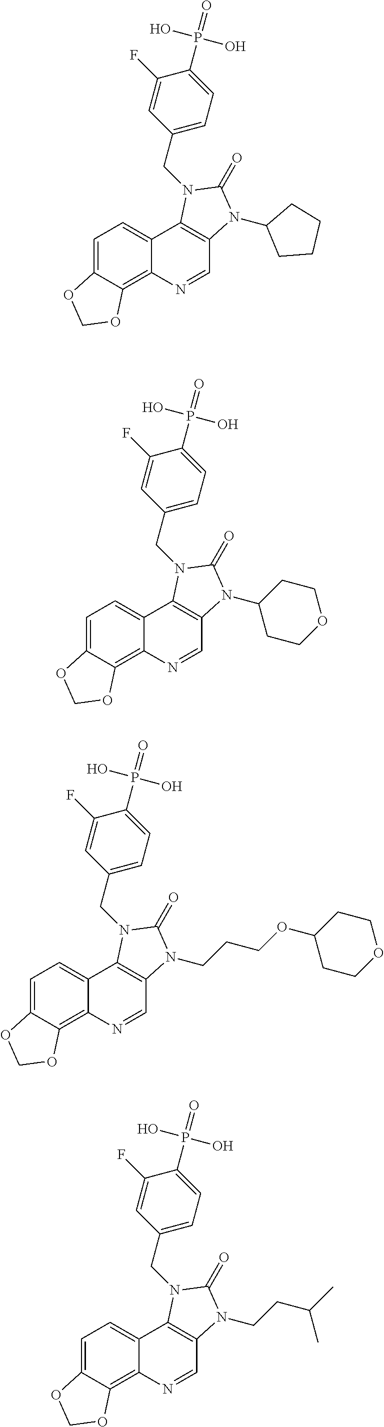 [SMILES: O=c1n(Cc2ccc(P(=O)(O)O)c(F)c2)c2c3ccc4c(c3ncc2n1C1CCCC1)OCO4 ; O=c1n(Cc2ccc(P(=O)(O)O)c(F)c2)c2c3ccc4c(c3ncc2n1C1CCOCC1)OCO4 ; O=c1n(CCCOC2CCOCC2)c2cnc3c4c(ccc3c2n1Cc1ccc(P(=O)(O)O)c(F)c1)OCO4 ; CC(C)CCn1c(=O)n(Cc2ccc(P(=O)(O)O)c(F)c2)c2c3ccc4c(c3ncc21)OCO4] 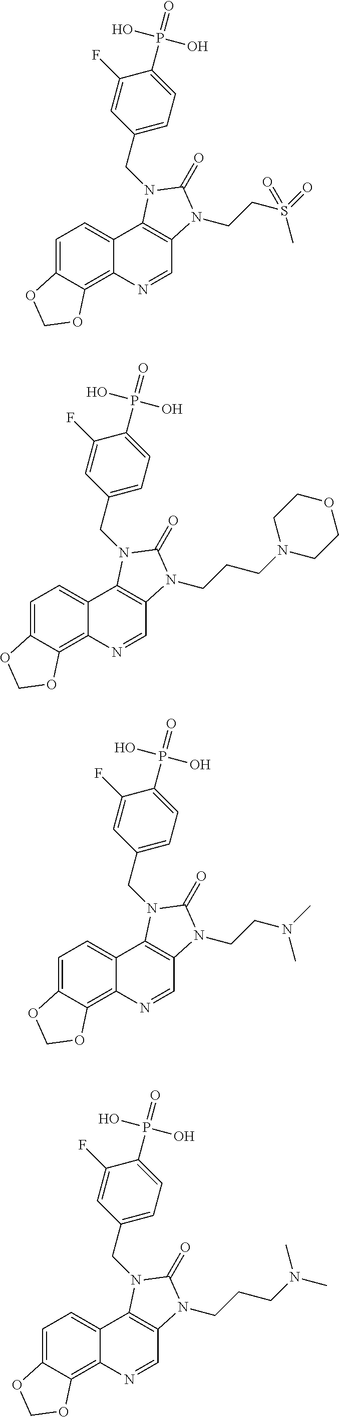 [SMILES: CS(=O)(=O)CCn1c(=O)n(Cc2ccc(P(=O)(O)O)c(F)c2)c2c3ccc4c(c3ncc21)OCO4 ; O=c1n(CCCN2CCOCC2)c2cnc3c4c(ccc3c2n1Cc1ccc(P(=O)(O)O)c(F)c1)OCO4 ; CN(C)CCn1c(=O)n(Cc2ccc(P(=O)(O)O)c(F)c2)c2c3ccc4c(c3ncc21)OCO4 ; CN(C)CCCn1c(=O)n(Cc2ccc(P(=O)(O)O)c(F)c2)c2c3ccc4c(c3ncc21)OCO4] 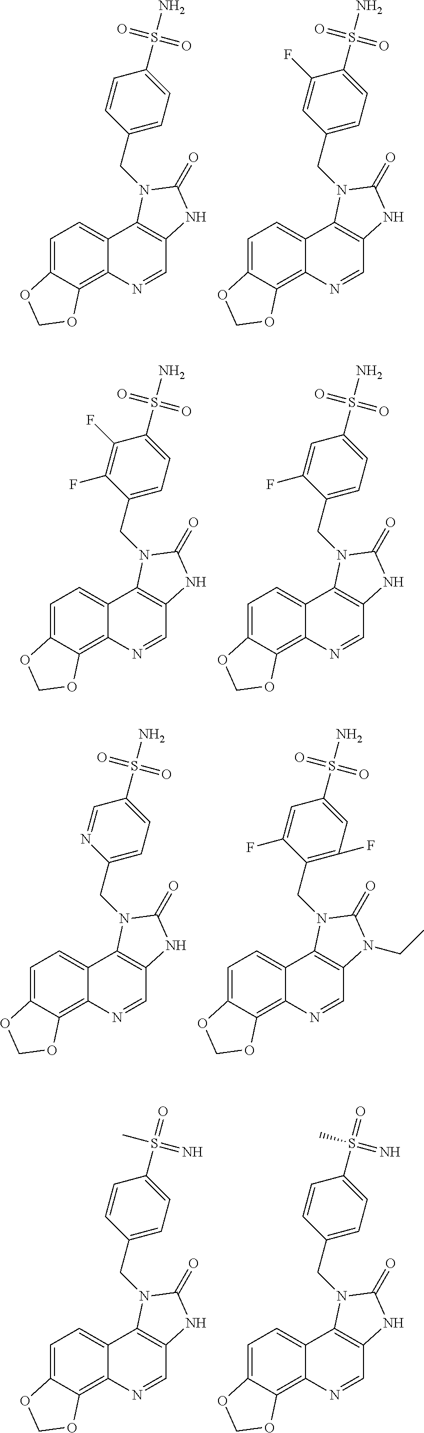 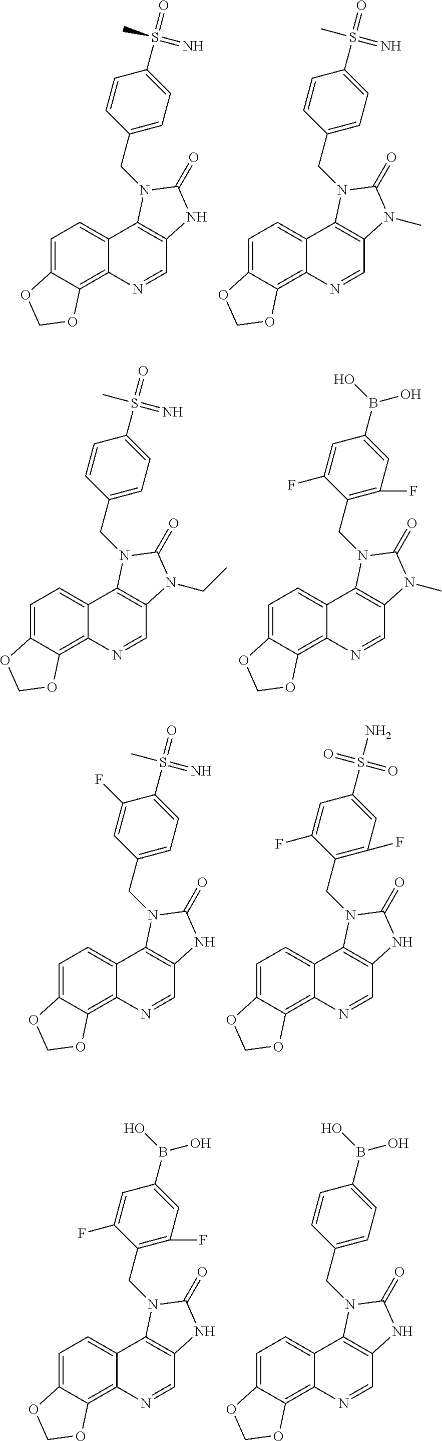 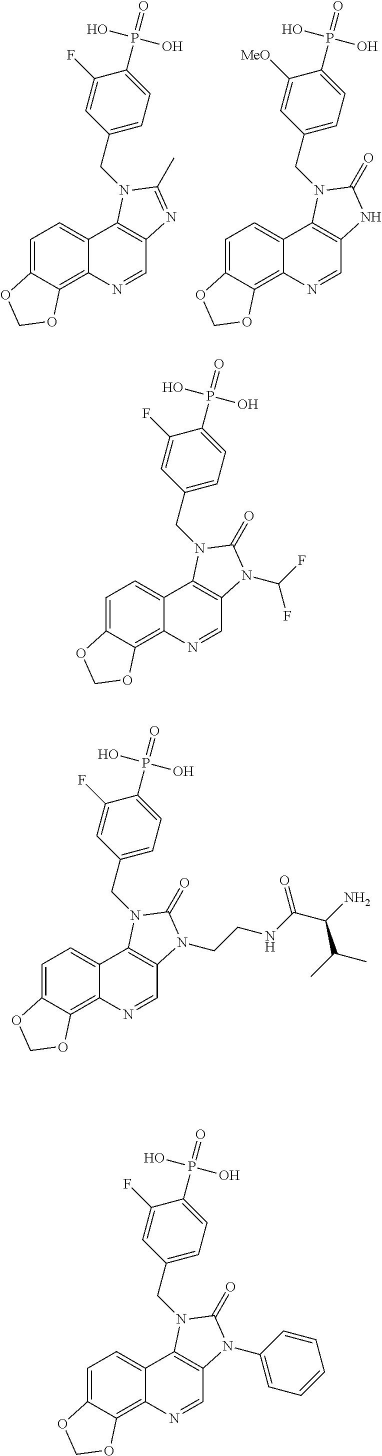 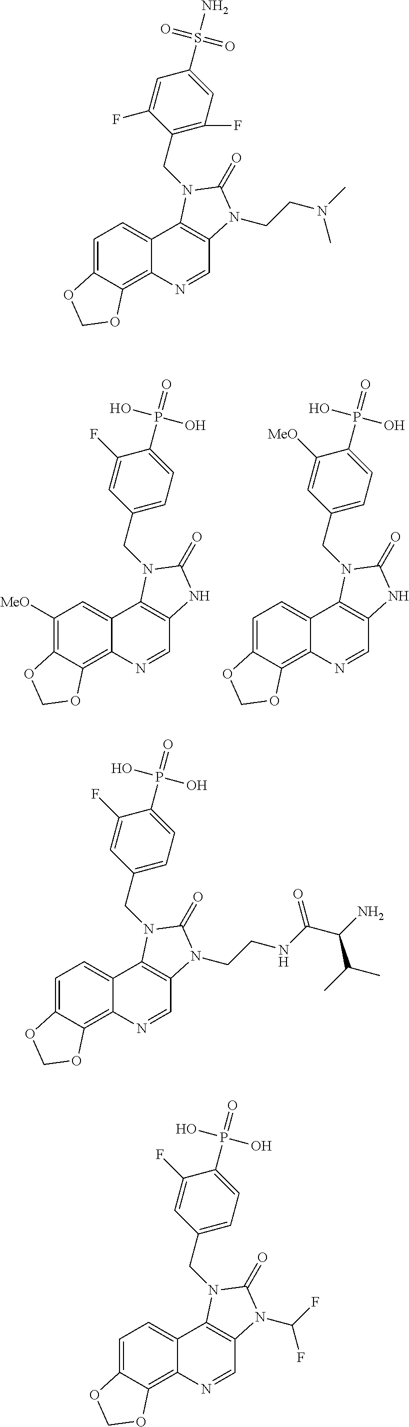 [SMILES: CN(C)CCn1c(=O)n(Cc2c(F)cc(S(N)(=O)=O)cc2F)c2c3ccc4c(c3ncc21)OCO4 ; COc1cc2c(ncc3[nH]c(=O)n(Cc4ccc(P(=O)(O)O)c(F)c4)c32)c2c1OCO2 ; COc1cc(Cn2c(=O)[nH]c3cnc4c5c(ccc4c32)OCO5)ccc1P(=O)(O)O ; CC(C)[C@H](N)C(=O)NCCn1c(=O)n(Cc2ccc(P(=O)(O)O)c(F)c2)c2c3ccc4c(c3ncc21)OCO4 ; O=c1n(Cc2ccc(P(=O)(O)O)c(F)c2)c2c3ccc4c(c3ncc2n1C(F)F)OCO4] 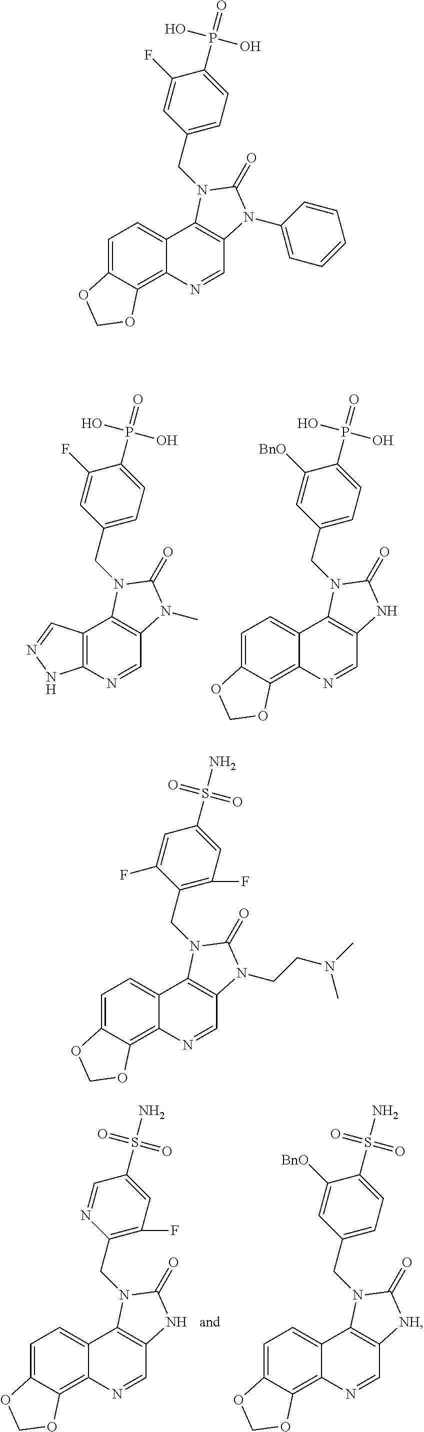
or a stereoisomer, a tautomer, or a pharmaceutically acceptable salt thereof.

12. A pharmaceutical composition, comprising the compound of claim 1, or the stereoisomer, tautomer or pharmaceutically acceptable salt thereof, and a pharmaceutically acceptable carrier.

13. A method for treating an ENPP1-mediated disease, comprising administering the compound of claim 1, or the stereoisomer, tautomer or pharmaceutically acceptable salt thereof, to a subject in need thereof.

14. The method of claim 13, wherein the disease comprises a solid tumor.

15. The compound of claim 1, wherein R₁ is fluorine.

16. The compound of claim 1, wherein R₂ is hydrogen.

17. The compound of claim 1, wherein R₃ is hydrogen.

18. The compound of claim 1, wherein R₆ is

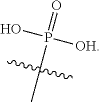 [SMILES: *P(=O)(O)O]

19. A method for treating an ENPP1-mediated disease, comprising administering the pharmaceutical composition of claim 12 to a subject in need thereof.

20. The method of claim 19, wherein the disease comprises a solid tumor.
